(19) United States
(12) Patent Application Publication
(10) Pub. No.: US 2013/0183667 A1
(43) Pub. Date: Jul. 18, 2013

(54) METHODS FOR CANCER SCREENING IN LATIN AMERICAN/HISPANIC POPULATIONS

(71) Applicant: Jeffrey Weitzel, Duarte, CA (US)

(72) Inventor: Jeffrey Weitzel, Duarte, CA (US)

(21) Appl. No.: 13/584,605

(22) Filed: Aug. 13, 2012

Related U.S. Application Data
(60) Provisional application No. 61/522,614, filed on Aug. 11, 2011.

(51) Int. Cl. C12Q 1/68

(52) U.S. Cl. CPC C12Q 1/6886
USPC 435/6.11; 536/24.33

(57) ABSTRACT

Provided herein are novel methods for diagnosing ovarian and breast cancer risk in a Latin American/Hispanic population based on the presence of specific BRCA1 and/or BRCA2 mutations.

### RELATED APPLICATIONS

The present application claims the benefit of U.S. Provisional Patent Application No. 61/522,614 filed Aug. 11, 2011, the disclosure of which is hereby incorporated by reference herein in its entirety, including drawings.

### GOVERNMENT INTEREST

This invention was made with Government support under grant number 1R03CA139588-01 awarded by the National Institutes of Health on March 1, 2009. The Government has certain rights in this invention.

### BACKGROUND

The risk for BRCA mutation carriers to develop breast cancer varies from 57% by age 70 (Chen 2007) to 85% lifetime risk among high risk clinic patients, with lower risks reported from population-based studies (Antoniou 2003). Such carriers have a 20-50% risk for ovarian cancer (Ford 1998). Approximately 13% of all epithelial ovarian cancer cases in Canada have a mutation in the BRCA1 or BRCA2 genes (Zhang 2011). Both BRCA1 and BRCA2 confer susceptibility to ovarian cancer. The cumulative risk of ovarian cancer in women to 70 years of age has been estimated to be 39% in BRCA1 mutation carriers and 11% in BRCA2 mutation carriers (Sunyaev 2001). However, this risk level may vary depending on factors such as the specific mutation, country of residence, and family history (Thompson 2001; Thompson 2002; Antoniou 2003; King 2003).

Genetic testing to evaluate susceptibility to breast and ovarian cancer has recently been gaining acceptance due in part to the increasing number of preventive options for women with BRCA1/BRCA2 mutations and the development of individualized cancer therapies (Narod 2004). The availability of effective screening, treatment, and risk reduction interventions makes BRCA testing a standard of care for patients with a personal and/or family history suggestive of an inherited predisposition to breast and/or ovarian cancer (Weitzel 2003; Domchek 2010; Warner 2011; Weitzel 2011). However, low-income, underinsured, and racial/ethnic minority individuals in the United States and elsewhere have a significant burden of cancer and have limited access to genetic cancer risk assessment (GCRA). For example, although genetic testing is currently offered in many centers in North America, Europe, Australia, and Israel, it is generally not offered in Latin America due to high cost and lack of availability. There is also an overall dearth of Hispanic-specific research, particularly in the area of genetic predisposition to breast cancer. Further, current testing methods generally utilize a DNA sequencing approach wherein heterozygotes (mutation carriers) are identified by DNA sequence analysis. Therefore, there is a need for easier, lower cost genetic screening options for evaluating ovarian breast cancer risk in underserved populations such as Latin American populations.

### SUMMARY

In certain embodiments, methods are provided for detecting susceptibility to ovarian cancer or breast cancer in a Latin American/Hispanic subject by obtaining a nucleic acid sample from the subject and analyzing the sample for one or more mutations selected from the group consisting of BRCA1 3450del4, BRCA1 A1708E, BRCA1 1793delA, BRCA2 S1630X, BRCA2 6252insG, BRCA1 ex9-12del, BRCA1 185delAG, and BRCA1 R1443X, BRCA1 R71G, BRCA1 Q1200X, BRCA1 917delTT, BRCA1 2552delC, BRCA1 S955X, BRCA1 IVS5+1G>A, BRCA1 C1787S, BRCA1 G1788D, BRCA2 3492insT, BRCA2 E49X, BRCA2 9254del5, and BRCA2 G2793R, wherein the presence of one or more of these mutations indicates a susceptibility to ovarian and/or breast cancer. In certain embodiments, the nucleic acid sample is a genomic DNA sample. In certain additional embodiments, kits are provided for carrying out the methods disclosed herein.

### BRIEF DESCRIPTION OF THE DRAWINGS

FIGS. 1-15: Circles indicate women and squares indicate men. Grey circles indicate women affected with ovarian cancer. Black squares indicate men affected with cancer. Diagonal slashes indicate deceased. Ov=ovarian cancer, Br=breast cancer, Gastr=gastric cancer, Pros=prostate cancer, Ut=uterine cancer, Cerv=cervical cancer, PSU=primary site unknown. Numbers prior to abbreviations indicate age at interview, numbers following abbreviations indicate age at diagnosis. Plus sign indicates presence of a germline BRCA1 or BRCA2 mutation.

FIG. 1: Pedigree for subject BOG001.

FIG. 2: Pedigree for subject BOG016.

FIG. 3: Pedigree for subject BOG019.

FIG. 4: Pedigree for subject BOG020.

FIG. 5: Pedigree for subject BOG021.

FIG. 6: Pedigree for subject BOG024.

FIG. 7: Pedigree for subject BOG026.

FIG. 8: Pedigree for subject BOG028.

FIG. 9: Pedigree for subject BOG068.

FIG. 10: Pedigree for subject BOG077.

FIG. 11: Pedigree for subject BOG078.

FIG. 12: Pedigree for subject BOG081.

FIG. 13: Pedigree for subject BOG083.

FIG. 14: Pedigree for subject BOG086.

FIG. 15: Pedigree for subject BOG0101.

### DETAILED DESCRIPTION

The following description of the invention is merely intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein.

Although mutations in the BRCA1 and BRCA2 genes provide a strong indicator of ovarian cancer susceptibility, complete sequencing of both genes is expensive. This expense limits the availability of current genetic screening methods in poor regions (Narod 2009). Testing for founder mutations represents a lower cost alternative for these populations. The presence of founder effects in specific ethnic groups such as Ashkenazi Jewish (Tonin 1996; Warner 1999), Polish (Gorski 2000), and French-Canadian (Tonin 1998) populations has allowed for rapid screening and enhanced mutation detection in these groups.

Hispanics are the fastest growing group in the United States, making up 15.1% of the population. Breast cancer is the most commonly diagnosed cancer in Hispanic women and the leading cause of cancer death. Although the incidence of breast cancer in Hispanics is less than in non-Hispanic whites, previous studies on the prevalence of deleterious mutations in BRCA1 and BRCA2 (BRCA) suggested they may account for a higher proportion of breast cancer in Hispanics than other non-Ashkenazi-Jewish populations (Weitzel 2005; Weitzel 2007). BRCA1 185delAG has been identified previously as a recurrent mutation in Hispanics (Mullineaux 2003; Weitzel 2005; John 2007), occurring on the Jewish haplotype (Makriyianni 2005; Weitzel 2005).

The Hispanic population in the Southwestern U.S. is primarily of Mexican ancestry, whereas individuals of Puerto Rican, Dominican and Cuban ancestry predominate in the Eastern United States. Admixture studies indicate significantly different ancestral populations among U.S. Hispanics (Bertoni 2003; Peralta 2010). It has been suggested that risk for breast cancer is associated with the proportion of European ancestry (Ziv 2006; Fejerman 2008).

The ancestral background of Hispanic women with BRCA mutations has not previously been studied. As disclosed herein, two large cohorts of United States Hispanics were evaluated for BRCA mutations: a clinic-based cohort of patients referred for GCRA, and a population-based cohort selected from a cancer registry population-based study. These studies showed a higher prevalence of BRCA mutations (˜25%) than has previously been reported in Hispanic breast/ovarian cancer families in the United States (Vogel 2007; Hall 2009), as well as a pattern of multiple recurrent mutations in this mostly Mexican-American population. A significant proportion of the observed BRCA mutations were large rearrangement mutations, which are not detectable by standard sequencing (Risch 2006; Walsh 2006; Palma 2008).

BRCA1 ex9-12del, a recurrent large rearrangement mutation initially identified in a small Mexican-American high-risk clinic cohort, was previously unknown (Weitzel 2007). As shown herein, BRCA1 ex9-12del was identified as a Mexican founder mutation with greater Amerindian and less European ancestry than other carriers, accounting for 10-12% of all BRCA1 mutations in the tested Hispanic populations. While most of the recurrent mutations observed in the present studies are likely Spanish in origin, the BRCA1 ex9-12del mutation has never been observed in Spain or South America (de la Hoya 2006; Tones 2009a). All BRCA1 ex9-12del carriers reported Mexican ancestry, and the mutation was estimated to have arisen 1,500 years ago, predating Spanish colonization. Our admixture analyses support this observation showing a higher proportion of Amerindian ancestry and lower proportion of European ancestry among ex9-12del carriers compared to 185delAG carriers. Thus, BRCA1 ex9-12del is clinically significant and one of the most frequent population-specific large rearrangement mutations in the world, as well as the first reported Mexican founder mutation.

BRCA1 185delAG mutations were observed in 9.5% of BRCA carriers. This mutation was first characterized in Ashkenazi Jewish populations, but has since been observed in Middle Eastern and Spanish Jews as well (Atzmon 2010; Velez 2011). The term Hispanos has been applied to the Colonial-Hispanic population (Velez 2011) in the San Luis Valley, encompassing parts of Colorado and New Mexico, and it is suggested that their ancestral origins stem from the immigration of Spanish Conversos and Crypto-Jews (Hordes 2005). While five 185delAG carriers in our study were from New Mexico, and thus likely Hispanos, they reported grandparental ancestry as Mexican or Spanish. Given that the majority of 185delAG carriers in our cohorts were recruited from areas of the US outside of known Colonial-Hispanic settlements, the high prevalence is clinically relevant and may represent a greater than appreciated diaspora of people with Converso and Crypto-Jewish ancestry.

BRCA1 R1443X, which was previously reported as a French-Canadian founder mutation with highly conserved haplotypes (Tonin 1998; Vézina 2005) was observed six times in our study population, with ancestry reported from Mexico (n=4), Colombia (n=1) and Peru (n=1).

Colombia is the second largest country in South America, with a population of nearly 45 million inhabitants. Approximately 1,900 new cases of ovarian cancer are diagnosed in Colombia each year, and the age-standardized incidence of ovarian cancer in Colombia is estimated to be 10.1 cases per 100,000 women per year (versus 11.6 in Canada and 13.2 in whites in the United States) (Globocan 2002, http://www-dep.iarc.fr). A 2007 study of familial breast cancer cases in Colombia identified three founder mutations among 53 families from the Bogota region: two in BRCA1 (3450del4 and A1708E) and one in BRCA2 (3034delACAA) (Torres 2007). In 2009, the frequency of Colombian BRCA founder mutations was estimated to be 4.2% in a sample of 766 unselected breast cancer patients from the Bogota region (Torres 2009b). This study also identified two additional recurrent BRCA2 founder mutations in this population: 6076delGTTA and 6503delTT. However, no previous studies have evaluated BRCA1 and BRCA2 mutations in a Colombian ovarian cancer population.

As disclosed herein, mutation analysis was performed in Colombian subjects with ovarian cancer to determine the prevalence of BRCA founder germline mutations. This analysis utilized a panel of 96 Hispanic mutations that included recurrent mutations reported in the Colombian population. BRCA mutations were identified in 15 out of 96 subjects (15.6%). Specifically, three BRCA1 mutations (3450del4, A1708E, 1793delA) and two BRCA2 mutations (6252insG, S1630X) were identified. The BRCA1 3450del4 founder mutation was observed in 11 of the 15 subjects with BRCA mutations, and accounted for 11.5% of all ovarian cancer cases in the Bogota region. The BRCA1 1793delA mutation has been reported six times in the Breast Cancer Information Core (BIC) database, four times in individuals and Latin American descent and twice in subjects of unspecified ethnicity. The BRCA2 6252insG mutation has been reported twice in the BIC, both times in subjects of Mexican or Spanish descent. The BRCA2 S1630X mutation has been reported six times in the BIC, with two of the cases in subjects of Italian ethnicity and two of English and Western European descent. The present study represents the first time the BRCA1 1793delA and BRCA2 6252insG mutations have been observed in individuals of Colombian descent.

Based on the experimental results disclosed herein, methods are provided for detecting susceptibility to development of ovarian and/or breast cancer in a Latin American/Hispanic subject. In these methods, a nucleic acid sample is obtained from a Latin American/Hispanic subject, and the sample is screened for one or more BRCA1 and/or BRCA2 mutations. In certain embodiments, the sample is screened for one or more mutations selected from the group consisting of BRCA1 3450del4, BRCA1 A1708E, BRCA1 1793delA, BRCA2 S1630X, BRCA2 6252insG, BRCA1 ex9-12del, BRCA1 185delAG, and BRCA1 R1443X, BRCA1 R71G, BRCA1 Q1200X, BRCA1 917delTT, BRCA1 2552delC, BRCA1 S955X, BRCA1 IVS5+1G>A, BRCA1 C1787S, BRCA1 G1788D, BRCA2 3492insT, BRCA2 E49X, BRCA2 9254del5, and BRCA2 G2793R. A subject that exhibits one or more of these mutations is considered to be susceptible to development of ovarian and/or breast cancer.

In certain embodiments of the methods provided herein, the sample is screened for one or more mutations selected from the group consisting of BRCA1 3450del4, BRCA1 A1708E, BRCA1 1793delA, BRCA2 51630X, and BRCA2 6252insG.

In certain embodiments of the methods provided herein, the sample is screened for one or more mutations selected from the group consisting of BRCA1 ex9-12del, BRCA1 185delAG, and BRCA1 R1443X.

In certain embodiments of the methods provided herein, the sample is screened for one or more mutations selected from the group consisting of BRCA1 R71G, BRCA1 Q1200X, BRCA1 917delTT, BRCA1 2552delC, BRCA1 S955X, BRCA1 IVS5+1G>A, BRCA1 C1787S, BRCA1 G1788D, BRCA2 3492insT, BRCA2 E49X, BRCA2 9254del5, and BRCA2 G2793R.

Information obtained using the methods provided herein may be used to formulate or refine diagnosis, treatment, and counseling options for a subject. For example, a subject who is identified as susceptible to the development of ovarian and/or breast cancer may be screened for tumor development to determine whether either cancer is present. Where the subject does not yet display any symptoms of either cancer, they may be targeted for increased monitoring, thereby increasing the likelihood that tumors will be detected early. Susceptible subjects may also be given information regarding self-screening, as well as general information about maintaining a healthy lifestyle and potentially decreasing the risk of or delaying cancer development (e.g., dietary recommendations).

A “Latin American/Hispanic subject” as used herein refers to any subject with a parent or grandparent of Amerindian ancestry. In certain embodiments, a Latin American/Hispanic subject may be Colombian, Mexican, etc. In certain embodiments, a Latin American/Hispanic subject may have a familial predisposition for ovarian or breast cancer, meaning that the subject has a family history of ovarian or breast cancer in a first- or second-degree relative.

“BRCA1 3450del4” as used herein refers to a four nucleotide deletion starting at nucleotide 3450 of the BRCA1 gene of SEQ ID NO:1. or a splice variant thereof.

“BRCA1 A1708E” as used herein refers to a nucleotide substitution in the BRCA1 gene of SEQ ID NO:1 or a splice variant thereof that results in an alanine to glutamine substitution at residue 1708 of the encoded BRCA1 polypeptide (SEQ ID NO:2). For example, BRCA1 A1708E may refer to a cytosine to adenine substitution at nucleotide 64830 of SEQ ID NO:1, alone or in combination with a guanine to adenine substitution at nucleotide 64831.

“BRCA1 1793delA” as used herein refers to a deletion of the adenine at nucleotide 1793 of the BRCA1 gene set forth in SEQ ID NO:1 or in a splice variant thereof.

“BRCA2 S1630X” as used herein refers to a cytosine to guanine substitution at nucleotide 5117 of the BRCA2 gene of SEQ ID NO:3 or a splice variant thereof, wherein this nucleotide substitution results in substitution of the serine at residue 1630 of the encoded BRCA2 polypeptide (SEQ ID NO:4) with a terminator codon.

“BRCA2 6252insG” as used herein refers to an insertion of a guanine between nucleotides 6251 and 6252 of the BRCA2 gene set forth in SEQ ID NO:3 or a splice variant thereof.

“BRCA1 R71G” as used herein refers to a nucleotide substitution in the BRCA1 gene of SEQ ID NO:1 or a splice variant thereof that results in an arginine to glycine substitution at residue 1708 of the encoded BRCA1 polypeptide (SEQ ID NO:2).

“BRCA1 Q1200X” as used herein refers to a nucleotide substitution in the BRCA1 gene of SEQ ID NO:1 or a splice variant thereof that results in substitution of glutamine with any other amino acid at residue 1200 of the encoded BRCA1 polypeptide (SEQ ID NO:2).

“BRCA1 S955X” as used herein refers to a nucleotide substitution in the BRCA1 gene of SEQ ID NO:1 or a splice variant thereof that results in substitution of serine with any other amino acid at residue 955 of the encoded BRCA1 polypeptide (SEQ ID NO:2).

“BRCA1 C1787S” as used herein refers to a nucleotide substitution in the BRCA1 gene of SEQ ID NO:1 or a splice variant thereof that results in a cysteine to serine substitution at residue 1787 of the encoded BRCA1 polypeptide (SEQ ID NO:2).

“BRCA1 G1788D” as used herein refers to a nucleotide substitution in the BRCA1 gene of SEQ ID NO:1 or a splice variant thereof that results in a glycine to aspartic acid substitution at residue 1788 of the encoded BRCA1 polypeptide (SEQ ID NO:2).

“BRCA2 E49X” as used herein refers to a nucleotide substitution in the BRCA2 gene of SEQ ID NO:3 or a splice variant thereof that results in substitution of glutamic acid with any other amino acid at residue 49 of the encoded BRCA2 polypeptide (SEQ ID NO:4).

“BRCA2 G2793R” as used herein refers to a nucleotide substitution in the BRCA2 gene of SEQ ID NO:3 or a splice variant thereof that results in a glycine to arginine substitution at 2793 of the encoded BRCA2 polypeptide (SEQ ID NO:4).

The nucleic acid sample used for BRCA mutation screening can be obtained from any biological sample containing nucleic acid. In certain embodiments, the nucleic sample is obtained from bodily fluid such as blood or saliva. In other embodiments, the nucleic acid sample is obtained from a solid tissue sample, including for example a biopsy sample. In certain embodiments, the nucleic sample is amplified via PCR prior to mutation screening. In certain embodiments, the nucleic acid sample is DNA, including for example genomic DNA or cDNA. In other embodiments, the nucleic acid sample may be RNA, including for example mRNA.

BRCA1 3450del4, BRCA1 A1708E, BRCA1 1793delA, BRCA2 S1630X, BRCA2 6252insG, BRCA1 ex9-12del, BRCA1 185delAG, and BRCA1 R1443X, BRCA1 R71G, BRCA1 Q1200X, BRCA1 917delTT, BRCA1 2552delC, BRCA1 S955X, BRCA1 IVS5+1G>A, BRCA1 C1787S, BRCA1 G1788D, BRCA2 3492insT, BRCA2 E49X, BRCA2 9254del5, and BRCA2 G2793R mutations may be detected using any method known in the art, including for example direct sequencing of all or a portion of the nucleic acid sample, single stranded conformation polymorphism (SSCP) (Hayashi 1991), allele-specific oligonucleotide (ASO) hybridization (Bao 2005), mass spectrometry, allele-specific PCR, denaturing gradient gel electrophoresis (DGGE), RNase protection assay, Southern blot analysis, reverse dot blot, or combinations thereof.

In certain embodiments, screening is carried out using multiplex PCR in combination with mass spectrometry. In these embodiments, a nucleic acid sample is subjected to multiplex PCR using primers that target regions of interest within the BRCA1 or BRCA2 genes, after which remaining nucleotide triphosphates are deactivated using phosphatase. Appropriate multiplex PCR primers may be generated using any method known in the art. For example, primers may be generated using the Sequenom Assay Designer Program. Single base primer extensions are performed, and the single base reaction products are analyzed by mass spectrometry, for example using a Sequenom MassARRAY mass spectrometer. Target mutations are then identified by manual inspection and/or software analysis, for example using Sequenom TyperAnalyzer software. This screening method allows for identification of point mutations, insertions, and deletions.

In certain embodiments, screening is carried out using SSCP. In these embodiments, regions of interest within the BRCA1 or BRCA2 genes are PCR amplified. In certain embodiments, single strands for analysis may be generated by asymmetric PCR. The amplified strands are then analyzed via polyacrylamide gel.

In certain embodiments, kits are provided for diagnosing breast or ovarian cancer risk in a Latin American/Hispanic subject with a familial predisposition for ovarian or breast cancer. In certain of these embodiments, the kit comprises one or more components for screening a nucleic acid sample from a Latin American/Hispanic subject for one or more BRCA1 and/or BRCA2 mutations selected from the group consisting of BRCA1 3450del4, BRCA1 A1708E, BRCA1 1793delA, BRCA2 S1630X, and BRCA2 6252insG, BRCA1 ex9-12del, BRCA1 185delAG, BRCA1 R1443X, BRCA1 R71G, BRCA1 Q1200X, BRCA1 917delTT, BRCA1 2552delC, BRCA1 S955X, BRCA1 IVS5+1G>A, BRCA1 C1787S, BRCA1 G1788D, BRCA2 3492insT, BRCA2 E49X, BRCA2 9254del5, and BRCA2 G2793R. A subject that exhibits one or more of these mutations is considered to be susceptible to development of ovarian and/or breast cancer. In certain embodiments, the one or more components for screening a nucleic acid sample comprise one or more PCR primers designed to amplify a region of the BRCA1 or BRCA2 gene containing one or more of these mutations.

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### EXAMPLES

### Example 1

### Screening for BRCA1 and BRCA2 Mutations in Colombian Ovarian Cancer Subjects

100 unselected Colombian female subjects with ovarian cancer were genetically evaluated in order to identify specific founder germline mutations in the BRCA1 and BRCA2 genes. To ensure that a range of ethnic and social backgrounds were included in the study, ovarian cancer patients were recruited from three public hospitals (Unidad de Cáncer Hospital Departamental de Villavicencio, Oncomédica Montería, Seguro Social Clínica San Pedro Claver) and two private clinics (Hospital Universitario Hernando Moncaleano Perdomo and Instituto de Cancerología de Sucre). Overall, 68% of subjects were recruited from public hospitals and 32% were recruited from private clinics.

Subjects completed an initial risk factor questionnaire during outpatient visits to a medical oncology clinic or during hospital admission, and family histories were recorded with a particular emphasis on ovarian and breast cancer. Five of the subjects (5.2%) had a first or second-degree relative diagnosed with at least one breast cancer and one or more ovarian cancers. The average age of the subjects at the time the questionnaire was completed was 51.8 years (range 16 to 76 years). On average, patients had been diagnosed with ovarian cancer two years prior to completing the initial questionnaire, with a mean diagnosis age of 50 years (range 16 to 75 years). 18.75% of the subjects had been diagnosed at or before age 40, and 55.2% had been diagnosed at or before age 50. Five of the 96 patients had been diagnosed with breast cancer, with four of those having been diagnosed with breast cancer prior to the ovarian cancer diagnosis.

Tumor histology and size and lymph node involvement and grade were abstracted from the medical records. Blood samples were obtained from all subjects, and DNA was extracted from the samples using Puregene DNA extraction kits. DNA quality was only optimal for BRCA testing in 96 of the 100 subjects. BRCA1 and BRCA2 mutations were detected using a novel Hispanic mutation panel predicted to account for up to 90% of Hispanic BRCA mutations (Weitzel 2010). The panel consists of 50 BRCA1 and 46 BRCA2 mutations, including the five recurrent Colombian BRCA mutations reported in the literature (BRCA1 A1708E, BRCA1 3450delCAAG, BRCA2 3034delACAA, BRCA2 6076delGTTA, and BRCA2 6503delTT; Torres 2007; Torres 2009a; Torres 2009b). These mutations are summarized in Table 1.

TABLE 1

| Gene | Mutation | Type |
|---|---|---|
| BRCA1 | 300T/G(C61G) | Point mutation |
| BRCA1 | 5382insC | Insertion |
| BRCA1 | 185delAG | Deletion |
| BRCA1 | 4153delA | Deletion |
| BRCA1 | 943ins10 | Insertion |
| BRCA1 | 3717C/T(Q1200X) | Point mutation |
| BRCA1 | 5242C/A(A1708E) | Point mutation |
| BRCA1 | 4458C/T(Q1447X) | Point mutation |
| BRCA1 | 2552delC | Deletion |
| BRCA1 | 2800delAA | Deletion |
| BRCA1 | 4184del4 | Deletion |
| BRCA1 | 2079A/T(K654X) | Point mutation |
| BRCA1 | 2983C/A(S955X) | Point mutation |
| BRCA1 | 666 + 1(IVS8 + 1G/T) | Point mutation |
| BRCA1 | 2415delAG | Deletion |
| BRCA1 | 3450del4 | Deletion |
| BRCA1 | 1135insA | Insertion |
| BRCA1 | 330A/G(R71G) | Point mutation |
| BRCA1 | 5154del5 | Deletion |
| BRCA1 | 5272-1(IVS18-1G/A/C) | Point mutation |
| BRCA1 | 1205del56 | Deletion |
| BRCA1 | 5420T/A(C1767X) | Point mutation |
| BRCA1 | 5396 + 1G > A (IVS20 + 1G > A) | Point mutation |
| BRCA1 | 917delTT | Deletion |
| BRCA1 | 3148delCT | Deletion |
| BRCA1 | 589delCT(S157X) | Deletion |
| BRCA1 | 2925del4 | Deletion |
| BRCA1 | 5083del19 | Deletion |
| BRCA1 | 4446C/T(R1443X) | Point mutation |
| BRCA1 | 2731C/G_and_T_(P871R) | Point mutation |
| BRCA1 | 2525del4 | Deletion |
| BRCA1 | 1793delA | Deletion |
| BRCA1 | 4671C > T (Q1518X) | Point mutation |
| BRCA1 | 331 + 1(IVS5 + 1G/A) | Point mutation |
| BRCA1 | 3936C > T (Q1273X) | Point mutation |
| BRCA1 | 2576delC | Deletion |
| BRCA1 | 5482G/A(G1788D) | Point mutation |
| BRCA1 | 2156delGinsCC | Deletion |
| BRCA1 | 3878delTA_v3 | Deletion |
| BRCA1 | 5443T > G (M1775R) | Point mutation |
| BRCA1 | 2859del11 | Deletion |
| BRCA1 | 5370C > T (R1751X) | Point mutation |
| BRCA1 | 5105 + 6T > C (IVS16 + 6T > C) | Point mutation |
| BRCA1 | 3596delAAAG | Deletion |
| BRCA1 | 4134G > T (E1339X) | Point mutation |
| BRCA1 | 3124delA | Deletion |
| BRCA1 | 2428C > A (S770X) | Point mutation |
| BRCA1 | 208T > A (L30X) | Point mutation |
| BRCA1 | 2080delA | Deletion |
| BRCA1 | 5472C > T (Q1785X) | Point mutation |
| BRCA1 | 4476 + 1G > A (IVS13 + 1G > A) | Point mutation |
| BRCA1 | 2883delACAG | Deletion |
| BRCA1 | 917delTT | Deletion |
| BRCA1 | 189insTGTC | Insertion |
| BRCA1 | 3587delT | Deletion |
| BRCA1 | 5454delC | Deletion |
| BRCA1 | 3837C > T (Q1240X) | Point mutation |
| BRCA1 | 3118delA | Deletion |
| BRCA2 | 802delAT | Deletion |
| BRCA2 | 4150G/T(E1308X) | Point mutation |
| BRCA2 | 3036del4 | Deletion |
| BRCA2 | 2452C/T(Q742X) | Point mutation |
| BRCA2 | 3710insGA | Insertion |
| BRCA2 | 957del4 | Deletion |
| BRCA2 | 7640del10 | Deletion |
| BRCA2 | 6174delT | Deletion |
| BRCA2 | 6503delTT | Deletion |
| BRCA2 | 7986G/A(W2586X) | Point mutation |
| BRCA2 | 1538del4 | Deletion |
| BRCA2 | 9610C/T(R3128X) | Point mutation |
| BRCA2 | 3492insT | Insertion |
| BRCA2 | 886delGT | Deletion |
| BRCA2 | 5423delT | Deletion |
| BRCA2 | 9254del5 | Deletion |
| BRCA2 | 6027del4 | Deletion |
| BRCA2 | 3417del4 | Deletion |
| BRCA2 | 8550insT | Insertion |
| BRCA2 | 9927del4 | Deletion |
| BRCA2 | 373G/T(E49X) | Point mutation |
| BRCA2 | 999del5 | Deletion |
| BRCA2 | 6006delTG | Deletion |
| BRCA2 | 7261C/T(Q2345X) | Point mutation |
| BRCA2 | 9538delAA | Deletion |
| BRCA2 | 5301insA | Insertion |
| BRCA2 | 5164del4 | Deletion |
| BRCA2 | 2041insA | Insertion |
| BRCA2 | 3767insA | Insertion |
| BRCA2 | 7907delTT | Deletion |
| BRCA2 | 9345G > A (P3039P) | Point mutation |
| BRCA2 | 3394C > T (Q1056X) | Point mutation |
| BRCA2 | 6252insG | Insertion |
| BRCA2 | 7784insC | Insertion |
| BRCA2 | 5844delAGTAA | Deletion |
| BRCA2 | 2663insA | Insertion |
| BRCA2 | 4673del5 | Deletion |
| BRCA2 | 5377delG | Deletion |
| BRCA2 | 3034delAAAC | Deletion |
| BRCA2 | 295 + 2T > C (IVS2 + 2T > C) | Point mutation |
| BRCA2 | 6884C > G (S2219X) | Point mutation |
| BRCA2 | 2604C > A (Y792X) | Point mutation |
| BRCA2 | 9463delG | Deletion |
| BRCA2 | 6633delCTTAA | Deletion |
| BRCA2 | 6076delGTTA | Deletion |
| BRCA2 | 1825delA | Deletion |
| BRCA2 | 4265delCT | Deletion |
| BRCA2 | 6265A > T (K2013X) | Point mutation |
| BRCA2 | 3034delAAAC | Deletion |
| BRCA2 | 7990delATAinsTT | Insertion |
| BRCA2 | 1384delG | Deletion |
| BRCA2 | 4673del5 | Deletion |
| BRCA2 | 4232insA | Insertion |
| BRCA2 | 5117C > G (S1630X) | Point mutation |
| BRCA2 | 4859delA | Deletion |
| BRCA2 | 5025delT | Deletion |
| BRCA2 | 4167C > Pu (Y1313X)C/G/A | Point mutation |

All deleterious mutations were confirmed by direct DNA sequencing. Samples were also genotyped using four multiplex PCR reactions containing Ancestry Informative Markers (AIMs) derived from Kosoy et al. and Saetrom et al. (Kosoy 2009, Saetrom 2009) with Sequenom MassArray technology. Population structure analysis was performed in combination with reference populations that included those of Latin descent to determine ancestry proportions of individuals in the study population.

Overall, 15 of the 96 subjects (15.6%) exhibited a BRCA mutation, with 13 of these exhibiting a mutation in BRCA1 (13.5%) and two exhibiting a mutation in BRCA2 (2.1%) (6252insG). The BRCA1 3450del4 mutation accounted for 11 of the 13 BRCA mutations. The other two BRCA1 mutations were 1793delA and A1708E. The two observed BRCA2 mutations were 6252insG and S1630X. A mutation was observed in two of 18 (11.1%) subjects who had been diagnosed with ovarian cancer at age 40 or below, six of 35 (17.1%) subjects diagnosed at age 41 to 50, and seven of 43 (16.3%) subjects diagnosed after age 50.

A BRCA mutation was observed in seven of eight (87.5%) subjects with a family history of ovarian cancer in a first-degree relative, five of five (100%) subjects with a family history of breast and ovarian cancer in a first- or second-degree relative, four of 20 (20%) subjects with a first- or second-degree relative with gastric cancer, two of seven (28.6%) subjects with a first- or second-degree relative with prostate cancer, and four of 12 (33.3%) subjects with a first- or second-degree relative affected with uterine cancer. Overall, 14 of 15 subjects with BRCA mutation had either at least one first- or second-degree relative affected with breast or ovarian cancer or had at least one first- or second-degree relative affected with gastric, prostate, or uterine cancer. The presence of the BRCA2 6252insG mutation in a family affected with gastric cancer supports the idea that BRCA2 mutations in the ovarian cancer cluster region (OCCR) are associated with a high incidence of gastric cancer in family relatives (Jakubowska 2002). Only one of 33 (3%) of subjects without a family history of cancer was found to carry a BRCA mutation.

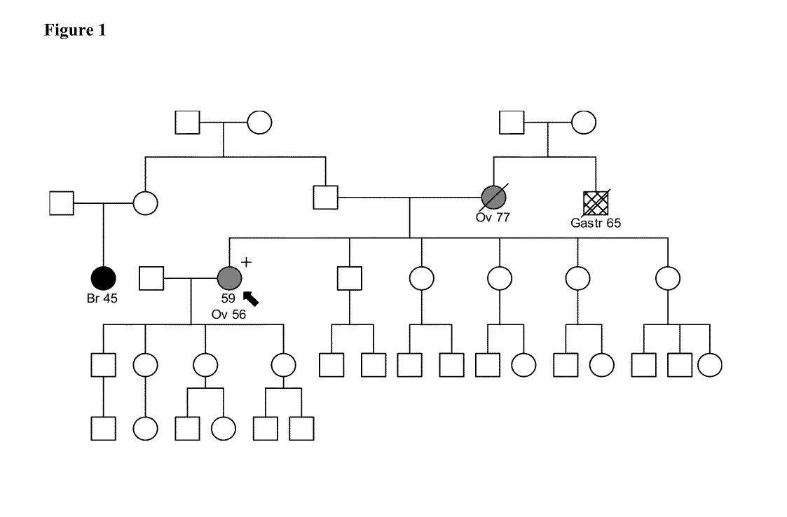

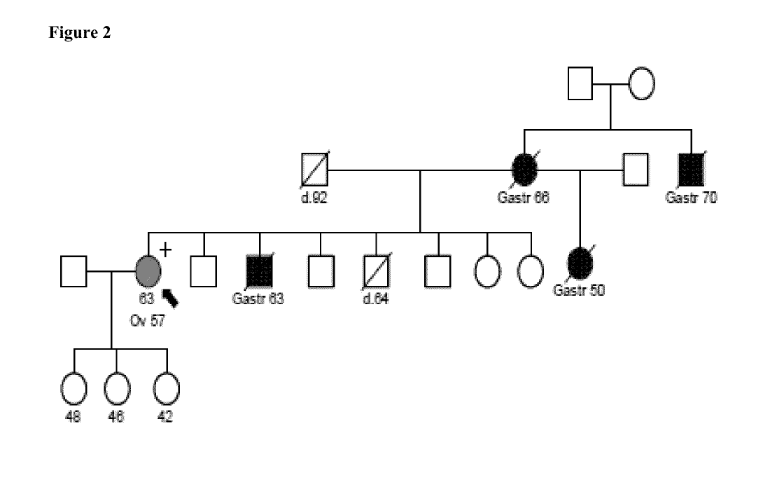

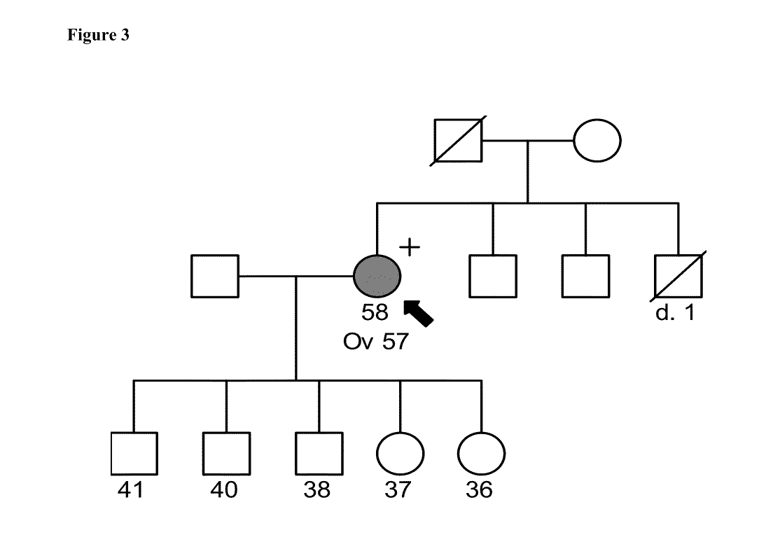

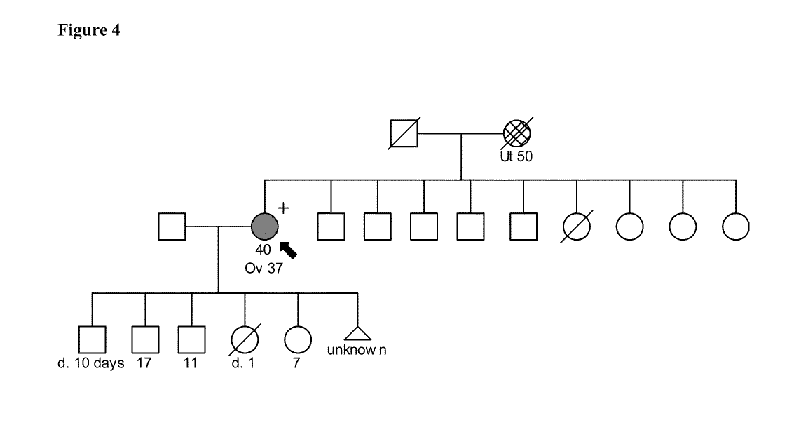

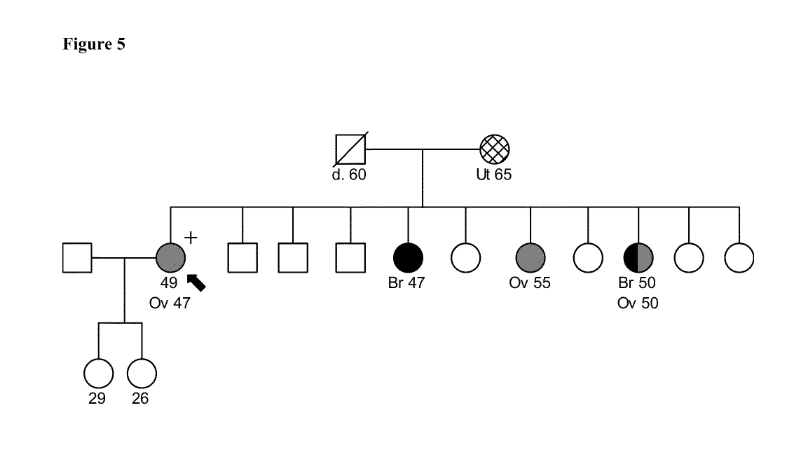

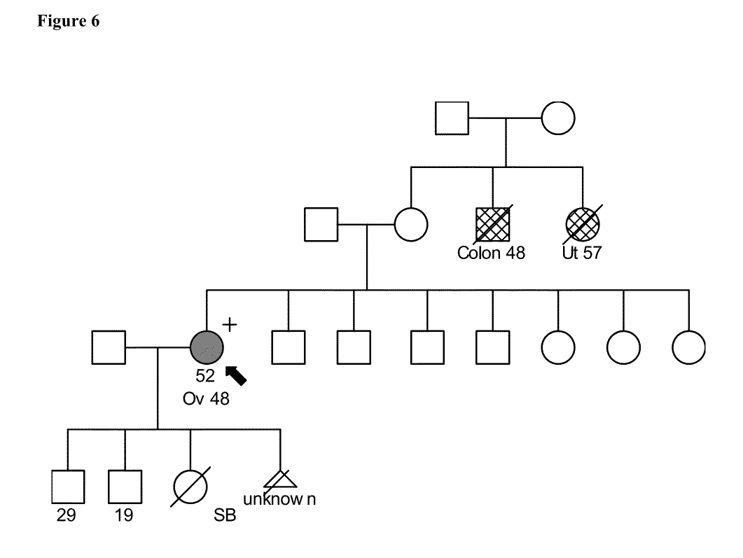

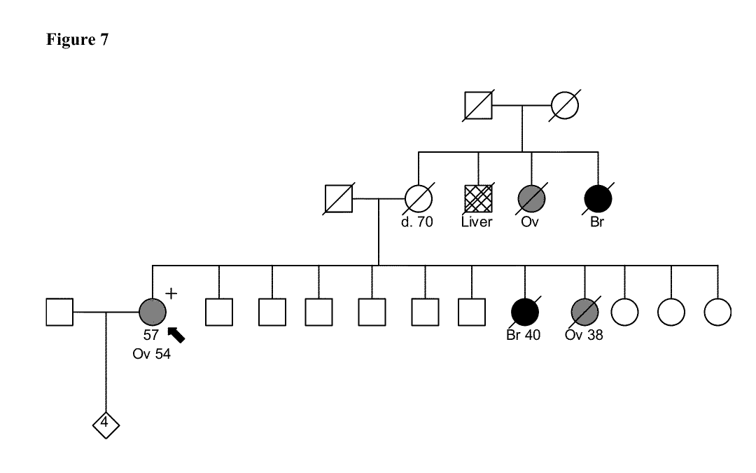

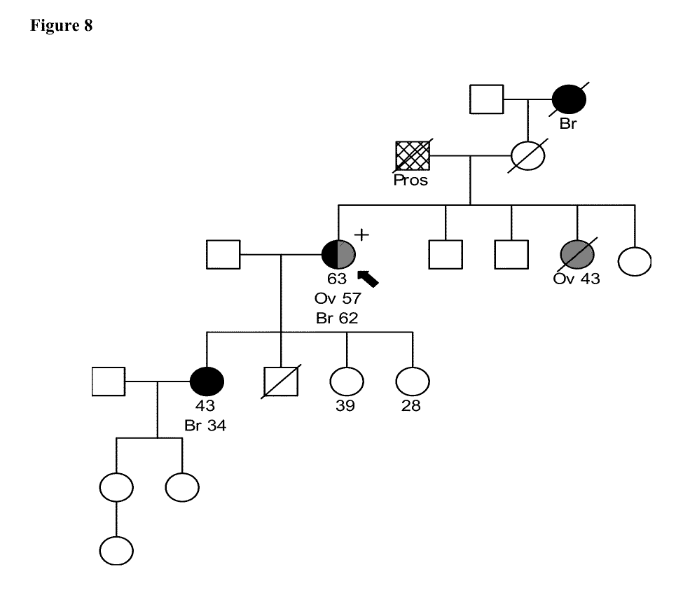

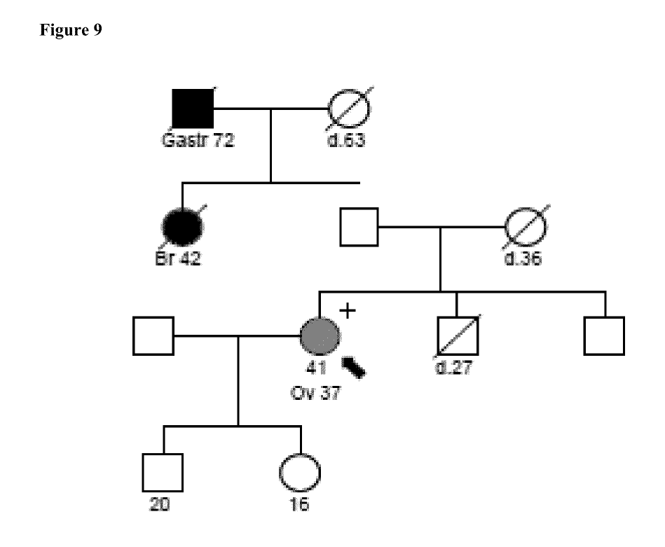

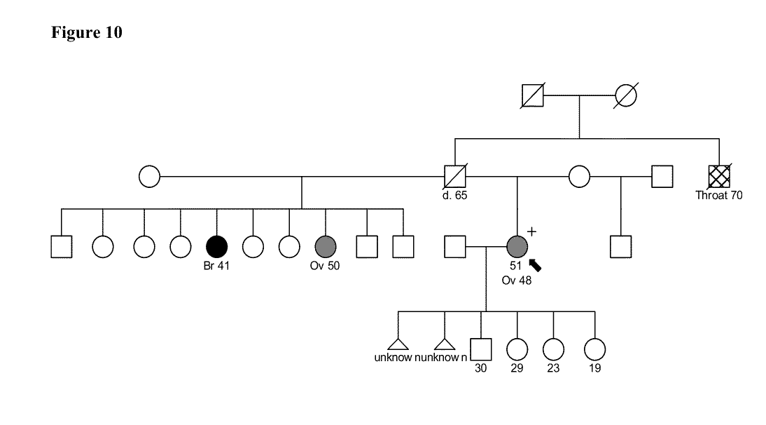

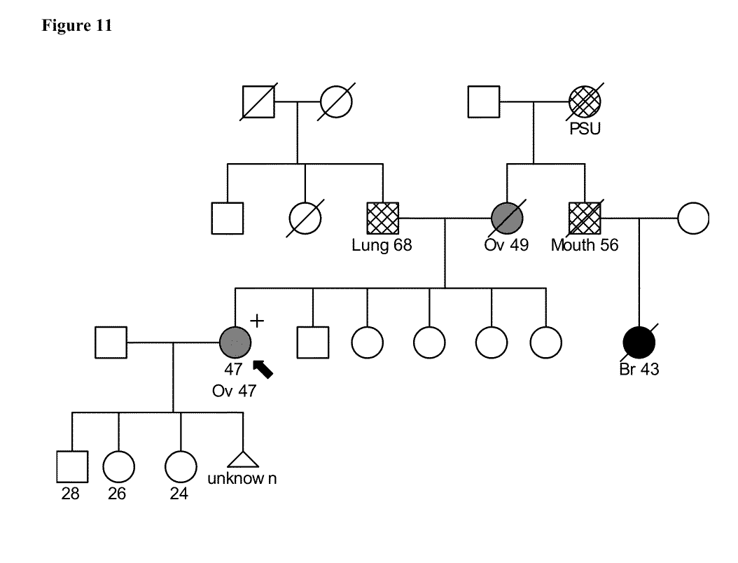

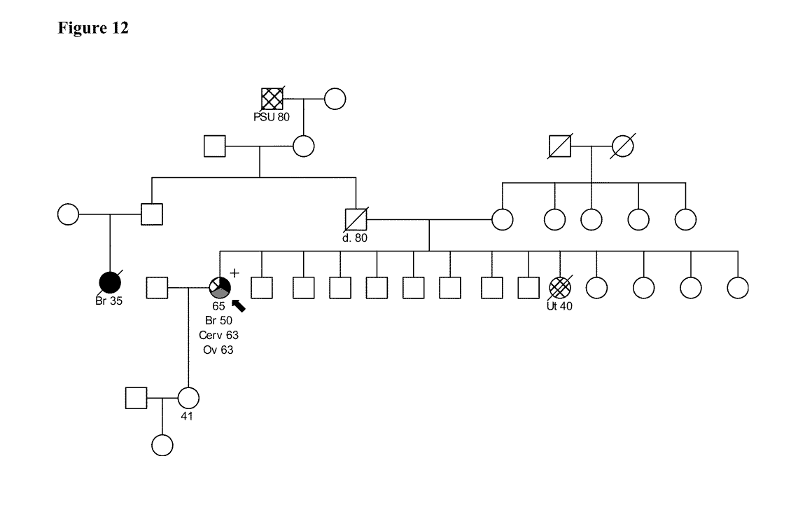

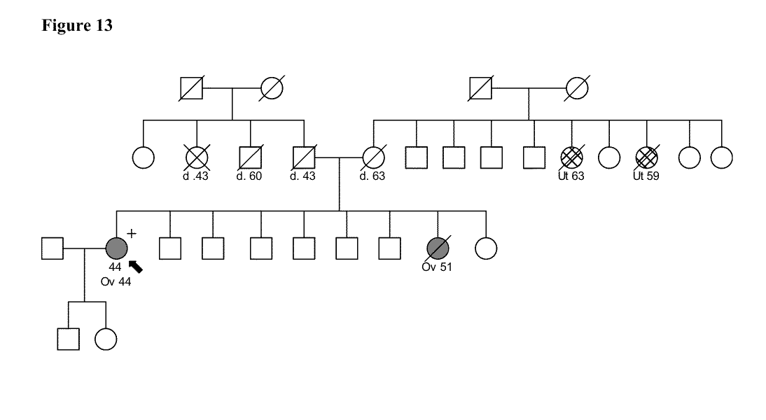

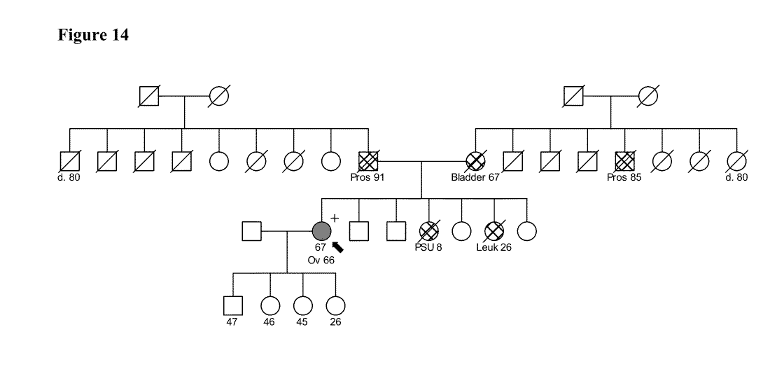

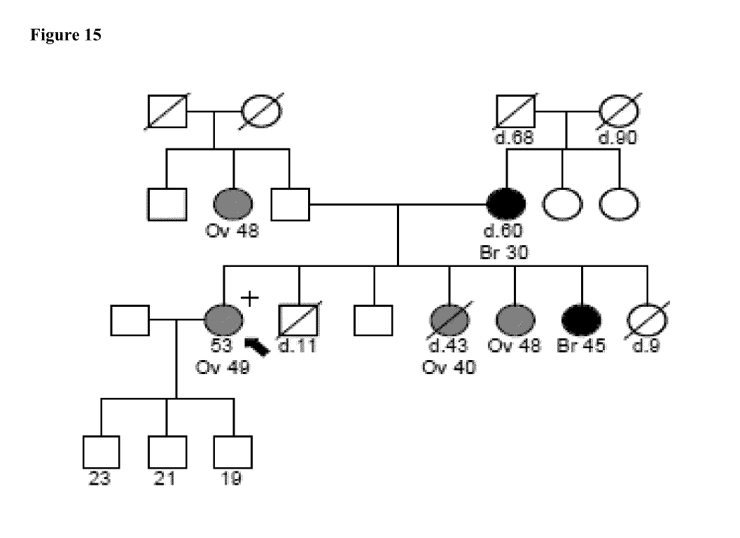

The characteristics of the subjects exhibiting the BRCA mutations are summarized in Table 2. The pedigrees for these subjects are set forth in FIGS. 1-15.

TABLE 2

| | | | | Age at | |
|---|---|---|---|---|---|
| Subject | Gene | Exon | Mutation | diagnosis | Family history |
| BOG001 | BRCA1 | 11 | 3450del4 | 56 | Ovarian cancer, |
| | | | | | gastric cancer |
| BOG016 | BRCA2 | 11 | 6252insG | 57 | Gastric cancer |
| | | | | | (4 cases) |
| BOG019 | BRCA1 | 11 | 3450del4 | 57 | None |
| BOG020 | BRCA1 | 11 | 3450del4 | 37 | Uterine cancer |
| BOG021 | BRCA1 | 11 | 3450del4 | 47 | Breast cancer, |
| | | | | | ovarian cancer |
| | | | | | (2 cases), uterine |
| | | | | | cancer |
| BOG024 | BRCA1 | 11 | 3450del4 | 48 | Gastric cancer, |
| | | | | | uterine cancer |
| BOG026 | BRCA1 | 11 | 3450del4 | 54 | Breast cancer, |
| | | | | | ovarian cancer |
| | | | | | (3 cases), uterine |
| | | | | | cancer |
| BOG028 | BRCA1 | 11 | 3450del4 | 57 | Breast cancer (2 |
| | | | | | cases), ovarian |
| | | | | | cancer, prostate |
| | | | | | cancer |
| BOG068 | BRCA1 | 11 | 1793delA | 37 | Breast cancer, |
| | | | | | gastric cancer |
| BOG077 | BRCA1 | 11 | 3450del4 | 48 | Breast cancer, |
| | | | | | ovarian cancer, |
| | | | | | throat cancer |
| BOG078 | BRCA1 | 11 | 3450del4 | 47 | Ovarian cancer, |
| | | | | | lung cancer, |
| | | | | | mouth cancer |
| BOG081 | BRCA1 | 11 | 3450del4 | 63 | Uterine cancer |
| BOG083 | BRCA1 | 11 | 3450del4 | 44 | Ovarian cancer, |
| | | | | | uterine cancer (2 |
| | | | | | cases) |
| BOG086 | BRCA2 | 11 | S1630X | 66 | Prostate cancer (2 |
| | | | | | cases), bladder |
| | | | | | cancer, leukemia |
| BOG101 | BRCA1 | 18 | A1708E | 49 | Ovarian cancer (3 |
| | | | | | cases), breast |
| | | | | | cancer (2 cases) |

### Example 2

### Clinical-Based Screening for BRCA1 and BRCA2 Mutations in a Hispanic Population

746 female subjects of self-reported Hispanic origin from the City of Hope Clinical Cancer Genetics Community Research Network (CCGCRN) having a personal or family history of breast and/or ovarian cancer were subjected to BRCA testing. The CCGCRN includes a cross-section of cancer center and community-based clinics located primarily in the southwest United States (MacDonald 2010). Testing was offered to subjects who met National Comprehensive Cancer Network (NCCN) criteria (NCCN 2011).

Blood samples, demographic data, and five-generation pedigrees were obtained, including reported ethnicity and country/state of origin for each grandparental lineage. Subjects with mixed ancestry were eligible only if pedigree analysis indicated the Hispanic lineage was the likely origin of the familial cancer pattern. The majority of subjects reported Mexico as their grandparents' country of origin (n=582). Central America (n=80), South America (n=36), the Caribbean (n=13), and Spain (n=35) were also reported. Clinical details were obtained for relatives affected with breast cancer and/or ovarian cancer. Of the 746 subjects, 590 had breast cancer, 39 had ovarian cancer, 20 had both breast cancer and ovarian cancer, and 97 were unaffected (Table 3). The average age at first breast cancer diagnosis was 40 years.

BRCA testing was performed at Myriad Genetic Laboratories, Inc. (Salt Lake City, Utah) and included full sequencing of exons and flanking intronic segments (Frank 2002), analysis of five specific BRCA1 rearrangements, and, for cases that met the vendor's automatic criteria (BRCA mutation probability ≧30%), multiplex quantitative differential PCR (MQDP) (BRCA Analysis Rearrangement Testing [BART], Weitzel 2007) for large rearrangement mutation testing. For cases not meeting the vendor's criteria, BART was conducted electively when covered by private insurance or patient payment. BRCA1 was screened in the remaining uninformative cases by multiplex ligation-dependent probe amplification (MLPA) assay (MRC-Holland, Amsterdam; Sellner 2004). Results are summarized in Table 3.

TABLE 3

| | | Carriers | Non-Carriers | |
|---|---|---|---|---|
| | | Positive | Negative | Variant |
| Total # (%) | | 189 (25%) | 523 (70%) | 34 (5%) |
| Gender | Female | 187 | 520 | 34 |
| | Male | 2 | 3 | 0 |
| Affected subjects | Total # | 169 | 449 | 31 |
| | # with | 144 | 419 | 27 |
| | breast | | | |
| | cancer | | | |
| | # with | 17 | 21 | 1 |
| | ovarian | | | |
| | cancer* | | | |
| | # with | 8 | 9 | 3 |
| | breast and | | | |
| | ovarian | | | |
| | cancer* | | | |
| | Average | 40.0 | 40.8 | 39.5 |
| | age at first | | | |
| | breast | | | |
| | cancer | | | |
| | diagnosis | | | |
| Unaffected | Total # | 20 | 74 | 3 |
| subjects | | | | |
| Country of origin | Mexico | 148 | 412 | 22 |
| | El | 14 | 18 | 3 |
| | Salvador | | | |
| | Guatemala | 8 | 18 | 1 |
| | Spain | 7 | 26 | 2 |
| | Colombia | 3 | 8 | 0 |
| | Peru | 3 | 5 | 1 |
| | Honduras | 2 | 4 | 0 |
| | Argentina | 1 | 6 | 0 |
| | Ecuador | 1 | 2 | 0 |
| | Cuba | 1 | 3 | 0 |
| | Nicaragua | 1 | 3 | 2 |
| | Panama | 0 | 2 | 0 |
| | Costa Rica | 0 | 1 | 1 |
| | Puerto | 0 | 7 | 1 |
| | Rico | | | |
| | Brazil | 0 | 2 | 1 |
| | Belize | 0 | 2 | 0 |
| | Dominican | 0 | 1 | 0 |
| | Republic | | | |
| | Chile | 0 | 3 | 0 |

*Includes Fallopian Tube and Primary Peritoneal Cancer

Deleterious BRCA mutations were detected in 189 of the 746 subjects tested (25%), with 124 subjects exhibiting mutations in BRCA1 and 65 subjects exhibiting mutations in BRCA2. Of these, 21 (11%) exhibited large rearrangement mutations, and 13 of these 21 large rearrangement mutations were BRCA1 ex9-12del. Nine recurrent (seen in ≧4 unrelated families) BRCA mutations accounted for 53% of the total mutations. These mutations are summarized in Table 4.

TABLE 4

| | Breast Cancer | | | | |
|---|---|---|---|---|---|
| | Information | Human Genome | | Grandparental | |
| | Core (BIC) | Variation Society | # of | country of origin | Mexican state |
| Gene | Database Variant | (HGVS) Variant | observations | (# of subjects) | (if known) |
| BRCA1 | 185delAG | c.68_69delAG | 18 | Mexico (16) | Chiapas |
| | | | | Spain (2) | Durango |
| | | | | | Distrito Federal |
| | | | | | Jalisco |
| | | | | | Michoacan |
| | Exon9-12del | c.548- | 13 | Mexico (13) | Chihuahua (2) |
| | | ?_4185 + ?del | | | Durango |
| | | | | | Jalisco |
| | | | | | Puebla |
| | | | | | Tamaulipas |
| | | | | | Veracruz |
| | R71G | c.211A > G | 9 | Mexico (7) | Michoacan (2) |
| | | | | Spain (2) | Sonora |
| | R1443X | c.4327C > T | 6 | Mexico (4) | Oaxaca |
| | | | | Colombia (1) | |
| | | | | Peru (1) | |
| | Q1200X | c.3598C > T | 5 | Mexico (5) | Aguascalientes |
| | | | | | Colima |
| | | | | | Michoacan |
| | 917delTT | c.798_799delTT | 5 | El Salvador (4) | |
| | | | | Guatemala (1) | |
| | 2552delC | c.2433delC | 4 | Mexico (4) | Coahuila |
| | | | | | Guanajuato |
| | S955X | c.2864C > A | 4 | Mexico (4) | Zacatecas (2) |
| | IVS5 + 1G > A | c.212 + 1G > A | 4 | Guatemala | |
| | A1708E | c.5123C > A | 4 | Mexico (3) | Durango |
| | | | | El Salvador (1) | |
| | C1787S & | c.5359T > A & | 4 | Mexico (4) | Chihuahua |
| | G1788D | c.5363G > A | | | |
| BRCA2 | 3492insT | c.3264dupT | 10 | Mexico (10) | Durango |
| | | | | | Guerrero |
| | | | | | Jalisco |
| | | | | | Sinaloa |
| | | | | | Sonora |
| | | | | | Zacatecas (2) |
| | E49X | c.145G > T | 5 | Mexico (5) | Chihuahua |
| | | | | | Nuevo Leon |
| | 9254del5 | c.9026_9030del5 | 5 | El Salvador | |
| | G2793R | c.8377G > A | 4 | Mexico (4) | Durango |
| | | | | | San Luis Potosi |
| | | | | | Zacatecas |

18 subjects had a BRCA1 185delAG mutation (15% of BRCA1 mutation carriers) and 13 subjects had an ex9-12del mutation (10% of BRCA1 carriers). This mutation subset also included R71G (n=9), a Spanish founder mutation (Vega 2001), and R1443X (n=6), a French-Canadian founder mutation (Tonin 1998). The six unrelated Hispanic BRCA1 R1443X mutation carriers shared four distinct haplotypes: two independent haplotypes of Mexican ancestry, one haplotype of Columbian ancestry, and one haplotype of Peruvian ancestry. These were distinct from the haplotype seen in French-Canadian samples (Tonin 1998; Vézina 2005). The BRCA2 3492insT mutation (n=10) accounted for 15% of the BRCA2 mutations. Two recurrent BRCA1 mutations, 917delTT (n=5) and IVS5+1 G>A (n=4), were observed exclusively in subjects with family origins in El Salvador and Guatemala. These mutations were reported previously in Italy (Baudi 2009) and Spain (Llort 2002), respectively. Subjects with the BRCA2 9254del5 mutation (n=5), reported previously in Spain (Campos 2003), were exclusively of El Salvadoran origin.

### Example 3

### Population-Based Screening for BRCA1 Mutations in Subjects of Mexican Ancestry

DNA samples from 492 breast cancer patients of Mexican ancestry were tested for the BRCA1 ex9-l2del large rearrangement. Subjects were identified through the population-based Greater San Francisco Bay Area Cancer Registry and enrolled in the Northern California Breast Cancer Family Registry (NC-BCFR) (John 2004; John 2007). All subjects were negative for sequence-detected BRCA mutations, less than 65 years old, and had a family history of cancer.

To screen for the BRCA1 ex9-l2del large rearrangement, a three primer PCR assay was used (Weitzel 2007). This resulted in co-amplification of the mutant allele 742 by breakpoint fusion product and a 1,145 by wild-type allele product. All BRCA-negative clinic-based cases and NC-BCFR (John 2007) population-based samples were tested.

The BRCA1 ex9-12del mutation was detected in three of the 492 samples, which represents 12% (3/25) of the BRCA1 mutations in the cohort (22 BRCA1 mutations were previously reported in the overall cohort) (John 2007).

### Example 4

### Mutational Age and Admixture Analysis

DNA samples from 18 BRCA1 R1443X carriers and 20 BRCA1 ex9-12del carriers identified in Examples 2 and 3 were genotyped at 12 microsatellite markers spanning 4.1 Mb of chromosome 17q encompassing the BRCA locus. Where possible, haplotypes associated with each mutation were inferred by determining phase from related individuals within each kindred with the same mutation. Primer sequence design and PCR amplifications were performed as previously described (Neuhausen 1996; Neuhausen 1998; Weitzel 2005; Weitzel 2007), with additional microsatellite markers (D17S649, D17S1787, D17S1801, D17S750, D17S951, D17S1860, D17S1861) from the UCSC genome database (http://genome.ucsc.edu/cgi-bin/hgGateway). Mutation age estimation was performed using a previously developed statistical model (Neuhausen 1996).

Among 13 BRCA1 ex9-12del mutation carriers, mutational age analyses estimated the BRCA1 ex9-12del mutation to have arisen 74 generations or 1,480 years ago (95% CI=920-2,260 years).

Ancestry Informative Marker (AIM) SNP genotyping was performed using Sequenom (San Diego, Calif.) MassARRAY genotyping technology and iPLEX chemistry following the manufacturer's protocol (Rioux 2007). Fifty-nine AIMs were developed into two multiplex assays as previously described (Kosoy 2009; Saetrom 2009; Larson 2010). Automated genotype calls were made with MassARRAY® Typer v3.4 software. Genotypes for reference individuals were downloaded from HapMap (http://hapmap.ncbi.nlm.nih.gov/biomart), the Human Genome Diversity Panel (HGDP; http://hagsc.org/hgdp/files.html) H952 dataset (Rosenberg 2006), including 101 African[AFR], 157 European[EUR], 228 East Asian[EAS], and 64 Amerindian[AMI] individuals, as well as HapMap admixed reference populations, Mexican ancestry in Los Angeles (MXL, 49), and African ancestry in the Southwest United States (ASW, 49). In total, 155 BRCA carriers were genotyped with the 59 AIMs. Four SNPs with less than 90% genotype call rate were discarded. Supervised Structure analyses (v2.3.3) (Pritchard 2000) were run with the resulting 55 AIMs on a total of 803 individuals, assigning prior population membership to only HGDP individuals. Runs included 5,000 iterations with 10,000 burn-in cycles under the admixture model with initial a=1.0 and k=4.

Admixture analysis differentiated between four continental populations. The 155 Hispanic BRCA carriers analyzed for AIMs had admixture comparable to the MXL reference population, whereas they were dissimilar to the ASW reference population. Greater Amerindian ancestry was found among the ex9-12del carriers (43%) than among the Mexican 185delAG carriers (30%; p=0.09), and less European ancestry was observed among the ex9-12del carriers (36%) than among the Mexican 185delAG carriers (50%; p=0.03).

The spectrum of mutations in the studied Hispanic cohort was similar in Texas (Vogel 2007), New Mexico, Arizona, and California (John 2007), and the relative proportions of specific recurrent mutations such as BRCA1 185delAG and ex9-12del were the same as those in the population-based series of breast cancer patients enrolled in the NC-BCFR (John 2007), suggesting that the pattern is generalizable and not due to referral bias.

As stated above, the foregoing are merely intended to illustrate the various embodiments of the present invention. As such, the specific modifications discussed above are not to be construed as limitations on the scope of the invention. It will be apparent to one skilled in the art that various equivalents, changes, and modifications may be made without departing from the scope of the invention, and it is understood that such equivalent embodiments are to be included herein. All references cited herein are incorporated by reference as if fully set forth herein.

### REFERENCES

1. Antoniou Am J Hum Genet 72:1117 (2003)
2. Atzmon Am J Hum Genet 86:850 (2010)
3. Bao Nucl Acids Res 33:e15 (2005)
4. Baudi Ann Oncol 20:viii89 (2009)
5. Bertoni Hum Biol 75:1 (2003)
6. Campos Hum Mutat 21:452 (2003)
7. Chen J Clin Oncol 25:1329 (2007)
8. de la Hoya Clin Chem 52:1480 (2006)
9. Domchek JAMA 304:967 (2010)
10. Fejerman Cancer Res 68:9723 (2008)
11. Ford Am J Hum Genet 62:676 (1998)
12. Frank J Clin Oncol 20:1480 (2002)
13. Gorski Am J Hum Genet 66:1963 (2000)
14. Hall Cancer 115:2222 (2009)
15. Hayashi Methods Applic 1:34 (1991)
16. Hordes S M: To the end of the earth: a history of the Crypto-Jews of New Mexico. West Sussex, N.Y., Columbia University Press (2005)
17. John Breast Cancer Res 6:R375 (2004)
18. John JAMA 298:2869 (2007)
19. King Science 302:643 (2003)
20. Kosoy Hum Mutat 30:69 (2009)
21. Larson G, Rivas G, Herzog J, et al: Population admixture analysis of Hispanic women at risk for BRCA1 or BRCA2 mutations in high risk clinic populations, 60th Annual Meeting of ASHG 2010. Washington D.C. (2010)
22. Llort Hum Mutat 19:307 (2002)
23. MacDonald J Natl Compr Canc Netw 8:615 (2010)
24. Makriyianni J Med Genet 42:e27 (2005)
25. Mullineaux Cancer 98:597 (2003)
26. Narod Nat Rev Cancer 4:665 (2004)
27. Narod Salud Publica Mex 51 Suppl 2:s191 (2009)
28. Neuhausen Am J Hum Genet 58:271 (1996)
29. Neuhausen Am J Hum Genet 62:1381 (1998)
30. Palma Cancer Res 68:7006 (2008)
31. Peralta Circ Cardiovasc Genet 3:240 (2010)
32. Pritchard Genetics 155:945 (2000)
33. Rioux Nat Genet 39:596 (2007)
34. Risch J Natl Cancer Inst 98:1694 (2006)
35. Rosenberg Ann Hum Genet 70:841 (2006)
36. Saetrom Cancer Res 69:7459 (2009)
37. Sellner Hum Mutat 23:413 (2004)
38. Sunyaev Hum Mol Genet 10:591 (2001)
39. Thompson Am J Hum Genet 68:410 (2001)
40. Thompson Cancer Epidemiol Biomarkers Prey 11:329 (2002)
41. Tonin Nat Med 2:1179 (1996)
42. Tonin Am J Hum Genet 63:1341 (1998)
43. Torres Breast Cancer Res Treat 103:225 (2007)
44. Torres Breast Cancer Res Treat 117:679 (2009a)
45. Torres Univ Med Bogota (Colombia) 50:297 (2009b)
46. Vega Hum Mutat 17:520 (2001)
47. Velez Hum Genet 131:251 (2011)
48. Vézina Hum Genet 117:119 (2005)
49. Vogel J Clin Oncol 25:4635 (2007)
50. Walsh JAMA 295:1379 (2006)
51. Warner J Natl Cancer Inst 91:1241 (1999)
52. Weitzel: Ancestry-informed strategies for genetic cancer risk assessment, screening and prevention: Adapting the lessons to underserved Latinas, 33rd Annual San Antonio Breast Cancer Symposium. San Antonio, Tex. (2010)
53. Warner J Clin Oncol 29:1664 (2011)
54. Weitzel Arch Surg 138:1323 (2003)
55. Weitzel Cancer Epidemiol Biomarkers Prey 14:1666 (2005)
56. Weitzel Cancer Epidem Biomar 16:1615 (2007)
57. Weitzel Advances in Breast Cancer Risk Assessment, Screening and Prevention: Adapting the Lessons to an Underserved Latina Population, 33rd Annual San Antonio Breast Cancer Symposium; MS3-2 (2010)
58. Weitzel CA-Cancer J Clin 61:327 (2011)
59. Zhang Gynecol Oncol 121:353 (2011)
60. Ziv Cancer Epidem Biomar 15:1878 (2006)

What is claimed is:

1. A method of detecting susceptibility to ovarian cancer or breast cancer in a Latin American/Hispanic subject comprising obtaining a nucleic acid sample from a Latin American/Hispanic subject and analyzing the nucleic acid sample for one or more mutations selected from the group consisting of BRCA1 3450del4, BRCA1 A1708E, BRCA1 1793delA, BRCA2 S1630X, BRCA2 6252insG, BRCA1 ex9-12del, BRCA1 185delAG, BRCA1 R1443X, BRCA1 R71G, BRCA1 Q1200X, BRCA1 917delTT, BRCA1 2552delC, BRCA1 S955X, BRCA1 IVS5+1G>A, BRCA1 C1787S, BRCA1 G1788D, BRCA2 3492insT, BRCA2 E49X, BRCA2 9254del5, and BRCA2 G2793R, wherein the presence of one or more of these mutations indicates a susceptibility to ovarian cancer and/or breast cancer.

2. The method of claim 1, wherein said nucleic acid sample is a genomic DNA sample.

3. The method of claim 1, wherein said one or more mutations are selected from the group consisting of BRCA1 3450del4, BRCA1 A1708E, BRCA1 1793delA, BRCA2 S1630X, and BRCA2 6252insG.

4. The method of claim 1, wherein said one or more mutations are selected from the group consisting of BRCA1 ex9-12del, BRCA1 185delAG, and BRCA1 R1443X.

5. The method of claim 1, wherein said one or more mutations are selected from the group consisting of BRCA1 R71G, BRCA1 Q1200X, BRCA1 917delTT, BRCA1 2552delC, BRCA1 S955X, BRCA1 IVS5+1G>A, BRCA1 C1787S, BRCA1 G1788D, BRCA2 3492insT, BRCA2 E49X, BRCA2 9254del5, and BRCA2 G2793R.

6. A kit for detecting susceptibility to ovarian cancer or breast cancer in a Latin American/Hispanic subject comprising one or more components for screening a nucleic acid sample from a Latin American/Hispanic subject for one or more mutations selected from the group consisting of BRCA1 3450del4, BRCA1 A1708E, BRCA1 1793delA, BRCA2 S1630X, and BRCA2 6252insG, BRCA1 ex9-12del, BRCA1 185delAG, BRCA1 R1443X, BRCA1 R71G, BRCA1 Q1200X, BRCA1 917delTT, BRCA1 2552delC, BRCA1 S955X, BRCA1 IVS5+1G>A, BRCA1 C1787S, BRCA1 G1788D, BRCA2 3492insT, BRCA2 E49X, BRCA2 9254del5, and BRCA2 G2793R.

7. The kit of claim 6, wherein said one or more components for screening a nucleic acid sample comprise one or more PCR primers.


SEQUENCE LISTING (4 sequences)

SEQ ID NO: 1 - DNA, 117143 bp, Homo sapiens
  CDS (4639)..(4718)
  CDS (12955)..(13008)
  CDS (22201)..(22278)
  CDS (23778)..(23866)
  CDS (24473)..(24612)
  CDS (28853)..(28958)
  CDS (31443)..(31488)
  CDS (32810)..(32886)
  CDS (33872)..(37297)
  CDS (37700)..(37788)
  CDS (46156)..(46327)
  CDS (52118)..(52244)
  CDS (54211)..(54401)
  CDS (57494)..(57804)
  CDS (61038)..(61125)
  CDS (64782)..(64859)
  CDS (65360)..(65400)
  CDS (71598)..(71681)
  CDS (77620)..(77674)
  CDS (79543)..(79616)
  CDS (81034)..(81094)
  CDS (82936)..(83057)
  ref <308>: DATABASE ACCESSION NUMBER: L78833.1
  ref <309>: DATABASE ENTRY DATE: 2005-10-12
  ref <313>: RELEVANT RESIDUES IN SEQ ID NO: (1)..(117143)
acggggtctcgaaaaaaggagaatgggatgagaaggatatatgggtagtgtcattttttaacttgcagatttcatcctagtcttccagttatcgtttcctagcactccatgttcccaagatagtgtcaccaccccaaggactctctctcattttctttgcctgggccctctttctactgaggagtcgtggccttccatcagtagaagccggatgttcttgtgtccgaaattggtgggttcttggtctcactgacttcaagaatgaagttgcggaccctcacggtgagtggtacagttcttaaagatgatgtgtccagagtttgttccttctgatgttcggacgtgttcagagttacctccttctggtggattcgtggtctcgctggcttcaggagtgaagctgcagacctttgcggtgagtgttacagctcttaaggcggcatgtctggagtttgttcgttcctcccgtctggagttgttcattcctcctggtgggttcgtggtctcgctggcttcaggagtgaagctgcagacctctgcggtcggtgttaccagcagataaatgctatgcggacccaaagagtgagcagcagcaagatttattgcaaagagcacaagaacaaagcttccacagcgtggaaggagaccagagcgggttgctgctgctggctcaggcagcctgcatttttttttttttttttttttttttttgagatggagtctccctctgtcacccaggctggaatgcagtggtgcaatctgggctcactgcaagctccgcctcccgggttcacgccattctcctgcctcaacctccccagtagaggggattacaggcacccaccaccgcacccagctaatattttgtctttttagtagagtcggggtttcactgtgttagccaggatggtctcgatctcttgacctcgtgatccacccctctaggcctcccaaattgctgggattacaggtgtgagccactggcacccagcggggcagcctgcttttattcccttatctgaccccacccacatcctgttgattggtccattttacagagagctaattggtccgttttgacagggtgctgattggtgcatttacaatccctgagctagatatacacagagtgctgattggtgcatttacaatcctctagctagacataaaaattctccaagtccccactacatttgctagacacagagcactgattggtgcgtttacaaacctttagctagacacagagtgctgattggtgcatttgcaaaccttgagctagacacagagcactgattggtgcatttacaatcctttagctagacacagaagttctccaagtgcccaccagattagctagatacagagtgctgattggtgcatccccaaaccccaagctagacacagagtgctgactggtgcatataaaatcctcaggctagacataaaagttttccaagtccccatctgactcaggagcccagctggcttcacctagtggatcctgcgcagggctgtgccgggcgcctgcactcctctcagcccttgggcagtcgatgggaccgggcgctgaggagcagggggcggtgcccgtcggggaggctcaggccacgctggagctcacaggggttgggagggggctcgggcatggcgggctgcaggtcctgagccttgccctgtgcagggcggctggggcccggtgagaattcaagcggggtgcaggcgggccggcagtgctgggggacccggcgcaccctctgcagctgctggcccgggtgctaggcccctgactgcccggggccgggggtgcggggcccgctgagcccgcgcccacctggaactcgcgctggctggcgagcgctgcgcgcagccccagttcccacacccgcctctccctccacacttccccgcaagcagagggagccggctctggcttcggccagcccagagaggggcccccacagcgcagtggcgggctgaagggctcctccagcacggccagaatggacgccaaggccgaggaggcgccgagagcgagcgagggctgctagcacgttgtcacctcgcattctgaaccacagactctccaactctccggcgcttttcgcccactcggtccctcagaacacgaagggctctctcatcctgtcactaaaacgattagctgtccggagacacggaaaaagtcgcccctcttctttgcaggattcctcccttgaacttctccaaaccctcttagtgtgacgtgaccccacccctagctaacccaggctgcttccttaccagcttcccgccccctggggaggcggcaatgcaaagaccgtccgctgccagctctgccgctatctctgtggggtgaatctaacatggcggacaaagacagtaactagtcccgtttctccgcgttttcgccaagaagattggctcttaccacttgtccctcaaaacgaccaccccattgactggtggcgattgcgtcgacggagacggggcaaaagcaagctgaacccgaaaaataacaaacactggggctgaggggtggaactacgagtgcgcagacatgggccagagcgcatttcccctgccccaggcaaattcggcgctcactgcgtccccgcaggccactgaccttacaagactacttgccccagactcctggggctggatgggaattgtagtctccctaaagagttgtacgtatctttttaaggcctagtttctgctttcaaaatacgaaaacataacactccagtccataactgttgacaagtacaagcgcgcacaggtctccaatctatccactggatttccgtgagaattgtgcccgctctggtattggatgttcctctccataagactacagtttctaaggaacactgtggcgaagacctttcattccgcaacgcatgctggaaataattatttccctccacccccccaacaatccttattacttatatttaccgaaactggagacctccattagggcggaaagagtgggggattgggacctcttcttacgactgctttggacaataggtagcgattctgaccttcgtacagcaattactgtgatgcaataagccgcaactggaagagtagaggctagagggcaggcactttatggcaaactcaggtagaattcttcctcttccgtctctttccttttacgtcatccgggggcagactgggtggccaatccagagccccgagagacgcttggctctttctgtccctcccatcctctgattgtaccttgatttcgtattctgagaggctgctgcttagcggtagccccttggtttccgtggcaacggaaaagcgcgggaattacagataaattaaaactgcgactgcgcggcgtgagctcgctgagacttcctggacgggggacaggctgtggggtttctcagataactgggcccctgcgctcaggaggccttcaccctctgctctgggtaaaggtagtagagtcccgggaaagggacagggggcccaagtgatgctctggggtactggcgtgggagagtggatttccgaagctgacagatgggtattctttgacggggggtaggggcggaacctgagaggcgtaaggcgttgtgaaccctggggaggggggcagtttgtaggtcgcgagggaagcgctgaggatcaggaagggggcactgagtgtccgtgggggaatcctcgtgataggaactggaatatgccttgagggggacactatgtctttaaaaacgtcggctggtcatgaggtcaggagttccagaccagcctgaccaacgtggtgaaactccgtctctactaaaaatacaaaaattagccgggcgtggtgccgctccagctactcaggaggctgaggcaggagaatcgctagaacccgggaggcggaggttgcagtgagccgagatcgcgccattgcactccagcctgggcgacagagcgagactgtctcaaaacaaaacaaaacaaaacaaaacaaaaaacaccggctggtatgtatgagaggatgggaccttgtggaagaagaggtgccaggaatatgtctgggaaggggaggagacaggattttgtgggagggagaacttaagaactggatccatttgcgccattgagaaagcgcaagagggaagtagaggagcgtcagtagtaacagatgctgccggcagggatgtgcttgaggaggatccagagatgagagcaggtcactgggaaaggttaggggcggggaggccttgattggtgttggtttggtcgttgttgattttggttttatgcaagaaaaagaaaacaaccagaaacattggagaaagctaaggctaccaccacctacccggtcagtcactcctctgtagctttctctttcttggagaaaggaaaagacccaaggggttggcagcaatatgtgaaaaaattcagaatttatgttgtctaattacaaaaagcaacttctagaatctttaaaaataaaggacgttgtcattagttctttggtttgtattattctaaaaccttccaaatcttaaatttactttattttaaaatgataaaatgaagttgtcattttataaaccttttaaaaagatatatatatatgtttttctaatgtgttaaagttcattggaacagaaagaaatggatttatctgctcttcgcgttgaagaagtacaaaatgtcattaatgctatgcagaaaatcttagagtgtcccatctggtaagtcagcacaagagtgtattaatttgggattcctatgattatctcctatgcaaatgaacagaattgaccttacatactagggaagaaaagacatgtctagtaagattaggctattgtaattgctgatttccttaactgaagaactttaaaaatatagaaaatgattccttgttctccatccactctgcctctcccactcctctccttttcaacacaaatcctgtggtccgggaaagacagggactctgtcttgattggttctgcactggggcaggaatctagtttagattaactggcattttggcttttcttccagctctaaaacaagctccatcacttgaaatggcaaaataaaatcatggatgaggccgagggcggtggcttatgcctgtaatcccagcactttgggaggccaaggtggtaggatcacgaggtcaggagatcgagaccatcctggccaacatggtgaaaccccctctccactaaaaatacaaaaattagctgggcgtagtggcatgtgcctgtaatcccagctactcaggaggctgaggcaggagaatcacttgaaccaggaggcagatgttgctgtgagccaatatggcaccactgaactccagcgacagagctaaactccatcccaaaaaaaaaaaaaaaaaaaaaaaaacatggatgatcggtgtcgttgagaggataggtatttggaagaacctttgtttgaaactggctctgtacatacaatgaaattacatacttatttacatacaatgaaatgcagaggttttttttttatataggatctctgtcgagaggctggagtgcagtggtgctatcacagctcactgcagcctcaacctcgtcaggctcaagcaatcctcccacctcagcctccagagtagcagggacgataggtgtgcaccaccatgcccagctaatttttgtatttttttttctttttttgagatggagtcttgctctgttgcccaggctggagtgcagtggcgcgatctcagctcactgcaaactctgcctcccgggttcatgccattcttctgcctgagcctcctgaatagctgggactacaagcacccactaccacgcccggctaattttttgtatttttttttcttttttagtagaggcgggatttcaccgtgttagccaggatagtcttgatctcctgaccttgtgatccacccgcctgggcctcccaaagtgctaggattacaggcataagccactgcgtccagccattcttgtatttttctgttgtagagatagggttttgctatgttggccatgctggtctcaaactcctgacctcaagtgatctaccctcccttggcctctcaaggtgctgggattacaggcctgagccattgcacccagccatggtctaaaaatcttgattgaaataccaccttttcatttccagacacccctatttaaaattaccacacccccagcacacactttatcttctattcctgctgcttctccataacactgattactagctgacattctatgtaatgtatccattttttatctctagtcccacagaatgtaaactccaggatgggatttttgttttgtttacatacatctgtatgttcagtagttagaacggtacttgggacctagttgccactcaataaacatttgtcaaataaataataaactaaactaaattagttctttaatttttttaaatatggtgatggttagtagtgagtaacattcaaaaaataagttgaaaagttgtaccattgcctcttacccacaataaaaaagggtaaattcttttctgctttatgaaagttgtttttcatatttgaagtcaagttaatcagattaaggaaaatgtatgttgtgttttcagagcgatacaagatttataaataaccatcctctcccttgcccttcaacattatagctaaacaaaaataagaggaaaacaggattcacaatttatcaatttattgaaaatcagagccagagaagcaggaaatgacattgtaggaaaaaactgcttttgaaaaagcacaaaacttactcatgacaatcagtgatcaggaaaatcctcaatagtgtggcatttggatacatttatgtttcatttccatgggagagagtcataaaaataggatgttctttctcattctggcaaattaaaccatcaattaaaaactcagatacataaaaattaaagatgtaagaatgaaaatgctaaattgttattttcaatcaactattatgttttctagcttttcattgcttttttctgtttcctgttaagattaatttctttttttttttttttttttttttttgagacagactttggctcttgttgcccaggctggagtgcagtggcacaatctcggctcactacaacctccacctcccgggttcaagcaattctgctgcctcagcctccggagtacctgggattgcaggcatgtgccatcacaccagctaattttgtatttttagtagagacagggtttctccatattggtcaggttggtctcgaactcctgacctcaggtgatcctcctgccttggcctccgaaagtgctgggattacaggcgtgagccaccgctcccagactttttgttttgttttgttttgtttttttgagacacggtctcgctctgctgcctaggctggagtgcagtggcacgatcttggctcactgccagctccgactcccgggttcaggccattctcctgcctcagcctcccgagtagctgggactacaggcgcccaccactatgcccggctaattttttgtatttttagtagagacggggtttcaccatgttagccaagatggtctcgatctcctgaccttgtgatccacccgcctcagccttccaaagtgctgggattacagtcctgagccactgcgcccggcctggacctttttttttcggggtggggggttggagtctggctctgtcgcccaggctggagtgcagtggcgccatcttggctcactgcaacctccgcctgccaggttcaagttcaagcgcttctcctgcctcagcctcctgagtagctgggattataggcgcacgccaccgtggccggctaattttgtatttttagtagagatagggtttcatcacgttggtcaggctggtcttgaagtcctgatctcgtgatccacccgcctcggccttccaaagtgctggcgtgagccactgcgcctggcttaagattaatttttgtttgttttgtttttgagacggagtctcgctctttcacccaggccggagtgcagtggcgccatctcggctcactgcaagctccgcctcccgggttcacgccattctcctgcctcggccccccaagtagctgggactacaggcgtccaccaccacgcccggctaattttttgtatttttagtagagacggggtttcaccgtgttagccaggatggtctccacttcctgacctcgtgatccgcccacctcggcctcccaaagtgctgggattacaggcgtgagccaccgcgcccggccttaagattaatttttatggtgttttacattcatttgtatggaaagttctaggatagggatcatatttcacttccttttaatatagtacagtatagcacaatttgcagttatgtcttaatatgtgatcaggaatgatcatgactggaaacagtgttatttgtggtagctatagggtaggtaaggttttcagcctgttttaggtttcttgaactaaaattccttctgctgtcttctaagtcaatattggcagctatttctgacaattggtagttctttgtaactttttacctatgactataacatttttgactttcagaagaatttgctaaaatgtgttccccggtgggttgttgtttttcaacctaaacctagctgctttttccagtcacttatccgtattggaagctcaaaatgcaaatatacagtaggcctaaaatattgcctggtttgaaaagtgtttaaaatatttgaatcatttttatagtaaacatttactctcatcaggacctagaaggggaacattttaattttttttcttttcccttttcacagtcttccttcaacattcattacctttttacatatcggagttttcatctgttcaaagtttgtgtttacagtgtgtttatatagtttagattataattaccatactgaaatataattgtttcagaattgagtcagtggtgagaatgaaagccatctggtatgataactgaatccaatttttcttttacggagaatttctttgaaatgtagcttatctcagaaatagggatttagtaaccaatcagagttttctttgtcaaggttgtttttctttttaaagtcacatttggtcccagtaataataccaatgttggtacaagttatctcaggttgtgaagcatttttcccaagtcatctcaggttgtgaagcattttcccaagtagcatttaattttattcttgcaatagcccaaggagtctggcagggtgaatggcaagagaaggaaacaggttcaggtagagtggttagcccaaggtggctctgcttatatacacaactggtagtagaaacccagcctcctgacttagttcattgtttttcttttcactgccctgtgctatgtcaaaaaccccatgattacaagagttgtattacaacccttcacaataaggttactgtccacaagcttttcttgtgatccttttcttttttttttttctttttttgagatggattctctgtcacccaggctggcccgccttggcctcccaaaatgctgggattacagcgtgagccaccgcacctggcccttgtgatccttttctaaaaagttaaatatttaaggaaaaaaccacattcttgtcacactgccaggttagtcgttctttgatatcttgcctggactttatccaaaaaatccgtttcaaaaattcacatttagagctaagtgtagtggctcacgcctgtaatcccggtcgaggcagatggatcacttgaggtcaggacttcaagaccagcctgggcaatatggtgaaaccccttccctaccaaaaatacaaaaaaattagccgggtgtggcagcacgcgcctgtagtcccagctacttggaatgctgaggcacaagaatcacttcaatccgagaggcagaggttgcagtgagccaagaccacaccactgcactccagcctgagcagcagagtgagtgagactccatctccaaaaaaaaaaaaaaaggttcacattcagaagaaagctaaaggccgggtatagtagctcacacctgtaatcccagcactttgggaagccgaagcaggaagattgcttgatgccaggcattcaagaccagcatgggcatcatagtgagatcctgtctctacaaaaattaattaacattaaaaattaaaaagatggctggcatggtggctcactcctgtaatcccagtactttgggaggccaaggcatggtggtgcatgcctttagtcccagctactcgggaggctgaggcaggagaatcacttgaattcaggaggcggaggttacagagagccgagatggtgccactgcactccagcctgggcgacagaacgagactctgtctgaaaaaaaaaaagaaaattaaaaagaccagaataaagctaaagatttaaaatagcctataggttcctaccagaagttaccagctacctctctgatagtctttccctacaatatcctcctggattattacattttagcaccttgacctatctgatgtcctgcatacacaggcatggtcctgctcagggtttgccttctctgctccctctttcttggaatgctcttcccctaattgttgcatagtgtgtttctttacattattaagctatcctctagtctcacctcagtgaaacctttcctgactccccccatgtacatctcacccccacatagatattgaactacctgtttccccttaccctgcttaatttttctctttaatgcacttattcccatgtattctttaattccgtatcaactgtctaccacactagaatatgagctctatgagagcaggctttattttgtaaactgctacatttctatctcctagaatagtacttgaatatagtagtagatacttaataaacacttgttatattagtataataaatgaactaatctcaggaatgccttggttttgtggatagacaggtagggatgggaacttgggtgatgtattttctgaagtttttatttttaagcttattattattttgagatggagtccagctctgtcgcccaggttggagtacagtggcgcgatcttggctcactgcaacgtgcacttccccggttcaagcgattctcctgccttagcctcccaagtagctgggattacaggcgcatgccaccatgcccagttagttttggtatttttagtagagacagcgtttcactgtgttggccaggctggtctcgaaatcctgacctcatgatccgcccgcctcggcctcccaagtgctgggattacaagcatgagcccccgtgtctggccttattttcttttttttgagacagagtcttcctctgtcacctaggctggagtgcagtggcacgatattggctcactctgcaacctccacctccaggattcaagtgatccttctaccttagtctccaaagtagctgagaccacaggcatgccccaccacgcccggctaatttccgtattttaagcgtagacagggtttcaccatattgtccaggatgatctggaactcctgagctcaggtgatccacccacctcagcctcccaaagtgctaggattacaggcatgaggcaccatgcccggccttaagcttatcattttctaaatttcctttagtgagtacttattacactgtttttacaaagtaatcacaaaccaaacatcatgcctcttctgaagtgatctaataagagtacacagtaccatctgtaaagtgttcttgccagaaagttgaacctgaatgattaagcctgtaagtctagtttataggaaataaggctagaggaacaagttaaacctcaccatagggttatacaatcagcaaaatccagaatgggggaaactccacaggtcaaatgacctaattttaaaaataaatgacaagggagaaaaagtaagagacacctatagatcagaagacacttggggctgggcatggtggctcacacctgtaatcccagcactttgggaggccaaggcaggcggatcacctgaggtcaggagttcaagaccagccggccaacatggtgaaccccaactctactaaaattacgaaaaatcagccgggcgtggtggcgcacgcgtgtagttccaactacctgggaggctgaggcaggagaatcacttgaacttgggaggcagaggttgcagtgagccgagatcgcaccattgcatgccagtctgggctacaaaagcaaaaccccatctcaaaaaaaagaagacacttgggtttgggtgtgttggctcatgcctgtaaaccccgtgctgggaggattgcttgagcccaggagttcaaggctgcagtgaggtatgtttgcaccactgcactccagcctaggtgacagagtgtgaccttatcttaaaagtaataataattaaaataatctggggtaggggtggatatgggtgaaacagcttggccatgagttgatggttgttggaccagggtgatggtccatatagttcattttattattttatttacttgaaattttgaaatacttgaaattttccatattaagttaaaaaggcatttacagtaaacaaaaaaaagttctaggaaggaattcaaaagaaatataagcagaaaattttgtctttatggagcttaaagatgagatgtgcacccacagtgatagtgcagaaaaatatatcactggaaatgaattcgtacgaactattatcaactaatcttttaaatgctgatgatagtatagagtattgaagggatcaatataattctgttttgatatctgaaagctcactgaaggtaaggatcgtattctctgctgtattctcagttcctgacacagcagacatttaataaatattgaacgaacttgaggccttatgttgactcagtcataacagctcaaagttgaacttattcactaagaatagctttatttttaaataaattattgagcctcatttattttctttttctccccccctaccctgctagtctggagttgatcaaggaacctgtctccacaaagtgtgaccacatattttgcaagtaagtttgaatgtgttatgtggctccattattagcttttgtttttgtccttcataacccaggaaacacctaactttatagaagctttactttcttcaattaagtgagaacgaaaaatccaactccatttcattctttctcagagagtatatagttatcaaaagttggttgtaatcatagttcctggtaaagttttgacatatattatctttttttttttttttgagacaaagtctcgctctgtcgcccaggctggagtgcagtggcatgatcttggctcactgcaacctccgccccccgagttcaagcgattcttctacctcagcctcccaggtagctgggactacaggcacccgccaccatgcttggctaatttttgtacttttagtagagataaggtttcaccatattggccaggctggtctcgaactcctgaccttgtgatccacctgcctcggcctcccaaagttctgggattacaggcgtgagccaccacacccgactgacatatattatctattaggatgtaacatcattttgaacagtgttttgtattttttgtgtccatcagtgaaagcaaactgcaagcagttttgaaataagcacattgtgtttgagccttcccagtttctcctttctgttcatttctgcatatccttatgcattcccccttctaagggtcagtgtttgcccgctttgtaatcattgtgaagacaggaaaggacctgataccagtttctatttaggccaaaattcatttatagcagtgattcaagttatatttacgtatttgatgatcttgtcttttgaaatgaaaatgtttgtttcttaataaaagaatttcagaaaaagtagagtaggtaatttagtagaacaagtgggctttctccttttctttatgttaagctatggctcacatcttaccttaaatgtcaactaatttgtttttaagtatttatgtacctggtacataacctggtaccaggtacaaactatgtacttggtaaaaagtttattagcacaaaaaggtatatgatgcaaagtatacttccctcttaccctacaacccctgcctccctgttccctccccagacaaccacaatgatcaatttcttatgtatcctttgaggaatttttaaattccagagttcttaacttggggtttatgaatagtctttatgaatttcctagaattatatttaaattgtattcaaaactatggccatgtacatttttctgggaagatagtccataattttcatctgagtgagctaagatcatgccactgcactccagcctgggagacaagagggagactcaaaaaaaaaaaagaaaggcccagtatttactacagagagctaaagattaacctttaaagccctggggctttcaatttatctggatgagaatctttctggaatgaactgtatgttttatggtcagcttgagtaacaaatgctgagcatactatactattattacagggactcaggggcccagtgtggtagctcctgcccataaccccagcactttgggaggccaaggcaggaggatcacttaaggccaggagttcgaagctgtagtgagctatgatcacaccactgcactccagcctagatgacagagtgagaccctgtctttttttttttttgagatggtgtttcactctattgcccaggctggagtgcagtggtgtgatctcggctcactgcaacctccacctcctgggttcaagcgattctcctgcctcagcctcttgagtagctgggattacaggcatctgccaccacacccagctaatttttgtatttttagtcgagacagggttttcaccatgttggccaggctgctctcaaactcctgacttcagctaccttggccttaaaaagtgttgggattacaggtgtaagccaccgcgcctggctgaccctgtctcttaacaaaaaaagagagattaagttatgaatatagttgctttgagaacttgtggaagaaggaaattataggcttataggcagagataataatacgagcaaatgtacaaataaaagaaaatagaggacgggcgcggtggctcacgcctataataccagcactttgggaggtcgaggtgggcggatcacgaggtcaggaaattaagaccatcctggccaaaatggcgaaacactgtctctactaaaacacacaaaaaactagcctggcatggtggcacgtacctgtagtcccagctacttggtaggctgcggcaggggtatcacttgaacctgggaggcagaggttgccctgagccgagatcatgccaatgcactccagcctgacaacagagtgagactctgtctgaaaaaaaagaaaagaaaagaaaatacatccaggaaaaataagctaactttgcatatgtgtataggagttgtgttagaaaaggaagaagccctcaaagatgggaagccatttgcaagaaagagaaggtccaagaggaggcagaagggattggaaatagaaaaaggatgtaagaaagagttgattattactcataaacagtaatgaaggaaaaggagagtaattctacaggaagatgctgaggtgctttgagcccagtgaagttggaggtaaagacagctgttgaggccgggcacggtggctcacgcctctaatcctagcacttttggagcccaaggcaggtggatcacctgaggtcaggagctcaagaccagcctgaccaacatagagaaaccccatctctactaaaaatacaaaattagacgggcgtggaggcgcatgcctgtaatcccagctacttgggaggctgaggcaggagaatcacttgaacctgggaggcggaggttgcagtgagccgagattgcgccattgcactccagcctggccgacaagagtgaaaactgtctcaaaaaaaaaaaacaacaaaaaacagctgttgagattgagaggattagagttggcaactggagaagagtgagaagcttggtttcaagcttgtgatagtcaggattgtgatagtcaggaaagaaccagtcataaagatatatgtgtgtgtatacatataaatatgttatatatatgtgtgtgtgtgacacatatatatttttgtttgtttctttgagacagtgtctccctctgacacccaggctggagttcagtggtgtgatcatagttcacttttaccttgcaatctgggttcaagcaatctctcatctcagcccctcaagtagctaggactacaggtacatggcatttgcccagctaatttttaagtttcttgtagagatgggccagccatattttaaattgtgttttgaatgttatattagaattaaaagtccaaagccgggtgtggtggctcacgcctgtaatcccagcactttgggaggctgaggtgggcggatcacgaggtcaggagttcgagaccagcctggccaatatggtaacaccatctctactaaaaatacaaaaattagctgggtatgggggcacatgcctgtagtcccagctactcaggaggctgaggcagaggaacctcttgaacccaggaggcagaggctgcagtgagttgagatcgtgccactgtactctagcctgggcgacagagcaagattccgtctcaaaaaaaaaaaaagtccagtataatgcccatgtgatagatcgactttttcatgaaatctcttctgtaatatcaatataatctgaataacactttgatctatatgatgagaaagctgggagcctgggagcgatacccccatgcttttgttgtattaattgtattttctacggataaactctaattgctaaaaataaaacaactttattgacccaagcaagcctaaagttctgaaatcttttttttatttttgtttgtttgtttgtttgtttttgtttgttttgttttgagacggagtctcgctctgtcgcccaggctggagtgcggtggtgcagtctcggctcactgcaagctccacctcccgggttcacaccattctcctgcctcagcctcccaagtagctgggactacagacgcctgccaccacgcccagctaatttttttgtatttttagtagagaaagggtttcaccgtgttagccaggatggtctcgatctcctgacctcgtgatctgcccgccttggcctccctaagttctgggattacaagtgtgagccaccacgcccggctgtttttttttgttttgttttgagacggagtctcactgtgttgcccagactggagtgcagtggcatgatctcagctcactgccacctccatctcctgggttcaagcaaatctcctgcctcagcctcccgagtagctgggactacaggcatgtgccaccacacctggctaatttttgtatttttagtagagacggggtttcactatgttggccaggctggtccaaaactcctgacctcaggtgatctgctcgccttggcctcccacagtgccaggattacaggcatgagccaccttgcccagccagttctgaaatcttttatgaagcctataaaaaaagataataataccaatctagaaaatatttcttaaggcagtcatgcattagtttgaactttccaaacaaaaaaatgcaatgtgtaatacttttttttttttttttgagatggagtcttgttctgttgcccaggctggagtgcagtggtacaatctcggctcactgcagcctctgcctctctggttcaagtgattctcctgcctcagcctcccaagtagctgggattacaggcgtgcaccaccatgcatggctaatttttgtatttttagtagagacagggtttcaccatgttgacaaggctgatctcgaactcctgacctcaggtgatccgcccacctcagcctcccaaagtgctgagattacaggcattagccaccacgcccagccttttattttagtagagaccatgtttcaccatgttgaccaagctggtcttgagctgacctcaagtgatccgcccacctccacctcccaaaatggtgggattataggcatgagccaccgcacccagcctgtaatacttttttgaagatctagaaccacattgttcaaagagatagaatgtgagcaataaatgtaacttaaatttttcaacagctactttttttttttttttttgagacagggtcttactctgttgtcccagctggagtacagtggtgcgatcatgaggcttactgttgccttgacctcctaggctcaagcgatcctatcacctcagtctcccaagtagctgggactgtaagtgcacaccaccatatccagctaaattttgtgttttctgtagagacggggtttcgccatgtttcccaggctggtcttgaactttgggcttaacccgtctgcccacctaggcatcccaaagtgctaggattacaggtgtgagtcatcatgcctggccagtattttagttagctctgtcttttcaagtcatatacaagttcattttcttttaagtttagttaacaacctttatacatgtattctttttctagcataaagaaagattcgaggccgggtgcggtggctcacgcctgtaatcccagcactttgggaggctgagatgggcacatcacgaggtcaggagatcgagaccatcctggctaacatggtgaaaccccgcctctactaaaattacaaaaagttagccaggcgtggtagcgggcacctgtagtcccagctactcaggaggctgaggcaggagaatggcgtgaacccaggaggcagagcttgcagtgagcagagattgtgccactgcactccagcctgagagacagagcgagactccgtctaaaaaaaaaaaaaaagattcgaatccttatcttggttgatttttgcgtatctagttccactgaattatttatataattgtatagactacagcacgagacagcttagcttgtcactctactgtactatattctgcagtactatcataagggaatttcctccctacccctgctctgaattgttcaattgtactatttgctggagtaatgcttgatgccttcttgatccattatactagagtatatgtagtatttgtagattctgaaggagtgggagcctctattctgagttttaaaggtacttatgtacagtggaggtagctttttgacagcctcatcttccaaactatagagtcattgttttgttgagtgcaatatggtacttgaagcatctatatcggcgaagaaggacccaagtctccttgaccttacctacctacattcactttctctggtaggaagattgtgggtgcctctctccagacttagtttccatgtcaaaaaagaaaaaaggaagattgtgggctttgctacaatccaattctggatccaatataaccttcattgcttaattactgtgtgatctgggacaagcctctactctataaaaatgaagataaggccaggcttgatggctcatgcctgtaatcccagcacgttgggatgccaaggcaggaggatcacttgaggtcaggagttcgagaccagactgggcaatatagtgaaaccacatctgtacaaaaataaagatagaaagtagcccagcgcaatggctcacacctgtaatcccagcactttgggaggctgaagcaggcgatcacttgaggtcgggagttcaagactgtagacagatagataggtaggtagatagatagagatatagatatagttggggtttttttgttttgttttgttttgtttttgagatggagtttcgctcttgttgcccaggctggagtgcaatggcgcgatctcagtttactgcaacctccgcctcccgggttcaagagattctcctgcctcagcctcctgagtagccaggattacaggcatatgccaccatgcccggctaatttttgtatttttagtagagacagggtttctccgtgttggtcaggctggtcttgaactcctgacctctcccaaagtgttgggattacaggcgtgagccaccgctcctggccttttttttttttttttttttttttttgagacagagtcttcctctgttgcccagggtggagtgcagtggcactcttctcagctcattgcaacctctgccatcctgggttccagtgattctcatgcctcagcctcccaagtagctgggactcaggcgtgtgcccaccacgcctggctaattttgttgtatttttagtagagacagggtttcaccatgttagccaggctggtctcaaactccaggcctcaagtgatctgcctgcctcagcctcctgggattgcagacatgagccactgcacccggccaagagagggtaataaatgttaaattacctggctagtaaaaaatattctctaagtgtcttttctcacaattcccaatgccttttttttttttttggcacaatctcactctgttgcccaggctggaatgcaatggtgcaatattggctcactgtaacccccgcctcacaggttcaacttattctcatgcctcagcctcccgagtaactgggactacagtgcaccaccaccacacccagctaatttttgaatatttagtagagacagggtttcaccatgttggccaggctggtcttgaactcctggcctcaagtgattcacccaccccgcaagtgctgggattacaggtgtggaccaccgtgcacagccctagtgactttttttttagccccttaatcttttctttcctgggtctcttcattgtcagtgtctgctatttactccctacctagtcacccccttcaccagtatattatgtcctttatgttttattttgcaggatcttattttgcttttctattgaatcccctccatctagaatagtactagacatagtaaatattggttgtatgagtgaatcgctgcttttaattatcatcaccattgctctctctacttctggtctatgatccactttgagttaacttttgttatttggtgtgagataggagtataatttcattcttttacatgtggttatacttttgtctcaacactgtttgttaaaaacacaaaaagtattattttcccatttaatcatctttggcctgggcacggtggctcatgcctgtaatcccagcactctggaaggccaaggcagatggatcaatttgaggccaggagttcaagactagccaacatggtgaaactaaaaatacaaaaaattagctgggtatggtggtgcatgtctgtaatcccagctactcgggaggctgaggcacgagaattgcttgagcctaggaggtggaggttgtagtgagctgagattgtgtcactaccctccagcctgggtgatagagtgagtctgtctcaaaaaaaaaaaaaaaaaattaagaaaataaaaatcgtcggccaggcatggtggctcacacctgtaatcccagcactttgggaggcagaggcgggcagatcacgaggtcaggagatggagaccatcctggctaacatggtgaaaccccgtctctactaaaaataaaaaaattagccgggcatggtgctgggcgcctgtagtcccagctgctcgggaggctgaggcaggagaatggcgtgaacccaggaggtggagcttgcagtgagccgagatcgtgccactgcactccagcctgggagacagagcgagactccgtctcaaaaaaaaaaaaaaaaaaaattgtcttggtatttattattgttgaaaatcgcttgatcacagatgtatgtatgagtttatttctgtactgtcaattccattttattgatgtatgtgtctattcttatgctattaccacactttcttgattactatagctttgtggtgaggtgttgagattttaaactaattataagcatcttacatgaactacttaccgtttatatttgattatgcagcatgaaataattatgaatatatcattaaatatgccatattaacttttattaagttttatgtgatcataacagtaagccatatgcatgtaagttcagttttcatagatcattgcttatgtagtttaggtttttgcttatgcagcatccaaaaacaattaggaaactattgcttgtaattcacctgccattactttttaaatggctcttaagggcagttgtgagattatcttttcatggctatttgccttttgagtattctttctacaaaaggaagtaaattaaattgttctttctttctttataatttatagattttgcatgctgaaacttctcaaccagaagaaagggccttcacagtgtcctttatgtaagaatgatataaccaaaaggtatataatttggtaatgatgctaggttggaagcaaccacagtaggaaaaagtagaaattatttaataacatagcgttcctataaaaccattcatcagaaaaatttataaaagagtttttagcacacagtaaattatttccaaagttattttcctgaaagttttatgggacatctgccttatacaggtattagaaacttactgcctttctctaatgcttctagtgtaaaaacttgcagacttatgtaaagtagggctgtatcgccgtgcccccattgtctgttaatcttgtttttatatttttgattgtgtttccttttctttttttttttttttttaagacagggtcctgctctgtcactgaggctggagtgcagtggcgtgatctcggctcactgtagcctctgtctcccagcctcttcctgccttagcctcccaaatagctgggactacaggcacacgctaccatgcccggccaatttttgtattttttgtagagatgaggttttaccatgttgcccaggctggtaactcctgagctcaggtgatctgcccacctcggcctcccaaagtgctggggttcacaggtgtgtgtttatttctatctaattatttacacaaacacaatgtatttatatattgtgtatctcttctgctacaatgtaaattctatgagagtagtaattttgtctgtctcaacactgtttttcctaagtttggtacatagtaggcactcagatgcttaaaggaatgaatgaattgtgctttaattccactttactaaacccaaatctccctttggacattgttatctatgtgttttcaaagaagtataatcataatttgacagaaatccttgagaggcagaactaagtgagggattgggcagggttcagatgttaagaacagtaagctcagcagggtgtgattgctcatgcctataaccctagcactctaggaggctgaggtgggatgattgcttgaggccaggagtttgaaatcagcctgggcaacatagtgagaccccatcactaccaacaaaataaataaataaatgtacatggtggcatatgcccatagtcctagctacttgggaggctatagtgggaggatagcttgagtacagaagtctgaggctgcagtgagctatgattgtggcactgcatgctagcctgggcaatagagcaagaccctgtctctaaattaaacaaaaaaaaaagtactctagttttctatgcaatgcattatatctgctgtggatttagggcagtattatatcagataattttaggcatttggtaggcttaaatgaatgacaaaaagttactaaatcactgccatcacacggtttatacagatgtcaatgatgtattgattatagaggttttctactgttgctgcatcttatttttatttgtttacatgtcttttcttattttagtgtccttaaaaggttgataatcacttgctgagtgtgtttctcaaacaatttaatttcaggagcctacaagaaagtacgagatttagtcaacttgttgaagagctattgaaaatcatttgtgcttttcagcttgacacaggtttggagtgtaagtgttgaatatcccaagaatgcaactcaagtgctgtccatgaaaactcaggaagtttgcacaattactttctatgacgtggtgataagaccttttagtctaggttaattttagttctgtatctgtaatctatttttaaaaaattactcccactggtctcacaccttattttatcaatcgtaaggtgcacatttttcacatcttaacatctctgaaattgggaacattttactattgagggtgtgtcatttgtttaatttgtgtgctttctttcttagtgatacacgaaataatagtgccacttacattgttggtgtcttagctttagtgaaatacagtattgataggcaaatttcttagtgttaaggtagaaaacaaggactctaaataactttgatggtctgtgtatttgtttttgtttcctaggagtaaaatttccagttgattttttaaaatttgatttttaaaaaaaatcacaggtaaccttaatgcattgtcttaacacaacaaagagcatacatagggtttctcttggtttctttgattataattcatacatttttctctaactgcaaacataatgttttcccttgtattttacagatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttggtaaaaccatttgttttcttcttcttcttcttcttcttttcttttttttttcttttttttttttgagatggagtcttgctctgtggcccaggctagaagcagtcctcctgccttagcccccttagtagctgggattacaggcacgcgccaccatgccaggctaatttttgtatttttagtagagacggggtttcatcatgttggccaggctggtctcgaactcctaacctcaggtgatccacccacctcggctccccaaattgctgggattacaggtgtgagccactgtgcccggccggtaaaaccattttcatttattctggcaacatctctttattgagcattgtgaatatgttagtgaatgtgctagatgctcatagatttatataaaaagttagtgaagaaggaaagatggtatattaagtggttagacaagtgttctaatcagttagagttcagagaaggtcagggtacctgatataatcaagagagagaccttacagccaggtgaggtgaatgtacctataatcccagctacttaggaggctgaaatgggaggatcacttgagtccaggtttgagaccagcccaggcaacatagcaagatccccatcagatacaccaaaaagacagatttcttttttttttttttttttgagacagagtctcgctctgtcgcccaggctggagcgcagtgacacgatgtcagctcactgcaacctccgcctcccaggttcaagtgattctcctgcctcagcctcctgagtagttgggactacaggggtacgacaccagacctggctaatttttgtaattttagtagagtcggggtttcaccatattggtcaggctggtctcgaactcctgacctcaggtgatccaccctccttggcctcccagagtgctgggattacaggcgtgagccaccaagcccggccaaaaaagagagctcttataggcccttccttgctttggagctttatctgctctgtgatgcttatctaaaatagccataaggtcactgatatttttaagcatttggaaattacttcagctgggtgccatggctcatgcctataatcccaaccctttgggaggctgaggtaggaggtcctttgagcccagcttgggcaacacagtgagacactgtctctgcaattaaaaaaaaaaaaaaagtagctgggtgccgtggctcacgcctgtaattccagcactaggaggcttgaggattgcctgagctcaggagttcaagaccagtttgggcaacatagcaagtccttgtctatattaaaagtttttttaaattatctgggcatggtggtgtgtgcctgtagtcccagctacttgggaagctgagacagaaggatcacttgagtccaggagatgtagactacagtgagctatgatcactccactgcacttcagcgtgggcggcaaagcaagatctagttgcaaaaaaaaaaagaactggctgggtgcggcggctaacacctgcaatcccagcaccttgggaggctgaggccagtggatcatgaggtcaggagattgagaccaccctggccaacatggtgaaacccggtctctactaaaaatacaaaaattagctgggtgtggtggcacgtgcctgtaatcccagctactccagaggctgaggatggagaatcacttgaacctgagagtcggaggttgcagtgagccgagattgcgccactgcactccagcctggcgacagagcgagactccgtctcaaaaaaaaaaaaaaaaaagcttcacgcctgtaatcccagcactttgggaggccgagtcaagtggatcacgaggtgtggagatcaagactatcctggctcacatggtgaaagcccgtctctactaaaaacacagaaaaattagctgagcgtgatggcggactcctgtagtcccagctactcgggaggctgaggcaggagaatagcatgaacccgggaggtggagcttgcagtgagccgagatcccgccactgcgatccagcctgggcgacagagtgagactctgtctcaaaaaaaaaacaaaaaaacttagctgggcgtggtggtatgcacctgtggtcctagctacttgggaggctgaggctggagcattgctttaacatagagagtcaaggctgcagttgagctatgactgtgccactggactccagcgcaggtgactgagaccctatcttttaaaaaaagggaaaattacttgaacttaaaaggtgtaattgttaaagaaaatgtagtgatttgctctgttgttacttatatgtgcatgaatgatggagatcttaaaaagtaatcattctggggctgggcgtagtagcttgcacctgtaatcccagcacttcgggaggctgaggcaggcagataatttgaggtcaggagtttgagaccagcctggccaacatggtgaaacccatctctactaaaaatacaaaaattagctgggtgtggtggcacgtacctgtaatcccagctactcgggaggcggaggcacaagaattgcttgaacctaggacgcggaggttgcagcgagccaagatcgcgccactgcactccagcctgggccgtagagtgagactctgtctcaaaaaagaaaaaaaagtaattgttctagctgggcgcagtggctcttgcctgtaatcccagcactttgggaggccaaggcgggtggatctcgagtcctagagttcaagaccagcctaggcaatgtggtgaaaccccatcgctacaaaaaatacaaaaattagccaggcatggtggcgtgcgcatgtagtcccagctccttgggaggctgaggtgggaggatcacttgaacccaggagacagaggttgcagtgaaccgagatcacgccaccacgctccagcctgggcaacagaacaagactctgtctaaaaaaatacaaataaaataaaagtagttctcacagtaccagcattcatttttcaaaagatatagagctaaaaaggaaggaaaaaaaaagtaatgttgggcttttaaatactcgttcctatactaaatgttcttaggagtgctggggttttattgtcatcatttatcctttttaaaaatgttattggccaggcacggtggctcatggctgtaatcccagcactttgggaggccgaggcaggcagatcacctgaggtcaggagtgtgagaccagcctggccaacatggcgaaacctgtctctactaaaaatacaaaaattaactaggcgtggtggtgtacgcctgtagtcccagctactcgggaggctgaggcaggagaatcaactgaaccagggaggtggaggttgcagtgtgccgagatcacgccactgcactctagcctggcaacagagcaagattctgtctcaaaaaaaaaaaacatatatacacatatatcccaaagtgctgggattacatatatatatatatatatatatcatatctatatatatatatatgtaatatatatgttatatatatattacatatatatatgttatatatatgttatatatatataatatatatatgttatatatatgttatatatatatatacacacacacacacatatatatgtatatatatatacacacacacacacatattagccaggcatagttgcacacgcttgtagacccagctactcaggaggctgaggcaggagaatctcttgaacttaggaggcggaggttgcagtgagctgagattgcgccactgcactccagcctgggtgacagagcaggactctgtacaccccccaaaacaaaaaaaaaagttatcagatgtgattggaatgtatatcaagtatcagcttcaaaatatgctatattaatacttcaaaaattacacaaataatacataatcaggtttgaaaaatttaagacaacagaaaaaaaaattcaaatcacacatatcccacacattttattattactactactattattttgtagagactgggtctcactctgttgcttatgctggtcttgaactcctggcctcaagcagtcctgctccagcctcccaaagtgctgggattataggcatgagctaccgctcccagccccagacattttagtgtgtaaattcctgggcattttttccaggcatcatacatgttagctgactgatgatggtcaatttattttgtccatggtgtcaagtttctcttcaggaggaaaagcacagaactggccaataattgcttgactgttctttaccatactgtttagcaggaaaccagtctcagtgtccaactctctaaccttggaactgtgagaactctgaggacaaagcagcggatacaacctcaaaagacgtctgtctacattgaattgggtaagggtctcaggttttttaagtatttaataataattgctggattccttatcttatagttttgccaaaaatcttggtcataatttgtatttgtggtaggcagctttgggaagtgaattttatgagccctatggtgagttataaaaaatgtaaaagacgcagttcccaccttgaagaatcttactttaaaaagggagcaaaagaggccaggcatggtggctcacacctgtaatcccagcactttgggaggccaaagtgggtggatcacctgaggtcgggagttcgagaccagcctagccaacatggagaaactctgtctgtaccaaaaaataaaaaattagccaggtgtggtggcacataactgtaatcccagctactcgggaggctgaggcaggagaatcacttgaacccgggaggtggaggttgcggtgaaccgagatcgcaccattgcactccagcctgggcaaaaatagcgaaactccatctaaaaaaaaaaaagagagcaaaagaaagaatatctggttttaaatatgtgtaaatatgttttggaaagatggagagtagcaataaggaaaaacatgatggattgctacagtatttagttccaagataaattgtactagatgaggaagccttttaagaagagctgaattgccaggcgcagtgctcacgcctgtaatcccagcactttggaaggccgaggtgggcggatcacctgaggtcgggagttcaagaccagcctgaccaacatggagaaaccccatctctactaaaaaaaaaaaaaaaaaaattagccggggtggtggcttatgcctgaaatcccagctactcaggaggctgaggcaggagaatcgcttgaacccaggaagcagaggttgcagtgagccaagatcgcaccattgcactccagcctaggcaacaagagtgaaactccatctcaaaaaaaaaaaaaaagagctgaatcttggctgggcaggatggctcgtgcctgtaatcctaacgctttggaagaccgaggcagaaggattggttgagtccacgagtttaagaccagcctggccaacataggggaaccctgtctctatttttaaaataataatacatttttggccggtgcggtggctcatgcctgtaatcccaatactttgggaggctgaggcaggtagatcacctgaggtcagagttcgagaccagcctggataacctggtgaaacccctctttactaaaaatacaaaaaaaaaaaaaaattagctgggtgtggtagcacatgcttgtaatcccagctacttgggaggctgaggcaggagaatcgcttgaaccagggaggcggaggttacaatgagccaacactacaccactgcactccagcctgggcaatagagtgagactgcatctcaaaaaaataataatttttaaaaataataaatttttttaagcttataaaaagaaaagttgaggccagcatagtagctcacatctgtaatctcagcagtggcagaggattgcttgaagccaggagtttgagaccagcctgggcaacatagcaagacctcatctctacaaaaaaatttcttttttaaattagctgggtgtggtggtgtgcatctgtagtcccagctactcaggaggcagaggtgagtggatacattgaacccaggagtttgaggctgtagtgagctatgatcatgccactgcactccaacctgggtgacagagcaagacctccaaaaaaaaaaaaaaaagagctgctgagctcagaattcaaactgggctctcaaattggattttcttttagaatatatttataattaaaaaggatagccatcttttgagctcccaggcaccaccatctatttatcataacacttactgttttccccccttatgatcataaattcctagacaacaggcattgtaaaaatagttatagtagttgatatttaggagcacttaactatattccaggcactattgtgcttttcttgtataactcattagatgcttgtcagacctctgagattgttcctattatacttattttacagatgagaaaattaaggcacagagaagttatgaaatttttccaaggtattaaacctagtaagtggctgagccatgattcaaacctaggaagttagatgtcagagcctgtgctttttttttgtttttgtttttgttttcagtagaaacgggggtctcactttgttggccaggctggtcttgaactcctaacctcaaataatccacccatctcggcctcctcaagtgctgggattacaggtgagagccactgtgcctggcgaagcccatgcctttaaccacttctctgtattacatactagcttaactagcattgtacctgccacagtagatgctcagtaaatatttctagttgaatatctgtttttcaacaagtacatttttttaacccttttaattaagaaaacttttattgatttattttttggggggaaattttttaggatctgattcttctgaagataccgttaataaggcaacttattgcaggtgagtcaaagagaacctttgtctatgaagctggtattttcctatttagttaatattaaggattgatgtttctctctttttaaaaatattttaacttttattttaggttcagggatgtatgtgcagtttgttatataggtaaacacacgacttgggatttggtgtatagatttttttcatcatccgggtactaagcataccccacagttttttgtttgctttctttctgaatttctccctcttcccaccttcctccctcaagtaggctggtgtttctccagactagaatcatggtattggaagaaaccttagagatcatctagtttagttctctcattttatagtggaggaaataccctttttgtttgttggatttagttattagcactgtccaaaggaatttaggataacagtagaactctgcacatgcttgcttctagcagattgttctctaagttcctcatatacagtaatattgacacagcagtaattgtgactgatgaaaatgttcaaggacttcattttcaactctttctttcctctgttccttatttccacatatctctcaagctttgtctgtatgttatataataaactacaagcaaccccaactatgttacctaccttccttaggaattattgcttgacccaggtttttttttttttttttttggagacggggtcttgccctgttgccaggatggagtgtagtggcgccatctcggctcactgcaatctccaactccctggttcaagcgattctcctgtctcaatctcacgagtagctgggactacaggtatacaccaccacgcccggttaattgaccattccatttctttctttctctcttttttttttttttttttgagacagagtcttgctctgttgcccaggctggagtacagaggtgtgatctcacctctccgcaacgtctgcctcccaggttgaagccatactcctgcctcagcctctctagtagctgggactacaggcgcgcgccaccacacccggctaatttttgtatttttagtagagatggggtttcaccatgttggccaggctggtcttgaactcatgacctcaagtggtccacccgcctcagcctcccaaagtgctggaattacaggcttgagccaccgtgcccagcaaccatttcatttcaactagaagtttctaaaggagagagcagctttcactaactaaataagattggtcagctttctgtaatcgaaagagctaaaatgtttgatcttggtcatttgacagttctgcatacatgtaactagtgtttcttattaggactctgtcttttccctatagtgtgggagatcaagaattgttacaaatcacccctcaaggaaccagggatgaaatcagtttggattctgcaaaaaagggtaatggcaaagtttgccaacttaacaggcactgaaaagagagtgggtagatacagtactgtaattagattattctgaagaccatttgggacctttacaacccacaaaatctcttggcagagttagagtatcattctctgtcaaatgtcgtggtatggtctgatagatttaaatggtactagactaatgtacctataataagaccttctgtaactgattgttgccctttcgtttttttttttgtttgtttgtttgtttttttttgagatggggtctcactctgttgcccaggctggagtgcagtgatgcaatcttggctcactgcaacctccacctccaaggctcaagctatcctcccacttcagcctcctgagtagctgggactacaggcgcatgccaccacacccggttaattttttgtggttttatagagatggggtttcaccatgttaccgaggctggtctcaaactcctggactcaagcagtctgcccacttcagcctcccaaagtgctgcagttacaggcttgagccactgtgcctggcctgccctttacttttaattggtgtatttgtgtttcatcttttacctactggtttttaaatatagggagtggtaagtctgtagatagaacagagtattaagtagacttaatggccagtaatctttagagtacatcagaaccagttttctgatggccaatctgcttttaattcactcttagacgttagagaaataggtgtggtttctgcatagggaaaattctgaaattaaaaatttaatggatcctaagtggaaataatctaggtaaataggaattaaatgaaagagtatgagctacatcttcagtatacttggtagtttatgaggttagtttctctaatatagccagttggttgatttccacctccaaggtgtatgaagtatgtatttttttaatgacaattcagtttttgagtaccttgttatttttgtatattttcagctgcttgtgaattttctgagacggatgtaacaaatactgaacatcatcaacccagtaataatgatttgaacaccactgagaagcgtgcagctgagaggcatccagaaaagtatcagggtagttctgtttcaaacttgcatgtggagccatgtggcacaaatactcatgccagctcattacagcatgagaacagcagtttattactcactaaagacagaatgaatgtagaaaaggctgaattctgtaataaaagcaaacagcctggcttagcaaggagccaacataacagatgggctggaagtaaggaaacatgtaatgataggcggactcccagcacagaaaaaaaggtagatctgaatgctgatcccctgtgtgagagaaaagaatggaataagcagaaactgccatgctcagagaatcctagagatactgaagatgttccttggataacactaaatagcagcattcagaaagttaatgagtggttttccagaagtgatgaactgttaggttctgatgactcacatgatggggagtctgaatcaaatgccaaagtagctgatgtattggacgttctaaatgaggtagatgaatattctggttcttcagagaaaatagacttactggccagtgatcctcatgaggctttaatatgtaaaagtgaaagagttcactccaaatcagtagagagtaatattgaagacaaaatatttgggaaaacctatcggaagaaggcaagcctccccaacttaagccatgtaactgaaaatctaattataggagcatttgttactgagccacagataatacaagagcgtcccctcacaaataaattaaagcgtaaaaggagacctacatcaggccttcatcctgaggattttatcaagaaagcagatttggcagttcaaaagactcctgaaatgataaatcagggaactaaccaaacggagcagaatggtcaagtgatgaatattactaatagtggtcatgagaataaaacaaaaggtgattctattcagaatgagaaaaatcctaacccaatagaatcactcgaaaaagaatctgctttcaaaacgaaagctgaacctataagcagcagtataagcaatatggaactcgaattaaatatccacaattcaaaagcacctaaaaagaataggctgaggaggaagtcttctaccaggcatattcatgcgcttgaactagtagtcagtagaaatctaagcccacctaattgtactgaattgcaaattgatagttgttctagcagtgaagagataaagaaaaaaaagtacaaccaaatgccagtcaggcacagcagaaacctacaactcatggaaggtaaagaacctgcaactggagccaagaagagtaacaagccaaatgaacagacaagtaaaagacatgacagcgatactttcccagagctgaagttaacaaatgcacctggttcttttactaagtgttcaaataccagtgaacttaaagaatttgtcaatcctagccttccaagagaagaaaaagaagagaaactagaaacagttaaagtgtctaataatgctgaagaccccaaagatctcatgttaagtggagaaagggttttgcaaactgaaagatctgtagagagtagcagtatttcattggtacctggtactgattatggcactcaggaaagtatctcgttactggaagttagcactctagggaaggcaaaaacagaaccaaataaatgtgtgagtcagtgtgcagcatttgaaaaccccaagggactaattcatggttgttccaaagataatagaaatgacacagaaggctttaagtatccattgggacatgaagttaaccacagtcgggaaacaagcatagaaatggaagaaagtgaacttgatgctcagtatttgcagaatacattcaaggtttcaaagcgccagtcatttgctccgttttcaaatccaggaaatgcagaagaggaatgtgcaacattctctgcccactctgggtccttaaagaaacaaagtccaaaagtcacttttgaatgtgaacaaaaggaagaaaatcaaggaaagaatgagtctaatatcaagcctgtacagacagttaatatcactgcaggctttcctgtggttggtcagaaagataagccagttgataatgccaaatgtagtatcaaaggaggctctaggttttgtctatcatctcagttcagaggcaacgaaactggactcattactccaaataaacatggacttttacaaaacccatatcgtataccaccactttttcccatcaagtcatttgttaaaactaaatgtaagaaaaatctgctagaggaaaactttgaggaacattcaatgtcacctgaaagagaaatgggaaatgagaacattccaagtacagtgagcacaattagccgtaataacattagagaaaatgtttttaaagaagccagctcaagcaatattaatgaagtaggttccagtactaatgaagtgggctccagtattaatgaaataggttccagtgatgaaaacattcaagcagaactaggtagaaacagagggccaaaattgaatgctatgcttagattaggggttttgcaacctgaggtctataaacaaagtcttcctggaagtaattgtaagcatcctgaaataaaaaagcaagaatatgaagaagtagttcagactgttaatacagatttctctccatatctgatttcagataacttagaacagcctatgggaagtagtcatgcatctcaggtttgttctgagacacctgatgacctgttagatgatggtgaaataaaggaagatactagttttgctgaaaatgacattaaggaaagttctgctgtttttagcaaaagcgtccagaaaggagagcttagcaggagtcctagccctttcacccatacacatttggctcagggttaccgaagaggggccaagaaattagagtcctcagaagagaacttatctagtgaggatgaagagcttccctgcttccaacacttgttatttggtaaagtaaacaatataccttctcagtctactaggcatagcaccgttgctaccgagtgtctgtctaagaacacagaggagaatttattatcattgaagaatagcttaaatgactgcagtaaccaggtaatattggcaaaggcatctcaggaacatcaccttagtgaggaaacaaaatgttctgctagcttgttttcttcacagtgcagtgaattggaagacttgactgcaaatacaaacacccaggatcctttcttgattggttcttccaaacaaatgaggcatcagtctgaaagccagggagttggtctgagtgacaaggaattggtttcagatgatgaagaaagaggaacgggcttggaagaaaataatcaagaagagcaaagcatggattcaaacttaggtattggaaccaggtttttgtgtttgccccagtctatttatagaagtgagctaaatgtttatgcttttggggagcacattttacaaatttccaagtatagttaaaggaactgcttcttaaacttgaaacatgttcctcctaaggtgcttttcatagaaaaaagtccttcacacagctaggacgtcatctttgactgaatgagctttaacatcctaattactggtggacttacttctggtttcattttataaaagcaaatccaggtgtcccaaagcaaggaatttaatcattttgtgtgacatgaaagtaaatccagtcctgccaatgagaagaaaaagacacagcaagttgcagcgtttatagtctgcttttacatctgaacctctgtttttgttatttaaggtgaagcagcatctgggtgtgagagtgaaacaagcgtctctgaagactgctcagggctatcctctcagagtgacattttaaccactcaggtaaaaagcgtgtgtgtgtgtgcacatgcgtgtgtgtggtgtcctttgcattcagtagtatgtatcccacattcttaggtttgctgacatcatctctttgaattaatggcacaattgtttgtggttcattgtctccttaaattagactgtaagcaccttgatggaactcatactaccttttatttcacacacacgcacacgcgcacacacagcctacacatacactgcctagctcattgtagcatactaaatactgattttaatgaataagctaaaccttcgaaacccatttgctaatcccagcactttgggaggccaaggtgggtggatcacctcaggtcagaagtttgagaccagcctggccaacatggtgaaaccccacatctactaaaaatacaaaaattagctgggcgtggtggccaatgccttgtaatcccagctattctggaggctgagacaggagaatcgcctgaacctgggaggcggaggttgcactgagctgggattgtaccactgcactccagcctgggtgacaaagtgagactccatctcaaaaacaaacaaacaaaaacacatcatttcccctatagcaaaaacatgacggcacttactgtatcaagagaggtgagaaaaaggagccacagcaggatgattcaagggactctgcatagctccattttaagaatatgcctactgcaggtcagagaaggtaagcaaactgcctaaggccacacagccaggtacagaactctcaccaatattattgccagcaatcgcaattttggtgtttattcttggtaccaagttggagactatagggttctcttcctaatagagaccatctagcctttcactgttttgtggatacttctttctcttcttctttttttttttcccttttaaaatctagttatttttttctttttggtttctttgacacagggtctcttactctgttacccaggctggaatggagtagtgcagtcatggttcactgtagctttgacttcctgggctcaagcgatcctcctacctcagcttcccgagtagctgggaccacaggcgcccaccaacacctccagctaatttttaagtttttactagagacaacatctcactatgttgcccaggctggtctcaaaatcctgggctcaagtgatcccacctcagccttccaaaatgctgggattacaggtgtgtgcaccacgcctggcctattttttttttaattgctcataaatcatcttttttctttaaaaaaaagaaagatgggaggctaaagcaggagaatcacttgaacccaggaggcggaggttgcagtgagctgagatcatgctgctgctctccagcctgggcaacaagagtgaaactccatctcaaaaaaaaaaaaaaagaaagtacacaattttactttctggacctaatggtcaaggccaataatttggtcacctatgaaataaataaaagctttaccatatatatgaccatttgataatgtaatatgaaatgtttatgtactaaaggcagaatagtctagaaaaaacattctgtatcacaacgtctaaaaatgaatatcatcttcatcatagaaccaggctctttctcctaatttttttttttgagatggagttttgctctgtcacccaggctggaatgcagtggcacaattttggctcactgcaaccttcagctcccaggttcaggatcaagtgattttcgtgcttcagccttctaagtagctgggattacaggtgactgccaccacacccagctcatttttttgtatttttttagtagagagagggtttcaccatgttggccaggctcgtctcgaactcctgacctcaaataatccacccgtctcagcctcccaaagtgctgagattacaggcgtgagccaccaggcctggcctcctaatttttatttgtagaagtggcaccaaaattttccaagttctcatgcaaaaattcaggctcatctcagtttatttttttcatttatttatctcccactaaattgacaacttctaataattaggttggttctttgtattcccagcacagggttctatgcagaatacacacacagcagttgctggcaataatattggtgagagttctgtactgggctatgtgatcttagacagtttgcttatgttctctgacctgccgtaggcacattcttaaaatgaagctgttcagaccccctcgattcatcctgctgtggcttctttttcccacctaaatcttaaatacccttttagctgctagtaagtgaatgatgtttttttatgaactttctgaagtcagattagatgaagttgagaaaagcctgatattcttataaagttatatatgtgcatcatagaaaacttagaaaatacagataaacaaaaatcatccatggacgaaccttgaagacattgtgttaactgaaataaaccggacaccaaaggacacatgttatatgcttccacttatatgagatacctagaatagttacatttggttactctgggtacattgcctatagataagccttgctccacaaggagcagttaaaaaaaaaaaaaagataaattcataggatggaaggtagaatagtggttactagggacttggggagggggaaatggggagttactgtttgatgagtgcagatttcagtttgggatgatgaaaaagttctggagatagatagtggcaatggtaacacaacagtgtgaaaataatgccactgaactgtacacttaaaatgattaaaatgataagttaattgtaatttgtgttatccagaaatggttagcaatttattggtgtatattcttttagtattcctgtgtgtgcacaggggtgcttgtatatactttatctttaaaatatatccaggaagctaggcacagtggcttacacctgtaatcccagcactttgggagggtgaggcaggaagattgcctgagccccggaggtcaaggctgcagtgagttgtgatcacgctactgcactctgttctgggcaacccctgtctgggaaaaaaaaaaaaattagtgaggcttagtggtgcacacctgtagtctcagctacttgagtggctggggtaggattgcttgatcccagcaagttgaggccgtggtgagccatgatggtgccactgcactccatcctgggtgatatggtgagaccctgtctcaaaaacaagaaatccagataattctgtgcattataatctagcttttactggatcattaaaattcttttttctttttttttttttttttctgagatggagtttcactcttgttgcccaggctggagtgcagtggtgtgaccttggctcaccgcatcctctgcctcccgggttcatgcgattctcctgcctcagcctcccgagtagctgggattacaggcatgtgccaccatgcccagctaactttgtatttttagtagagacagggtttctccatgttgaccaggctggtctcaaactcctggcctcaagtgatccacccacctcggcctcccaaagtgctggggttacaggcgtgagccaccgcactcagcctgggtcgttaaaattcttaagtgacttcatttttaattactatatgggattctatctttccagtgtatcatgatttatttgacctattgctgaatgttggaggtttcagggtaagaggcacagtttgctattatgtacatcactatagtggcatcctgatagctaaatatttgcctacatccctgattatttccttagtctaaattactgggactaggattttggtgtttgatacatgttactaaattgttttttagaaagattaaaccagtttatgctcttccagcccctgtggtatatgatagttcccattttcctgtaccttgccaacactgggtgatatccagttttaaaatctaaatcttgcattgctatgagaactacaattagagaaggcttatcttctactgcccattctctgtacagagcaaatccctctagacctgaagccccttggagttgtcaagaaacctttgagatgactccccactctgtatctgagctgtcaccagtattctccacttcttcaggattgccatggcaactaaattgatgaaaagatttaggaggccttttctctctttgcaattcctatgatcctttttgaatgtgggtttgggactctgtcaatatacccatcatctaattctgtccattgtgttttaaagtttaaggttgcaatttctgattacatctgccttagccatactgtattatatttgacattcaatatacaatgtccttgtttttctgtatttctaatcttattcccagagatgtgtctatttgttcaggattcattttgcaacgtgtttttactaagcatctacccaaaaccgttgaagtcagatttcaggctgtcttacgtctaaagtagcacaggcaggaaaaactattgaagtgggatttttttttccctttttgtactgaaccgagaaaaagtatatagatgatagagaattcctaatttggtatcattgatatctgggtttttgtttgtttttacagaagactgattaactatacttatttattaatttatcttctcattaataaacacttgctgagtgcttactgtctgctaggcattagggagacaaatatgattaagggaagcttcctcctatcaaggtcatgtgttccatttgggtatactaatgcattagcaatgtaaatcaagtagtgagagatcatctgttcccgataggagatggattattggtggggacttctgtgtgtgtgtgtgtgtgtgtgtgtgtgtgtgtgtgtgtatgtatgtgataaaataaatataggaaatgttaattatagattctaagtagtagatatataaacactcattgcaaagttgcttcaagttttctgtatatttgaaaatattcacaacatgtcgacaaaactagcatgataaagccactatttgtgctaagacttcagcttgtatctggattaggcttattatgtagtagtaggaacattagaaatagttttaactcattaaatacacatgttttatgggaaggttttatatatatatttatatgtaatgaatgtgaacaaacaagggtcagatatacactctgcttccctccagaccagttccggctgctctgctgcacatttcaggagtcttattagaattagccacattctgcccacttgcccttacttctcatatttcacaactcctcctggtggggacttaaggagacattcaaactaggccttgaaagatgagaatttttccaagtggaaaaagaggagtggcagcaagtaaggtaaaggtacagagtcatggaattcccaggaaacgtaaagttgtcatgtgttataggaaaacaacttgtgtgaggggtgttgggagaaatgagagataataccagggtataaagggccttttgaatgctatgttgaggaattttatcctaatggcagtaatgactaacaattatatagtgttcaaaaagtataaatcagcagtggtataccactaagggtttttttcttttctttttttttttgagacagagttttgctctgttgcccaggctggagtgccgtggcacgatctcagctcactgcacttccgccacctgggttcaagtgattcttctgcctcagccagtgtttcactgtgatggccaggatggagcactaagggtctttatggaagaaaaagacatgataaacaaggcttttagggaacttctacagtaatgtagctgtattaaaagtagagatcagagcagcatagtagaagtagaaggctagagctaattgaaggagcacttcagaattagaatcaagaagtcttagaaacctattggttttattctccctaatgtatttggccacttacctgctggggaatttgtctaagttataaaaaataattcctttgggaaacccaaaggaaagttatctattaataattaccccactactttttctgatttatgtaatggccacgtagaggttagatgtgatggttgtgacagtagtgactaatacagcctgtgaagcattttggtcagatatctatgtgctttcattccaggttgactgaggcaagactttggctagggtttgatcagtgatgtaactactcacgagtaccacgtggtggcaatggcattgctgcagaccttggcagcaaagcagtgttagagtagcagtagaaacctttgtgaagctaggaatacattttctggtcataaaaacctcctgaaaattgtgaactcagtgtagcaggagaaagaagatggcttgtttttagtaaagggcaaagtcatttttaaggatcagaagaagaaacggagagtgaaacaatgtgttcctgccctactcccccactggactttttggcaaccattgctgttccttctaaaagtgatttttaaacatgtatattttgaagccaggcacagtgactcacgtctgtaatcccagcactttgggaggccgaggcgggcagatcacctgaggtcaggagttcaagaccagcctggccaacatggtgaaaccccgtctctactaaaaatacaaaaattaggccaggtgtggtggctcacgcctgtaatcccagcactttgggaggccgaggcgggaggatcatgtggtcaggagatccagaccatcctggctaacacggtgaaacaccatttctactaaaaatacaaaaaattagctgggcatggtggcgggcgcctgtaatcccagctactcaggaggctgaagcagaagaatggcttgaacctgggaggcggagcttgcagtgaaccaagattgcgccactgcactccagcctgggcaacaaagtgagactccgtctcaaaaaaaaaaaaaaaaattagtcgggcatggtaacaggtgcctgtaatcccagctacttgagaggctgaggcagggagaattgcttgaaccaggtaggcggaggttgcagtgagccaagatcgcaccactgcactccagcctggggcaacagagcaagactgtctcaaaaaaaataaataaataaaataaattcttaagaaggatattttggaaaactccttacatacctaaattctttgtttatcaaatacttggacttagcacactcttctttgaaatggaccaataaacaacaggagcccataagcaaaaagaactcattattttaaaaacagtaactatccttacaggctttctcagggctctttctgttggatccttccctctcacaggtccttgctaatgatctctaggtggacacattctagatgagatgtccctgtctagaatggcagcaccatgagggctatatcctcagtactaggacagcgcctggtgcttaatagatagtaaatagttgtctaattaactgagcaaacagatagattcatgaattagctttttgctttttctgttagaaactaaaggttcaggtcaggcacaatggcgcatgtctctaatcccagcactttgggaggccgaggcgggctgatcacttgaggtcaggagttcaagaccagcctggccaacatagtaaaaccctgtttctacaaaaattaccaaaattagccgggcgtcttggcaagcacctgtaatgccagctacttgagaggctgaggtgggagaatcgcttgaacctgggaggaagaggttgcagtgagccgagatggtgccaacctgggtgacagagggagacttaaaaaaaaaaagaaagaaagaaagaaaagaaactaaaggttcaaagaatcccagaaaaggaagagtcctcacaagccagtaatctaggcaggattactgatagtatttttatatttgttgtatttttataaaatgccatagatagagggcttttttcaacattacatcagtctaaaaatcacacatttttatatgaactaacctaaatgtctgatgaatctcacaacaccaagtctttgaaatgtgcccatataaataaaatgttaacagattcatgctaattttaaatatcgatagtgtttaaatgccttaattattttttcactccctagctttaaaagaaaataaccaacttcaaaaggacatcacaataacatcaagtctatttgggggaatttgaggattttttccctcactaacatcatttggaaataatttcatgggcattaattgcatgaatgtggttagattaaaaggtgttcagctagaacttgtagttccatactaggtgatttcaattcctgtgctaaaattaatttgtatgatatattttcatttaatggaaagcttctcaaagtatttcattttcttggtgccatttatcgtttttgaagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaaggtgtgtattgttggccaaacactgatatcttaagcaaaattctttccttcccctttatctccttctgaagagtaaggacctagctccaacattttatgatccttgctcagcacatgggtaattatggagccttggttcttgtccctgctcacaactaatataccagtcagagggacccaaggcagtcattcatgttgtcatctgagtacctacaacaagtagatgctatggggagcccatggaagatacatggtatacaacatagctcttgctctattggaagctaagtggaatgggagaaattggtgacaggcaaccccataatttcagaaagctatgaaaaagtactcagacatattccttataacactggtgtcacatcacaaagacctatttaatgtgcttctgatttatagggagagacatcctatacttcaggaactgcactttgatccacagaaagcctagtgatgtagagctcctgttagttcaaaaggaaaagaaaagaacaacacagaaagcctaattatgcaatagagtcaagtgctttatagcaatgttacagttatcaaaaaaaatccagatggacctctgagaggatgccattggagtaaccaggcagatgcagttgatcagagctgacttcctataagaagtgagcactgagctgaggaataatggcataaatgaaggaaagtgagatggaaatttgagtttttaattggaaagacaatacatcaggcagatttttaaataggggcaaacaaacagacacataggagatgctaggcatggggtccccactaggatgctgcttagaaacatgcaggggtggtgagtactcccaaagtacacttcattcctagctcagtgattcttatctgagtgttaaagttccttcttcagcaccccgttccacagtccaactgggaactttaagacctttcttggagtctttctaggaactcaagtctgctacttatacagaacagtggctttggtccccagttgtgccttgcagtatttttgtgttcaggaagaaacagtagctcttggataaagaagctagctagaaactctgttgctatggcagtgcttcaaaatgtatttccttaaatgctttctttgtaactatcttcatttagttcatctctcagataatgagagatcagagtcccatccccagtataatactcttctttagggtactttcaccatcttcagtctaaacacagactagactttcaattataatgtgtaagatttaaaatgttattattgtgtgactttgaatatctgtgtaaatctactatctcctctttggtatatacgtgtgtttatttttttctggagatctgtaactgaaatgcttaatttctgaattgttttggatatcacaacttaataccaacataagttttgagcctttttctccctaaatctggtgtgagtctaactgaaactcaaatgaactttttaaaaataattttttcttttctttaattttttttttaagtagagacagggacgcactgttaactaggctggtcttgaactcctgatcttgagccatcctccccgacctgagcctcaccttatagagagggtcttgctctgttgcccaagctggagggcagtggcataatcacagctcactgcagcctctcgacctcctcaagcgatcctcctgccttagcctcccaagtagctgggactataggcgtccaccaccatacccagctaatttttttttttattttttgtagagacaaggtctccctatgttgcccaagttggtctcaaactcctggactcaagcagtcctctcacctcagggtcccaaagtgctggggttacaggtgtgagccatggcacctggccagaacttctagtaaaaagaatattgttgccgggtacggtggctcacgcctgtaaccccagcactttgggaggccaaggcaggcgaatcacctgaggtcgggagctcgagaccagcctgaccaacatggagaaaccacatctctactaaaactacaaaaaattagccgggcgtggtggcacatgcctgtaatcccagctacttgggagctacggtgcctggcctagtttattatttcttaatatctgttgtcttccagtgtcttccttaattcttcacaataccctgtacaatgcttagcacacagtgggcagtctgtaagtttattaaatgtttggtgtggcccatacttcctatccacaaagaatgtaacatgttaagacatctagatgagggaatgatttaagaggaactacaataatattctgaaacttggactctggatctctgcatttagactttcctaaaccagccagcaagtagatcatcatgtcacaaggcttaggttgggcttgctgttcagagaatgaattaaggattaaggagaaaaaaaagcagaaaggttttgctctgtttttcaggttctattgagttgttaacttctaacaagttatcttatttgcttcattgcatgaggcccattgtagtaagaagaggaatttatatgctaaatgttctggtgatagaatgacttttctttttttttacagtccaaaggtcttttttttttttttttaacacctattatgccatgaattcatagggaataggttccagctgctcaggctccttcccattggttctcacaaagtgtgcttctctgggtggagcaggctggtgcttcagttgaacccacgtacctttctctttggcttctttctttttctgatcattttccttcacgcgtttcaggaagctgtcttggctcttagagtgtttaatgtgctcaatacgcacattaattctcttggcaagaatcttgcccttaacttgtttacagcgatgccaacagcatgctgggtcacgttgtagacttttccagttttgccatggtaacacttgtggggcattcctttttgaacagtacccgttcccttgatgtctacaatttcacctttcttacagattcgcatatacatggccaaaggaacaactccatgttttctaaaaggcctagagaacatatatcaggtgcctctcctctttccctttgtgttcgtcattttggcaaattactgaaagatggtggttctggccaaaaggaggaatgactttttaatagctgtgtttgtatctgagccttccctctgcctttcattttttttgttttgttttgttttgtttttgtttgagatgaagtttcacttttgttgcccaggctggagtgcaatggtgtgatttcggctcattacaatgtccgcctcagcctcctgggtagctgggattacaggcacccgccaccacgcccagctaatttttgtatttttagtagagacagggtttcaccatgttggccgggctggtctcaaactcctgacctcaggtgatctgtccacctcggcctctcaaagtgctgggattgtaggcgtgagccacatcacctggccacttttttaactctttccaatggttaattccgtttgatatggttccttggaacttgcacattaccctttatcaattatcaccctgtattgggggtggggaggatgatacctctcttcatagttagatcctacttactttcaacagagttcttaacaatcctagaaactcacaggtccagaaaagacaagcataaaggaaactataaataatgcatttgaagactaactcaggaaatcaatgattatttccccccaggctacccagtgtcttaaaaaaacagtttaattaatacaatcttttgtttcaattttctacctatatttatggcttttagcttttctaataaaagctcaaaatgaattacagtcatcagtgactttttaatgaatagaagacttttgcaatttttaactatttgtttttacttattaaatatttccgccttggccaggcatggtggctcacgcctataatcccagcactgtgagatgccaaggcaggaggatcacttgagtttaagagttctagaccaggctgggtatggtggctcatgcctataatcccagcactttgtgaggccaaggttggcggatcacctgaggtcaggagtttaagaccagcctggccaacatggtaaaaccccatctctacaaaaaatacaaaaattagccaaggggtggtggtgggcacctataatcccatcttcttgggaggctaaggcaggagaatcgcttgaacctggaggcagaggttgcagtgagccgagatcatgccactgtattccagcctgggtaacagagcaagactctgtctcaaaaaaaaaaaaaagtttgaaaccagcctggtcaacacagcaagacacccatctcgttgaaaaataacggtcgggcgcagtggctcacgcctgtaatcccatcactttgggaggccgaggcaggcagatcacctgaggtcgggagttcgagaccagcgtgaccaacatggagaaaccccatctctactaaaaatacaaaattagttgggcgaggtggtgcatacctgtaatcccaactacttgggaggctgaggcaggagaacagcttgaacctgggaggcagagaggttgtggtgagccaagatcatgccattgcactgcagcctgggcaacaagagcaaaactccatctcaaaaaaaataaataaataaaaataaataaataagtacttctgcctttaagccacttcctagaaggcagtggcacaaagtgatacatttggaggagtaaatatattacaaaatgaattaggctgggcgcagtggctcatgtctgtaatccccgcactttgggaggccaaggcgggtggatcacttgaggtcaggagttcgagactagcctgatcaacagggtaaaatcccatctctactaaaaataccaaaaaaactagctgggcgtggtggcaggcacctgtaatgtcagctactaggaaggctgaggcaggagaatcgcttgaacccaggaggtggaggttgcagtgagccaagattgcaccattgcacttcagcttgggcaacagagtgagactccgtctcaaaaaaaaaaaaagaactaacatgccagaactttgccttcagtatgttttgtgatttttcccttcttgtgccatttcatcattagttccatgtattatttaagatttcttatcaaccagcaccttgggatttttttgtgtatgtgttggtttagggggtttatttgtttttttcttttttttcggtaattgaaaatgtgaagcaaaatgtcacctgttttttctttcatgtctgacactcatgtcttgtttacccccgacatgcagaagctgaaatccccatttcatacagtcttcaatgtggaggcagtagggatggagaaaataatgtactttgtgctctccggtactctttctttcctattgtctgaggggatttgggcataatttattttgctgcagagataaaaatttgttatatatattttttatcattcagggccaaggaatatagattttttttttcagccttgtctcagctgggtgtctttatttactctgtcttaaagtgttccttttattatcattattattttttaatcattgaattccatttggtgctagcatctgtctgttgcattgcttgtgtttataaaattctgcctgatatacttgtttaaaaaccaatttgtgtatcatagattgatgcttttgaaaaaaatcagtattctaacctgaattatcactatcagaacaaagcagtaaagtagatttgttttctcattccatttaaagcagtattaacttcacagaaaagtagtgaataccctataagccagaatccagaaggcctttctgctgacaagtttgaggtgtctgcagatagttctaccagtaaaaataaagaaccaggagtggaaaggtaagaaacatcaatgtaaagatgctgtggtatctgacatctttatttatattgaactctgattgttaatttttttcaccatactttctccagttttttgcatacaggcatttatacacttttattgctctaggatacttcttttgtttaatcctatataggttttttgaacctataacataagctacaacatgagaaatgtgcggttagatagatatgtcccttctgaaggtcagaaaaaaatataatggaggtaaaacctgaacaagcttggaaactgatggtagacttcttcaaggcagcccttgccctaattaaaattcttgtctttctagaaaaagtctagctgttgatttaccacagaaaataataataataattactattattattattttttgagacagggtcgccctgtgtcacctagattgcagtggtgcagtcatggctcactgcatcctccgtttttcaggctcaagcaatcctcccaccttagcctcctgagtagctgggtccacaagcatgcgccacccacacccactaagtttttgtatttttggtagagatggagttttaccttgttgcccaggctggtctcaaattcctggactcaagtagtccgcccgccttgccctcccaaagccagaaaacatttagaatatctttcagagatgtgtatttacaccactattaacacagggctgtatagcagtccagtactggactatgtagtccagtactattcttttccttactggagggccaggcgtggtggcaggtgcctgtaatcccagctactcaggaggctgaggcaggagaattgcttgaacctgggaggcagaggttgcagtgagctgggaccgtgccattgcactccagcctgggcgacagagcaagactccgtctcaaaacaaaaaaaaaaagagagagagagcagtaattcaggtctcacccatcttcaatccagggggcctagccttagtatttgacccatagtaagcacccaataattgtttaaattaattaacctctgaggccctttaaatctgttgataagtatcttattttgcaaagtcctaagcacttggaagagcagaggaactatttactgggtgtgtatgcttttctaacaatattttatagctggcttttgtttttagaatgaatttgaacattgaaaaggcaggcaatagggatgattctgtgaattctgctaaaactgagtagaaagaatgagtgtagagatgtcgacattgatcaactttctatcttcataagagatctgattctaacatatccatttagactcaagtagaatattgtgtatagagtgagtggcagtgagtaatttggtaaaaatttgctgacctgcttttattctttcctcctttctttcttcctttccttccttccttccttccgtcctttcctttcctttccctcccttccttccttctttccttctttctttcctttctttcctttcttcctttctttccttcctcccttccttttcttttctttctttcctttccttttctttcctttctttcctttcctttctttcttgacagagtcttgctctgtcactcaggctggagtgcagtggcgtgatctcggctcactgcaacctctgtctcccaggttcaagcaattttcctgcctcagcctcccgagtagctgagattacaggcgccagccaccacacccagctactgacctgcttttaaacagctgggagatatggtgcctcagaccaacccaaccccatgttatatgtcaaccctgacatattggcaggcaacatgaatccagacttctaggctgtcttgcgggctcttttttgccagtcatttctgatctctctgacatgagctgtttcatttatgctttggctgcccagcaagtatgatttgtcctttcacaattggtggcgatggttttctccttccatttatctttctaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctaggtaatatttcatctgctgtattggaacaaacactttgattttactctgaatcctacataaagatattctggttaaccaacttttagatgtactagtctatcatggacacttttgttatacttaattaagcccactttagaaaaatagctcaagtgttaatcaaggtttacttgaaaattattgaaactgttaatccatctatattttaattaatggtttaactaatgattttgaggatgagggagtcttggtgtactctaaatgtattatttcaggccaggcatagtggctcacgcctgtaatcccagtactccaggaggccgaggcaggtggatcagctgaggtcaggagttcaagacctgtctggccaacatggtgaaaccctgtctctactaaaaatacaaaaaaattaactgggtgtgctagtgcatgcccgtaatcctagctactctggaggctgaggcagcagaatcacttgaacccgggaggcggaggttgcggtgagccaagatcacaccactgcactccagtctgggtgacagagcaagactccatctcaaaaaatatatatatatatatatacacacatatattttatttcaactgttagacaagagtccaaaggccaaagaataaagttttaggccagtcctttattagaaaatgagtcaaatcccaaagcaagtttttttatgagttaatgaatataaatgactacatattttatgccttaaaaatcacttttaatgaatggtgttttatggcttgtaaatcagagttttaatcagtaaagaaagtttttaatcctcaaaaacacgttatcataaaagacactgtttggcatcaaatgtggtatttggccatgttcattagggtcattttaggaatctcatacattctacttagctatgcttaattcctgataccatggcattttctgaaatgtttcaaggatgacatctctgctgtttttaatttggtaatgatatctgctgatttattaagtgaaaaaagtaatggtgtcattaccttggatgaagaaacaaaaataaagcatttgccacatttttcaactttgttttcctttcttacaaaattgctataagctcattgcccccaaattggacaatatagggaataaaaaagataatttggggtggggttagacacgggtcttgttatgttgccgaggctggtctctaactcctggcctcatgcaatcttcctaccttggcctcccaaagtgctgggattataggtgtgagccacttcaccaagctgagatgccacctcttaaaagagagaataaggacagattacagccactgctcatgcctgtaatgtcagtactttgggaggccaaggtgggagaattgctcgaggccaagagttcaagaccagcctgggcaatgtagcgagacctgatctctatgaaaaggggggtgggggggaaaactagctggggccaggcgtggtgggtggcttacgcctgtaatcccagcactttgggaggccgaggcgggcagatcacctgagggcaggagttcaggaccaacctgaccaatatggagaaaccctgtctctactaaaaatacaaaattagccaggcttggtggcttatgcctgtagtcccagctactcgggaggctgaggcaggagaatcgcttgaacctgggaggcagaggtttcagtgagctgagatcgcgccattgcactctagcctgggcaacaagaatgaaactccatctcaaaaaaaaaaaaaatcagctggaaggtggcaaacacctgtggtcccagctactcaggaggctgagacaggaagatcacttgagtccaggaggtcaaggctgcaggtgagccatgtttgtgccactgcactgcagcctggatgacagaccgagacccttctcaaaaaaaaaatttttcccggtatttttttttgggggggggtttaattcttgttgcccaggctggggtgaattggggaattttgggttaagggaaccttcggcttcctgggttggggggtttttcctgtttaggcttccccagtagctgggattacaggcatgcaccaccacgcccggctaattttttgtatttttagtagagacagggtttctccatgttggtcagactggtctcgacctcttgacctcaggtgatccgcccaccttggcctcccaaagtgttgggattacaggcctgagccaccgcacccggcctgtactcttattctttaataataaaatatttctgtgtttctttagtcattttacataaacttttatttatttatttatttttatttatttatttttttgagacggagtctcgttctgttgcccaggctggaatgcaatggctcaatctcagctcactgcaagctctgcctcccgggtacacgccattcccctgcctcagcctccctagtagccgggactacaggcgcccgccaccacgcccagctaatttttttttttgtattttcagtagagacagggtttcactgtgttagccaggatggtcttgatctcctgacctcgtgatccacccgtctcggcctcccaaagtgctgggattacaggtgtgagccaccgtgctcggcccataaacttttatttttaaaataatgtcatgataaataatattgcttaggtgtctttaatatattagtaacatttctgttttattgtacatcaacatttatattcaaattaatgggtgaagagtactccattggactaggtatatcgtaatttaatctcctattattggacaactacattgtttctaaaattatactattcctatgactaaacctttgcatatatcttttatctccctaggatatatttctaaaactagcattgttgactgaaagtgtaaatacgtgttaaggtgtttgctacataatgccatatttcctttttaggaaactaagctactttggatttccaccaacactgtattcatgtacccatttttctcttaacctaactttattggtctttttaattcttaacagagaccagaactttgtaattcaacattcatcgttgtgtaaattaaacttctcccattcctttcagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttgtgagtgtatccatatgtatctccctaatgactaagacttaacaacattctggaaagagttttatgtaggtattgtcaattaataacctagaggaagaaatctagaaaacaatcacagttctgtgtaatttaatttcgattactaatttctgaaaatttagatctagataaagctatagtgtggattattttatgtatatttacttgagaaaataattattaaatattagtggaaaagctatactttgggtatgatataggactttcgaattggaattttcctttctatctgtaaaagcaagtaggtatagttttattccccagaaggcatctttttctcccccttgtctcacatgggtgaatttaccagcatatttaactaaattcagactggttccaaatgtactgccagatagtagcatttctctagtgtttgttttcatcctggcttgtaagaatgccctgccacttctgccctgcaatatcccttgctattaggattttggcatcaccttgggtccttaatgccagaaatgggaattgcttcatactgtggaaaaatacccattaaaatattaagaccagtaaaacctcgtttctgcttgggctatttgtggatttcagacatcctgagaagtttaccacccctgtaattaattgtcattgtcatcacttcataataaaaataattgcatggccgggcatggtggctcaagcctgtaatcccagcactttgggaggctgaggtggtcagatcacctaaggtcaggagatcaagaccagcctgaccaacatgaagaaaccccatctttactaaaaatacacaattagccgggcgtggtggcgcatgcctataatcccagctactcaggaggctgaggcaggagaattgcttgaacccgggaggcggaggttgcggtgagccgagattgcaccattgcactccagcctgggcaacaagagcgaaactctgtctcaataataagaagaagaattgcgtgaatatttctttaaaactatgatgagataacataccagattatcaaatggattcagtagtgggtgtgccatttattgcacactgagagatgaccaagtcattctgaaatatctttattaatatatccttcctaggatttttcatcctaacttctccataggtagttacttagcataacatctctgtggccagatgtatcccactactaaaagggcaaagtaagctgtggctgccctggtagatacaatgagtaagtgcacagtgatggctataaatgttttcatctcataatcccatgtccagaccagcaatttgctctgaaagctcttacctgtgtctgtttcaatggctcttgatcacttgcctgcacgtccagaattccttatttattcattgaaaattagcgttctttatccctttgttttgcaagttcagctttttagagatggctaaaatggtctaatctttcttggcaaaggcaattctgagctgcagattagactacaagtggcttgggtacatgttgtctttaaacaagcgaagaggaaaactttgagctctattcagacttggtgaagtgtggtaaatttatgatgaaagctactgactgtattacacatgattaattctgaagcccatattaagatgatcttttcagcagttcagcattgctcttctaactgaacagtttcaaggctgggatttcagcaattaatcagttcagaattgctaatgatctggcggagggtggtagcaaaagggggaggatgtcattagcttctctagcctgccttttttcagtgccctgtggcagtatggagtgaggcaacatgaaagaaagatggcctgaccttcatggcagtattgtgcaacacgtaaatactggtgtgagtggctgtggctatggctagtaaatgatggcccttggtaaacaaagttatttatcagacaatacctaccagctaggtcaactgtgcccataattgatctggttaatttcttttgctgcctattgatttttatttggttgatagataatagctagaggactctaaatttctttggggaagaacatgaaccccttctaagccttcttacgagagaattgatcgcttttgcactgacctttagtaacatcctgatttcagtgttttgtaactatcagagggttgagtcttggttttaagccatgtatatctgtagcataactttctgtgtaggctagttacctctcagcttataaagtgtaggctgataaatttatagtacagtagagtgtcactatgcaaagaaacgatcttagggaatcgaatgatatctgctattaaagcaaaattaatatatattttttctttttacttttttttttttttaaagacatgaaatctcactgtattgcccaggctggtcttggtctcagactcttgagctcaagcagtcctcccacctcagcttcccaaagtgctgggattataggcatgagctgccgtgtctggcccagtatatattttttaagttttaagttttgtggtacgtagtaggtttataatattattttgaatccttagttgtaattttatgtctgctgatgtgtacataatttttattaaactatttatttgagacttcaggtatcttttttttttttttgagacggagtctcgcactctcgcccaggctagagtgcagtggcgccatctcggcttactgcaagctctgcttcctgggttcacgccattctcctgcctcagcctcctgagtagctgagactacaggtgcccgccaccacgcctggctaattttttgtatttttagtagagacagggtttcaccgtgttagccaggatggtctcgatctcctgaccttgtgatctgcccgcctcagcctcccaaagtgctgagattacaggcgtgagccaccgcgcccagccgagacttcaggtgtcttagaattttttaaatgtaccctttctgagaaaaacagagacttaaagctaggataactggtattctattttttttttttttttttttttttacctccagcctgggtgacagagcaagactctgtctaaaaaaaaaaaaaaaaaaattcactttaaatagttccaggacacgtgtagaacgtgcaggattgctacataggtaaacatatgccatggtggaataactagtattctgagctgtgtgctagaggtaactcatgataatggaatatttgatttaatttcagatgctcgtgtacaagtttgccagaaaacaccacatcactttaactaatctaattactgaagagactactcatgttgttatgaaaacaggtataccaagaacctttacagaataccttgcatctgctgcataaaaccacatgaggcgaggcacggtggcgcatgcctgtaatcgcagcactttgggaggccgaggcgggcagatcacgagattaggagatcgagaccatcctggccagcatggtgaaaccccgtctctactaaaaaataaaaaaattagctgggtgtggtcgcgtgcgcctgtagtcccagctactcgtgaggctgaggcaggagaatcacttgaaccggggagatggaggttgcagtgagccgagatcatgccactgcattccagcctggcgacagagcaaggctccgtctcaaaaaaaaaaaaaaaaaacgtgaaaaaataagaatatttgttgagcatagcatggatgatagtcttctaatagtcaatcaattactttatgaaagacaaataatagttttgctgcttccttacctccttttgttttgggttaagatttggagtgtgggccaggcacggtggctcacacctgtaatctcagcactttgggaggccgaggcgggtggatcacctgaggtcaggagttcgagaccagcctggccaacgtgttgaaaccccgtctctactaaaaatataaaaattaggtgggcgtggtggcaggcacctgtaatcccagctactcaggaggctgaggcagcagaatcgcttgaacccaggaggtggaggttgcagtgacccaagatcgcaccattgcactccagcctggggacaagagcgagattcttgtctcaaaaaaaaaaaaaaaaaaaaaaggtttggagggtggtgagctgagatagtcaactattaactcctatctacctgctgggactacactggtgaggtggagcctaagtcctaaaacaacaagtgaggcagctggacgcggtggctcgcatcagtaatcccagcactttgggagcctgaggcgggcagatcacaaggtcaggagttcgagaccagcctggccaatatggtaaaacccagtctctactaaaaatacataaattggctgggcgtggtggtgtgcacctgtaatcccagctacttgggaggctgacacagaagaattgcttgaactctggaggctgaggttgcagtcagctgagatcctgccactgcactccagcctggcgacagagtgagactctgtctcaacaacaacaaaagaaagaacaagtgaggcaaaacctggagaccccagcttcatgtaacacctagtttgagtattgttgagagtttttcaggaaaaaagtctgataacagctccgagatagtcttaacatatgaaaaagcaaaaaagggaggagacagatcatttgtcctatacctttctcttttaaggttttaattataacttgtgtaatacaggagacctctgggtgtttttagttgactataaactaaatctgagtacacatttcagggctgctaaaaatgcttatttgaaactgggccgtattaacacaagcagaggctctggagcaagtgaagtacagatccagagccccactgtattctccaatggagtgattgcctgaaagatgatgtcagttttaagcaccgtgcttggtttttaacatggtcactgacaaattggagagtgtttatccagaggtagatggtaaagatacataaaagtaacttgaaatactgtcttttgaagaagaaatgagaagatttaaggaaataagacactgtcttcaagtatctgaagaaccgttacccggaagagaactgttatctggaacaggattaagactcactcatggggctccagaaagcagacgagtgcatggaggacgcagaagatgcagattgtgtggctcaactctaaaatctttctaacaaaattagttctctggatgtgttccagttcacttgatgatgattcttttgtttttgtttttgtttttgaggtgtagtttttcactcttgttgcccaggctgctggagtgcaatggcacgatcttggcttgctgcaacctccccctcccgggttcaagcgattctcctgcctcagcctcccgagtagctgggattacaggaatgcaccaccatacctaattttgtatttttagtagagacagggtttttccatgtcagtcaggctggtcttggactcccgacctcaggtgatccacctacctcggcctcccaaagtgctgggattacaggtgtgagccatcgcgcctagcctatgatgattcttttcacagagatacaggcacttaaggagaggatctaaaccccttggacacattgccgttgaacttctaagatcttaggtttccacttactcatgaaaattataccacagggtcagagggtagtgttcattggagccaggtgccagaacaagttattacaaactactattttagagaaaaatgtcattaaagtttaagataccttaagctataggtttgcatcaaagttaatgaaaggtaaaaagatgccaagcgtggtggctcaggcctgtaatcccagcgctttggggggccaaggcgggcagatcacgaggtcaggagatcgagaccatcctggctaacacggtgaaaccccatctctagtaaaaatacaaaaaattagccgggcatggtggcgggcatctgtagtcccagctactcaggaggctgaggcaggagaatggcatgaacccaggaggcagagcttgccgtgagctgagatccagccactgcactccagcctggctgacagagcaagactgcatctcaaaaaaaaaaaaaaaaaaaaatgcaaatcaaatctaaagtagttcagtctttaaactcaaagccaatacatttgctttgaactacaaatgaactgaagtttttaagtgtaataaatgttactaaatcggcttttgtagcagttaaacaaaaaacttcaaaaattgtaaggattctgtgagggagcatggctgctgctgctgctgctgcttgcagatagcctgctgtgtttaggatttagttaaatacatttctcctgtttaaaactaaatggtctttccttagtttgcttagttcttcagaagggcctttgaaacactgggaaataaacaagtgattctttagctactgctttctgaaatacttatataaaagctctgcactgtattctcccatccctctcaggggaatattagagggttaggactccccaggtagacattctaggggtgaaaatttgtcattacattgacatttcagatttaggttttcaacaatactgttttcttctttcacatattgccatctagtaatatagatgttctccgtccacattaatcaaaactattgacatggataattcctaattccttgaacactataatggagatctatagctagccttggcgtctagaagatgggtgttgagaagagggagtggacagatatttcctctggtcttaacttcatatcagcctcccctagacttccaaatatccatacctgctggttataattagtggtgttttcagcctctgattctgtcaccaggggttttagaatcataaatccagattgatcttgggagtgtaaaaaactgaggctctttagcttcttaggacagcacttcctgattttgttttcaacttctaatcctttgagtgtttttcattctgcagatgctgagtttgtgtgtgaacggacactgaaatattttctaggaattgcgggaggaaaatgggtagttagctatttctgtaagtataatactatttctcccctcctccctttaacacctcagaattgcatttttacacctaacgtttaacacctaaggtttttgctgatgctgagtctgagttaccaaaaggtctttaattgtaatactaaactacttttatctttaatatcactttgttcagataagctggtgatgctgggaaaatgggtctcttttataactaataggacctaatctgctcctagcaatgttagcatatgagctagggatttatttaatagtcggcaggaatccatgtgcagcaggcaaacttataatgtttaaattaaacatcaactctgtctccagaaggaaactgctgctacaagccttattaaagggctgtggctttagagggaaggacctctcctctgtcattcttcctgtgctcttttgtgaatcgctgacctctctatctccgtgaaaagagcacgttcttctgctgtatgtaacctgtcttttctatgatctctttaggggtgacccagtctattaaagaaagaaaaatgctgaatgaggtaagtacttgatgttacaaactaaccagagatattcattcagtcatatagttaaaaatgtatttgcttccttccatcaatgcaccactttccttaacaatgcacaaattttccatgataatgaggatcatcaagaattatgcaggcctgcactgtggctcatacctataatcccagcgctttgggaggctgaggcgcttggatcacctgatgtcgggagttcaagaccagcctgaccaacatggagaaaccccgtttctactaaaaatacaaaattagccgggcttggtggcacttgcctgtaattccagctactcgggaggctgaggcaggagaatcacttgaacctgggaggcgggggttgcagtgagctgagatcgcatcattgcactctaacctgggcaacaagagcaaaactccatcaaaagaaaaaaaaaatcgggtgcagtggctcatgcctgtaatcctaacactgtgggaggccaagacaggcagattgcctgagctcaggagttcgagatcagcctgggcaacatggtgaaaccctgtctctactaaaatacaaaaaattactcagcgtggtggcatgcgcctttagttccagctactcaggaggctgaggcaggagaatctcttgaacccgggaggtggaggttgcaatgagccaagatcgtgccactgcactccaacctggcaacagagcgagactccgtcttaaaaaaaaaaaaaattttgcagcgcaaaccaggatatcctctgttctcatttgttctagatttcaaaagaaacagtcctttctttggggaaaagagaaaggaaaaggagttttataaaaggaaagaaaagattcataagaacaagaagtgggcccacttgcatatacctttgtagaaaactgttcactgttgttgaagaaaagctcttcatattaatatgcagtccagatgcagtggctcacacttataatctcagccctttgggaggctgagacaggaagattacttgaggccaggagtttgaaaccagcctgggcaacatagtgagactctgtctccacaaaatttttttttaattagccgggcatggcagtgtgcttctgtagtcttagctactgaggaagctaagccagaagaatcacttgagcccaggagttcaaggctgcagtgagctatgatcataccattgcactcttgcacttgcacagagcaagaccctgtctcttaaaaaaaaaaaagtgtgtgtgtgcatatgcatatatacatatatatacatgcaaatgtatctgtttataattcagattgcttcaaaaagatgttgcactttatgatactgagaacagtgagaagtaaataagatagagtgtaggaggaggaataatttcagaacagccatctgagaacttctgtgacaacagatcaggcaaaatgaaatgtgaaagtaattttataggccaggcgtggtggctcatgcctataatcccagcactttgagtggccaaggcaggtggatcacttgaggtcaggagttcgagaccagcctggtcaacatggtgaaaccttgtctctactaaaaacacaaaaaaattagtcgagcgtggtggcatgtgcctgtaatcctagctgctggggaggctgaggcaggagaatcacttgaacccgggaggcggaggttgcagtgagcctagattgcaccactgcactccagcctgtgagacagaatgagaccctgtcttaaaaaaaaaaaaaaagtaattttataaactattgtgcacaattcgatgtattcataattaattaaatgattatttttgttggttttaacttttattcagtggctatttattgggagcctactgtgttctgggcactaggaatgcaacagtaaataagactaactaagtccctggtaggattcaggttctgtcgaggggagatacacaataaagatgaatttaagataacaataaatgctatggagaaatatacagaacagtggaatagtattagctgtcaaaggttgttgattactttcgtttaaggaggccagggaaagcctttctgaaaaaattgagctgagacctaaataacaagaaataattgtccttgaaaaatgaagggaatgcatcttataggcagaggaatagcaaacataaaggtcttgaggtaataatgagtgtggttttttgatttctgtattttggtttttttgagatggtgtctccctctatcccccaggctggagtgcagtggcacaatcttggctcactgcaaactctgtctcctgggttcaagcaattctcctgccttggcctcctgagtagctggtattacaggcacgcgtgctaccacacccgactagtttttatttttagtagagatggggttttaccacgttggtcaggctggtctcaaactcctgaactcaagtgatccaaccacctcaacctcccaaagtgctgggatcacaggcgtgagccaccatgcccggccagagcttggtttattttttaaaagataggccaatgttggtcgtgtgtggtggctcgtgcctataatcccagcactttgggaagccaaggcaggcaaatcacttgaggtcaggagttcgagaccagcctggccaacatggtgaaaccccatctctactaaaaatacaaaaaactagcatggtgtggtggtgtgtgcctgtaatcccagtgcctgtaatcccagctactccagaggctgaggcaggagaatcacttgaaccgaaaggtaggagttacagtgagccaagatcgcatcactgcactccagcctgaacgacagagcaagactcctgtctcaagaaataataatgataaaaggttcgggcacagtggctcacacctgtaattccagcactctaggaggccgaggcaggcagatcccctgaggtcaggagtttgagaccagcctggccaacgtggcaaaaccccatctctactaaaaaatgcaaaaattagctgggcacggctgggtgtggtggctcattcctgtaatcccagcactttgggaggtcaaggcggacagatcactgaggtagaaaccctgtctctactaaaaatacaaaaatttgcccagcgtggtggcgcgtgcctctaatcccagctacacgggaggctgagacaagagaatcacttcatcaacccgggaggtggaggttgtggtgagctgagatcgcaccattgcactccagcctgggcaacaagagtgaaactccatctcaaaaacaaaaaaaaattagctgggaatggtggcatgtgcctgtaatcacagctacttgggaggctggggcaggagaatcgcttgaacccaggaggcggagattgcagtgagctgagattgcgccactgcactccaggctgggcgaaagagcaagactccgtctcaaaaataataataataataataataggccagtgtagctggagtaatttgcaaattatgtgtggaggcagagattacacaaggaatgggagaaggtcatagatgagggccagatcacatagtatttggtggtaaggaattcagattttatccttgtggtaattggtggtgtggagatggttaaaaacaaggttggtttgggatgggtttgaagagaggacttgctaatggattaaatttggaggataaggtaaagagaaattgaaggagtgacacttgggttttggcttgaacaatagatcttgttagtaatattaaattagatgaagaaggcatggtagggaatatgggggagtgggaaaggcaggaagcaggaatggaaccaggaactctgttttagatgtgagaatttgttgttgttgttgttgttgttgttgttgttgttgttgttgttgtgacagcatctcgttctgttgcccaggctagagtgcatggagtgcggtagcacgatctcagctcactccaacctccgcctcccggttcaagtgattttcctgcctcagcctcccgagtagctgggattacaggcacctgccacaatgcctggctaatacttgtatttttagtagagatggggttttaccatgttggccaggctggtcttaaactcctgacctcaggtaatccacccacctcggcctcccaaagtgctgggattgcaggtgtgagccactgtgcccggccagatgcatgaattttgagatgtatactagacttctggatagagaagttaagtaggcagttggacacattgtatgaagctcaggggtacaaggaggactatgaacatgggagtcttctgacaaatttatcactagactcctcattcaagtaactaggaaatgtcagatattcttcccctagtaatagccagtggttatactcttgcctttagttttcttcacaatactcttggcaacacataaggccttccctacaatctgagtttcagtcagaattgtttctgagcgttcttcctcaaatttctccccagtctcattattctttattctcatgtccatgaccagtcataatagtaattatgaaaaacctctaactttctttagtgcattgaatgtatattttatcattttggttgtgttaactgtaaatctctcagtggaaatctgaaaagcctttatttccttagatgataatatacaattgatttaggagatagggaatttttcagttacctttataacagcacagtattagcagtctaatctaaatgctaagtgaatgttttgagaggagatagatgttgaaaattaaaatacattaagtcccagtgaggtgaaaagccgattgttaagttctgcacacaaaagatttgcttcagtgaattgatttcaacagctgagatcctagtcatttcacctggtctaccaaaaagaatgattttacttgcttttggtcaaatctctgcccagcaattctttttctttctttcttttttttgttttatgtgtgtgtgtgtgtgtgtttttttttagcagagtctcactttgtcacccaggcgggagtgtggtggtatgatcacagttcactgcagcctccaactcctgggctcaagtgatcctccagcttcagcttttcaagaaattgggactgcaggcacatgcaactatgcctggctgaggttttatgtatcttttttctagagaaggggtctcactgtgttgcccagctgggtctccagctcctggtctcaagctgtcctcctgcctcagcctcccaaagtgccaaagtgctagggttataggtgtgagccattggtgcccagctactgcctgcctggcaattctgaatgccttaaattttttttttttttttttttttttttgagacagagtttcactctgtcacccaggctggagtgcagtggcatgatcgtggctcacagcaacctctgcctcctggattccagcaattctcatgcctcagcttcccgagtagctgggactacaggtgcatgccaccacgcccagctaatttttggtttttttgtttgtttgtttgtttgttttgagacggagtctcgctcagttgcccaggctggagtgcagtggcgtgatctccgctcactgcaagctccgcctcccgggttcacgccattctcctgcctcagcctcccgagtagctgggactacaggcgcctgccactacacccggctaatttttttgtattttaagtagagacggggtttcaccgtgttagccaggatggtctcgatctcctgacctcgtgatccgcctgtctcggcctcccaaagtcctgggattacaggcgtgagccaccacacccggcctaatttttttttttttaattttatttttaattttttgagatgcgagatggagtctcgctctgttacccaggctggagtgcagtggcaccatctcagctcactgcaacctccacctcctgcattcaaaagattctcctgcctcagcctcccaagtagctgggattacaggtgcctgccaccacgcccaactaattttttgtatttttagtagagatgaggtttcaccatgttggtcagactggtgtcgaactcctgacctcaagtgatctgcctgcctcagtctcccaaagtgctaggattacaggggtgagccactgcgcctggcctgaatgccttaaatatgacgtgtctgctccacttccattgaaggaagcttctctttctcttatcctgatgggttgtgtttggtttctttcagcatgattttgaagtcagaggagatgtggtcaatggaagaaaccaccaaggtccaaagcgagcaagagaatcccaggacagaaaggtaaagctccctccctcaagttgacaaaaatctcaccccaccactctgtattccactcccctttgcagagatgggccgcttcattttgtaagacttattacatacatacacagtgctagatactttcacacaggttcttttttcactcttccatcccaaccacataaataagtattgtctctactttatgaatgataaaactaagagatttagagaggctgtgtaatttggattcccgtctcgggttcagatcttagctgataagtggaagagctgggactttaagcagatgagaatctaaagactttgctcttttcacttcactggggtgtctttctctctctctctcttgctctctctctctctttttttttttcccaagacggagtctcactccattgcccaggccagagtgcagtggtgcgatctcagctcactgaaaactcatcttgcccaggctggtcttgaacccctgaccttgtgatcctcccgccttggcctccccaagtgctgggataggcgtgagccaccgtgcccagccaataatagctaaaatttatataatgttcactgggccaggcacagcggctcgttcctgttatcccagcactttgggaagctgaggcaggcagatcgcttgagccaaggagttcgataccagcctgggcaacatggcaaaaccccatctctaccaaaaaaaatatacaaaaattagccaggcgtggtggcatgtacttgtagttccagctactcggaaggctgagttgagagtatctcttgagcccaagaagaggggactacagtgaacggagattgcgccactgcactccagcctagacgacagacagaagatctcaaaagaaaaaaaaaaaaaaaagatcactttatgctgggactgctctaaaggcccaaccatgttttaactaattaacaattttatgacaactctatgagctatgtactgtaattatgcctatattacagatgtgaaaattgaggctcagagaggttgaataagttgctcaaagtcacacaggtaataagtgatggaactagaagttgaactcaggaagtctagctccaagtctaaattctttgttaatttatttttcgggccagagtcttactctgtcacccaggctggagtgcagtgccactatctctgctcactgcaaccttcacctcccaagttcaaaccttgttcaattcttgtgccttggcctcccaagtggctaggattacaggcatgtgccacaacaactagctaattttttgtctgattctgttggccagtctggagtgcagtggcgcaatctcagctcactgcagtctccagctcccaggttcaagtgattctcgtgccttagcctcccaaatagctgggattacaggcacgtgccaccacaccgagatagttttttgtatttttaatagaaacaaggtttcaacatgttggccaggctggtctcaaattccagacctcagatcatctgcccgcctcaggctcccaaagtgctgggattacaggcatgagccactgcacccggccttaatttttatatttttattagagatggggttttgccatgttggccaggctggccttgatctcctggcctccagtgatccacccgccttggcttcccaaagtgctgggattacaagcatgagccactgcacccggcctccaattctaaactcttaacaacaatactatagtttcttgaaaagttgttgaaggcttcacggagggaaaaaaaatggagcattctaacaactttgcagatgagacccaagaagactcaatgactttctcctgatcatattgtagcagatgacttagccagaactctgacttcctcacagggagaaagtctgcaagatttcacacttacctgtcaggcctgagctggctgctttctcagctccctaagtgctatgttcccagtctgcttttcttcctttttcaagtgtgcactaccaggcatttcagaacatcccaggctggtcgcggtggctcacacctgtgatcccagcactttgggagcccaaggcgggtggatcacctgaggtcaggagttcgagaccagcctggccaacatggtgaaaccccatctctactaaaaatacaaaagttaactgggcgtggtggtaggcacctgtaatcctagctcaggattactcgggaggctgaggctagagaatcggttgaacccaggaggcggaggttgcagtgagccaagattgcgccactgcactctagcctggggacaagagggagacttcatctcaaaaaaaaaaaaaaaatcccagctgggcacagcggctcacttctgtaatcccagcactttaggaggccaaggcaggaggatcacttgagcccaggagttcaagactagcctgggcaacatagtaagaccctgtctctacaaaaaaatttaaaaattaattgggtgtcgtagcacactcttgtattcccagctactcaggaggctgaggtgagaagaatgcttgagtctgggaggtcgaggctgcagtgagccatgatggtgctactgcactccagcctggccaacattgtgagaccttgtctcaaaacaaaacaaaacatccttctactgagcactttctgtccctttatagaaacttaagagggaaccagtagaggtaatttcctaaggaaaactgctttgggacatgatcacaaatgaagcctggagttttgaactgctgaggtcagcctgtttttaccttctgagcctatcaagtaattgttccagatgccaagaaaagctgctggccttatttctgcttctgcctttaccacaggggagcgccatgtgagccagtcctctgtttttcctccactgtatgctaggcagtattagcaccagattcttcccctctttaaaaagaaattctagtgctttggattttttcctccatgcagaatagcaatgatggaaagtatgtggtcaaagtaatgacattctgaaaatactaaatgtcaccatagtatttttctctggaagagaaatgtatatgtagaggtgaaacttcaaatttctttttttttttttttaagacgaagctttgctcttcttgcccaggctgaagtacaatggcgtgatcttggctcaccgcaatctctgcctccagggttcaagtgattctcctgcctcagcctcctaagtagctaggattacaggcatgtgccaccacgcccagctgattttgtatttttagtagagatggggtttctccatgttggtcacgctggtcttgaactcccgaccccaagtgatccacccacctcggcctcccaaagtgctaggattacaggccaccgcgcccggcctgaaacttcaaatttctttttttttttgagacagagtctcgctatgtcacccaggctggagtgcagtggcgccgtctcggctcactaccagctccactccacctcctgggttcacaccattctcctgcctcagcctcccaagtagctgggactacaggtgcccgccaccatgcccagctaattttttgtatttttagtagagacgggttttcactgtgttagacgggatggtctccatctcttgacctcgtgatccgcctgcctcagcctcccaaagtgctgggattacaggcgtgagccactacgccaagcccgaaacttcaaatttcttatctcataactaggcatccttatcactgagtgttagcctggatataaacattcctaatcttttgtacttttcatgtcagcatttggctccacttggctgcctggggagaacttctagcattatgagcatgcaggtcctatcaacaggttgggggtgcggtttattcatacaggtagtgagagtggcacagatggatgctgtcccttaaaacaaacagacttgtctttgggagcctgaggcgggtggatcatgaggtcaggagttcaagaccagcctggccaacatagtgaaaccccgtttctactaaaaatacaaaaaattagccgggtgtggtggtgtgcacctgtaatcccagctactagggaggctgaggcaggagaatcacttgaacccaggaggtggaggttgcagtgagccgagatggcaccattgcactccagcccaggcgacagtgcaagactgcgtctcaaaaaaaaaaaaaaaacacacagacttgtcctactgccatttcttttcactctggcggtaaagtaagagtgtgtgtgtgtgtgtgtgtgtgtgtgtgtgtgtgtgtgtgtgtgtgtgtttctgtccgtctgtctgtcaagggggagggtgaccactttctaaaaggccatccgtgtatttttagcttcctgatttttttctctatcgcagtctctttgaagccaggtgaattttaggccttggcaattttctttttattgcaatgggaaggtcaagacactgagagtcacccaaaacatatccatccaaaatgatacaattttagggtttatttttaagtgatacccaagttatttgctaagaacctatgccagtgtgtttatgagaatttgcactgtcccacactgttgccaccagccacatgggactgtttaaatttaaattttacaaattagccagtcatggtggtgtgcacttgtagtcccagctacttaggaggctgaggcaagaggattgcttgagcccagaagttcaatactgcagcaagctatgatcgtgccactgtactccagcctgagtgacagaatgagacctcatctcttcaaaaaaaaagaaaaaaattaaaatatgaagtttagttcttcattcaccctaaccacatctccagtgctcaataactatatgtgactcatggctaccttattagcatagatatagaacattgtgactatcacagaaagttgttttgaacagtgttgccaagccctgtaagtggaagaggcagtgcagtgtgatctgtgtcttcaggaaaccaggtagtcagactagttcaatgaggagaggcagaacctggcttcacttctagattaaaaactgcttaggtggcctaaagatacaatggccattctcagagtagtgagaaggaaggaacagatgtttagggggctagaagaaagtcagagagggccgggcgcagtggcttatgcctgtaatcccagcactttggaaggccaagacaggcagatcacgaggtcaggagttcgagaccagcctgaccaacatggtgaaaccctgtctctactaaaaatacaaaaattagccgggcgtggtggtgcgcgcctgtaatcccagctactcaggaggctaaggcaggagaatcgcttgaaccctggaggcagaggttgcagtgagcccagatcgcaccactacgctccagcctaggtgacagagagagactccgtctcaaaaaaaaaaaaaaaagtcagaggagacaaggagcatgtacacctaaaatcaacatagacccctctgttgatggggtcatagtgagtacttgaggtaccaagtctggataaacatcaaacttcagccaataactttgagtttctagccatccaagcctcttattaaacatacagaaggaccttttttcccttgcatctaacaagttaaagcacctgcagagatcattagggaggagccttggcctgattggtgacaaaagtgagatgctcagtccttgaatgacaaagaatgcctgtagagtgcaggtcaactacatatgcacttcaagaagatcttctgaaatccagtagtgttctggacattggactgcttgtccctgggaagtagcagcagaaatcatcaggtggtgaacagaagaaaaagaaaagctcttcctttttgaaagtctgttttttgaataaaagccaatattcttttataactagattttccttctctccattcccctgtccctctctcttcctctcttcttccagatcttcagggggctagaaatctgttgctatgggcccttcaccaacatgcccacaggtaagagcctgggagaaccccagagttccagcaccagcctttgtcttacatagtggagtattataagcaagatcccacgatgggggttcctcagattgctgaaatgttctagaggctattctatttctctaccactctccaaacaaaacagcacctaaatgttatcctatggcaaaaaaaaactataccttgtcccccttctcaagagcatgaaggtggttaatagttaggattcagtatgttatgtgttcagatggcgttgagctgctgttagtgccaacatgttagtgagaaaatatctttggataggtaaaaatcaaggaggagttctcctcttcctaaaccatcttaatttacttacatagaagaaagcacagcagctggcccaccacggacgggcccagagcaggggaagattctcggtgaacatttctttttttttttctttttttttgaggtcgagtctctgttgcccaggccagagtgcaatggcgcgatctcggctcactgcaacctccacctcccgggttcaagtgattctcctgcctcagcctcccaaatagctgagactacaggcgtgtgccaccacgcccgactaattttttgtattttttttttagtagagacggggtttcaccgtgttagccaatatggtcccgatctcctgacctcgtgatccacccgcctcagcctcccaaagtgctaggattacaggcatgagccactgtgcccagccctctccatgaacattttctaattaaacttgacacttaatacaatgttatgcttaggactgctataaagcttacctctggagttgcgcagcacaaaggccttggtgtgtgtataaatttggtttgttcttttcacagcaaaagctacccacctttgcctcctgtgcctgcttctgcccagggacttaggtcctcttacaccttagagaaaggccttagcatctggtcacaggcagatgagtgacagcaagaaaacctggctgcaatgtaattttgtttccatcctctttattagttatcaattggatttttatgaaatttccaagttccactcaaggatttctcagtgtttttttactttggtatagtggaaaccagggttgccagaaagtattattttgggggtgagttagtcaaccttcgttcagtcagacagacaggagcacctcagcaattcccagaaacgggctgatgggaaagagcaacatacatgaatgtcttgaagaacacagccaacagagcccattgggcagttctgattttccaggtacacagcatctccacagtctcttctgatttttattcccctgagtatatggattccagctcagcatgtagcctttccctgctgagtctctaaccaggataacatgtatttttttgactggatgaattatcttcccatctcttgacatttacagtaattaccaccaagtatggtattttcagtggccgtgattatcagttaccaacacagaattaggatgaagggaggaagggagggaaggaaggtgggtgttttttcacacagtgtcttagccagcaatttagcaaattaatggaaattagatctttgatttttttttctttcaagcattttatttgagagactatcaaaccttataccaagtggccttatggagactgataaccagagtacatggcatatcagtggcaaattgacttaaaatccatacccctactattttaagaccattgtcctttggagcagagagacagactctcccattgagaggtcttgctataagccttcatccggagagtgtagggtagagggcctgggttaagtatgcagattactgcagtgattttacatctaaatgtccattttagatcaactggaatggatggtacagctgtgtggtgcttctgtggtgaaggagctttcatcattcacccttggcacagtaagtattgggtgccctgtcagagagggaggacacaatattctctcctgtgagcaagactggcacctgtcagtccctatggatgcccctactgtagcctcagaagtcttctctgcccacatacctgtgccaaaagactccatctgtaagggatgggtaaggatttgagaactgcacatattaaatatactgagggaagactttttccctctaactctttttcccatatgtccctccccctcctctctgtgactgccccagcatactgtgtttcaacaaatcatcaagaaatgatgggctggaggctgggcatggtggctcatgtctgtaatcccagcactttgggaggccgaggcaggtggatcacttgtcaggagtttgagaccagcctggccaacatggtgaaaccccatctgtactaaaaaaaaaaaaacaaaaagtagccaggcctggtggagcatgcctgtaatgccagctatttgggaagttgaggtgtgagcatcgcttgaacgtgggaggcagaggttgcagtgagccaagattgcaccactgcactccagactgggtgacagagtgagactttgtctaaaaaaaaaaaaaaagagagagagagaaaagctaggtgcggtggctcacgcctgtaatcccagcactttgggaggctgaggtgggcagatcacgaggtcaagagatcgagaccatcctggccaaccaacatggcgaaaccccgtctctactaaaaatacaaaaattagctgggcgtaggggcgcacgcctgtagtcccagctacttgaggggctggggcaggagaatcgcttgaaccccggaggcggaggttgcagttagccaagatcgcgccactgcactccagcccgggcgacagagcgagactccgtctcaaaaaaaaagagagagagagaaatgatgggctgggccagtgccccacccctgtaatcacaacactgggaggccaaggtgggagaatcgcttgagcctgggagctgaagaccagcctgggcaatacagtaggacctcatgtctacaaaaaaattattaaaaattagccaaggctgggtgcggtggctcatgcctataatcccgggggtgaagttgagcccaggagtttgagaccagcctgggcaacatggcaaaaccctgtctctaccaaaaatacaaaaaaattagccaggggtggtggtacgtgtctgtagttccagctacttaggaggctgagatggaaggattgcttgagcccaggaggcagaggtggcagtgagctgagatcacaccactgcactccagcctgggtgacagagcaagaccctgtctcaaaaacaaacaaaaaaaatgatgaagtgacagttccagtagtcctactttgacactttgaatgctctttccttcctggggatccagggtgtccacccaattgtggttgtgcagccagatgcctggacagaggacaatggcttccatggtaaggtgcctgcatgtacctgtgctatatggggtccttttgcatgggtttggtttatcactcattacctggtgcttgagtagcacagttcttggcacattttaaatatttgttgaatgaatggctaaaatgtctttttgatgtttttattgttatttgttttatattgtaaaagtaatacatgaactgtttccatggggtgggagtaagatatgaatgttcatcacaaaaacataaatcaaggccgggcatggtggctcatgcctataattccagcactttgggaggtcaagatggaggtcaaggtgggagcctagaagttcgagaccagcctgggcaacataaggagacttcatctgtacaacaaatttaaaaagtagctgggtgtggtggcagatgcctgtagtcgcagctacttgggaagctgaggtgggaggatcacttgagctcaggaggttgatgcttcagtgagccacgatcacaccactgtactccagcctgggcgacagagcgagaccgtgtctcaaaaagaaaaaagaaagtataaatttacacaaaaacaataaaataatcccagtaattccaccacttggagatgatcaccataaaactccaccaggcatatgtgcgtatatatacacgtgtattttataaaatgtgatcataattacactgttttgcttttttccttaagatattacatacatttttccacatcgttaaattacagtgctgttttcctggtggctttcctttaacagattgaagttcatgttaatacagttgccagaggctgtgggctttcactgtcaccaggagtcactcctagggcctcttcagagcaaggccttatgtcctgaagcattgccttttttttttttttttgaggtggagtctcactctgtcacttagcaggctggagtgcagtggcccagtcttggctcactgcaacctccgcctcctgggtttaaatgattctcctgcctcagcctcagggcggatcacctgacatcaggagtttgagaccagcctggccaatatggcgaaaccccatctctactaaaaatactaaaaaaaattagccaggcatggtggcacgcacttgtagtcccagctacttgggagactgaggcaggagaatcgcttgaacccaggatgttgaggttgcagtgagctgagatcacaccatcacaatccagcctgagtgacagagtgagactccatctgaaaaaaaagaaaaaacaattagcctggcatggtggcaggcacctgtaatccctgctacttgggaggctgaggcaggagaattgcttgaacccgggaggtggaggttgcagtgagctgagatcgtgccattgcattccaggctgagcaacaagagcaagactccgtctcaaaaaaaaaaaaaaaaaaaaaaaaaggccaggtgcagtggctcacgcctgtaatcccagcactttgggaggccaaggtgggtggatcacctgaggtcaggagttccagagcagcctggccaacattgtgaaacccccgtctctactaaaaatacaaaaattagctgggtgtgatggcatgtgcctgtaattccagctactcaggaggcagagacaggagaattgcttgaacccaggaggcggaggttgaatgagccgagattgcgccatcacactctagcctcggcgacagagcaagactccgtctcaaaaaaaaaaaaaaaaaaattagcttctacctcattaatcctaagaactcatacaaccaggaccctggagtcgattgattagagcctagtccaggagaatgaattgacactaatctctgcttgtgttctctgtctccagcaattgggcagatgtgtgaggcacctgtggtgacccgagagtgggtgttggacagtgtagcactctaccagtgccaggagctggacacctacctgataccccagatcccccacagccactactgactgcagccagccacaggtacagagccacaggaccccaagaatgagcttacaaagtggcctttccaggccctgggagctcctctcactcttcagtccttctactgtcctggctactaaatattttatgtacatcagcctgaaaaggacttctggctatgcaagggtcccttaaagattttctgcttgaagtctcccttggaaatctgccatgagcacaaaattatggtaatttttcacctgagaagattttaaaaccatttaaacgccaccaattgagcaagatgctgattcattatttatcagccctattctttctattcaggctgttgttggcttagggctggaagcacagagtggcttggcctcaagagaatagctggtttccctaagtttacttctctaaaaccctgtgttcacaaaggcagagagtcagacccttcaatggaaggagagtgcttgggatcgattatgtgacttaaagtcagaatagtccttgggcagttctcaaatgttggagtggaacattggggaggaaattctgaggcaggtattagaaatgaaaaggaaacttgaaacctgggcatggtggctcacgcctgtaatcccagcactttgggaggccaaggtgggcagatcactggaggtcaggagttcgaaaccagcctggccaacatggtgaaaccccatctctactaaaaatacagaaattagccggtcatggtggtggacacctgtaatcccagctactcaggtggctaaggcaggagaatcacttcagcccgggaggtggaggttgcagtgagccaagatcataccacggcactccagcctgggtgacagtgagactgtggctcaaaaaaaaaaaaaaaaaaggaaaatgaaactaggaaaggtttcttaaagtctgagatatatttgctagatttctaaagaatgtgttctaaaacagcagaagattttcaagaaccggtttccaaagacagtcttctaattcctcattagtaataagtaaaatgtttattgttgtagctctggtatataatccattcctcttaaaatataagacctctggcatgaatatttcatatctataaaatgacagatcccaccaggaaggaagctgttgctttctttgaggtgatttttttcctttgctccctgttgctgaaaccatacagcttcataaataattttgcttgctgaaggaagaaaaagtgtttttcataaacccattatccaggactgtttatagctgttggaaggactaggtcttccctagcccccccagtgtgcaagggcagtgaagacttgattgtacaaaatacgttttgtaaatgttgtgctgttaacactgcaaataaacttggtagcaaacacttccaccatgaatgactgttcttgagacttaggccagccgactttctcagagccttttcactgtgcttcagtctcccactctgtaaaatgggggtaatgatagtatctacctcctaggatttattgaggcagcttaaataccttttgtatttcctgttgctgccaaaacaaattgttgcaaggtcagaagtctgaggtggctcaactgtttctttgtttcaggtttcatgaggccaaaataaaggtgttcgcagggcgtgttcccttctagaggctctgggtccttgcagttctaggactaagatccctgtttcccactggctgttggctgggcatcattctcagcttcttgaggctccccacattcctaggctcctggcctgtctgcctccatcttcaaaaccagcaatgggtggtcaagtttttctcacactgaatcttgctgactactgtatctttctaactcctgccagagacatttctctgtttctaagggctcaagtgattagattgcacccacttggtaatccaaagtgatcttcatatcttaaggcccatagccttaattatagctgcaaagtcccttcgcagcagtacctagattactgttggaatgaataaccagaagacagcaatcaagggaggacatctttagaattctgcctaccacttgtatttaacatgcttaatccacagatgacactctctaccattatttcctggtcctcacactgctcagagattggaatcctttttaagcaaagagaatgaagtcatcacatagttcagtcctgctgtatttgctggaaacagtgaaggaagatagagaaaatggagctaactgccaatattaccattttataatcagtcctcaatcatagccctatgaagtgggtatttgttacctcattggaaaaatgggagttgaatctcaagttccttgtttgtaagattttactcagatttgcacagctaaaaatgactacatggagacccaaagccacctttctgttcccatcatcagctttccatctgcctctgtcactgaccccgggacagaaggttcaagccttaagggaatttggagagagaactagattttgaggggaactcacactcacttcccttttgggccacagtaggagacagtaaaagcagccccatgtcaggcaaagggtcttacaggagtggatcatggctgctgtttccacttctctctggcttcccagcttatgactgtgtatcttagttgtcaaagccttccagttcatcctcacctacagcttgacttcccaagggcccatgccagctccctgtctacctgccagtgagttgatgagtctcggtgttagtagtaaaggcaggcgggaagcaagcagaagtgctactgggccttgagggtaagccaggcctcagccttctgaccccatcactaatgggttaataggaaaagcagtatccatctagtacagcctgccttttcaggaatagtgagtaaaagcaaagatgactaaaatacattaaagttttctgtaattgtctctaaggtctcccaacaaacatataccccatctgtttcaagctctgcataacctttcccagaagtcaagttcaggccctggcctcatggtgcctggcccaggttaagagtgctacctgatgatggagttaatacacgttgctttgacctctgactttaagatgtcctcccacttttccaccccgcaatctctagccctctctgggcacagcagcaattgggaactagttcctgtactgcctttatctcattttacaaaacaaacttctacaaagaagctggaaaggaaggaggagaaaggattatcatgcaggcacagggagggggcctagagaagagctctggcagattatgtccctcttaaaaaatgcaaccagaatcatcaacaaagtatcacctcaaaaatatgcaggagaaaaagaaaggaatcaatgttggggtggttggagcagaaggagccaaactgcccagaaggtgtcctctgaaggctgcgaggagcagactaggtcagccccagaggcagatggcacacacagaatggcaggattaccggtagctgcagatttattgacagaagccctagagactgggcctgctgcccagggaatgtgggggctcacttatcaaagactactggaaaatggctgagccggcaaccccttccactacagtgatgcctggttttcttgcacagcctgtaactctgcctgtaacaaggagaaaattaaagcaacgaatctggccaaatagaaaattaaggaaaactagaaacagctcctatggagagcagggattggggaggtgtagaggggctgatcctgaatgtctggaggatcaggaaaatgtaagcaattgtttaagggactggtaggaatcaagatctggagagagatcctcccgactctggtggctggggaatatgaactgtggagacatggtttcaaggactcaaaatgatatgacagcttaacatttacagttcagtgcagaggctctcaaagcatgatcccctgataggagtgtgagggggcaacaccatcacctaggaatttgttagatatgcaaattcccagacccactgaatcccagacaggtggggccagcaacctgtgaatctacaacaggctcaagtttgagaacaaatgacttagtgtaaggggctgctaattgatataaaatgttacctgtggtctattattttgtctgtagtgaatattgggcttgttaaggataaataaggtttgtgggctggtaagtggttcttctctacaaggttgcaacagctggcttcagttaacttcaaaaaggccctttaagcaaagacagtagtccccccactcatccatggttttgatttcagttacttatggtcaaccatggtccaaaaatattaaaaggaaaattccggaaaccagtcactcgttttaaattgtacaccattctaagtattgtttgacttgttctattttattattagttattgttgttaatctcttactgtgcccaatttataaattaaattttatcataggtatgtatgtatagggaaaaacataatatatatagggtttggtactatccgagggttcaggcatctacttgtggtcttggaacatatgccccgcagataaggggggactgctgtacaatgcaaaggacaaagattaaattatattagcaatctaggagcagaagggcaagactgcttttttaaaaaacagctaaaggtttaggaggttttattaatatttaaattgtattgaaaccacagctgcagcctttgactccagcatagagatatgcaaatatggctttcaaaagaaaggcaatttcagacagccctcaaagtaacaggaacaaataaaacaaatgattttgtaatttatctttattgactgatgttgcacaaggcacaggccataccctgtgagagtcagcaacagccgagctctctgaggagagaagagaaagccaggctggagggagaggcaggccgacccatagacaggtgacaggaaagacacagagcaggcagatgggagaagaagacaactaaattaaaagggaaggaaaataaaaacccagccctgggtcctgtagaccatctgatcttgctggctctcagcagcaacaacaataatcattaatgactatcatttgccacactactactaagtgccatgcactattcctcacatacaaatgaggaaaatgaagctttgagaggtcaagcaacttacccaaggtcacacaacaaaaggaaggggcagagcccagattcaaagatttgtgtgaggctgaagccctgtgctctttccagtgcattatgctgggaaccagtcctgggaggcagtgaataacaataaggttaatgggccgggcgcagtggctcatgcctgtaatcccagcactttgggaggcggaggcgggcaaatcacgaggtcaggagatcgagaccatcctggctaacatggtgaaaccctgtctctactaaaaatacaaaaaattagccgggcgtggtgtcgggcacctgtagtcccagctactcaggaggctgaggcaggagaatggcatgaacctgggaggcagagcttgcggtgagccacgatcgcgccactgcactccaacctgggtgacacagtgagactccgtctcaaaagaaaaaacaaaacaaaacaataaggttaatgattgaggggacactttgtgcccagttctgtggtattctgtatgggcatgcgtgtgtctgtgtgtgtgtatatgtatgtaactgtggaaaagagggtgaaaacctccatttctgaccttcaaattggttactatccaatgagtaaggcaagaaaagaaagccaaagaaaacttgcagaattctggtgtaaaagttcttttggggccgtgtggtggggccagctctgcctgttgtggaagacttctggtggaggcatctcagctggccttggccttgagtaaaatttagccagatgaaaaggaaagctggagattacacaggcccaggtgagagcctccagctgctagaattggaggaaggagcacctgattcagagagatgagaaaaggcaagagaatcctgaaaggatacatatctctgaccctttgtccccatccaatctccccagaccttccatcccaagcccaaacacaaccttacctgctgctccttttcaggcaccctggccaccaaatataggaacccataaattttgctcatactctatgttctactaggcaagtcctgatctgtcatctctacaggccccaatccttcccgctcacccctacagagccttctccaggttttctaggccagaatctctccccacttagaatactccagaagttttgctttatttgtgagactttattcaattgaagttacttgtgtgcatatgttatcctctctatttgactagaaggtccttataatcccttatgaccataattattttatctttgatataacccagctctgtaactagcagatactttgttaggcatccagtgggtttttcctaaatgaatgaagtaaaggatgaatgaatggactcagtgcattgaagggcttatccaactattggttccactctcaagacctttggaaaactagccatgttctggaatgctaattcccttcaatgcctttcgcccatttttctatgaccctgatttactccaaaaacaatataagggatctaagtgtccaagaatgactccttctaaacccacacctaaggattttctctctttttgtgtgtgtgtgtgtgagacagagtttcactcttattgcccaggctggagtgcaatggtgcgatctcagctcactgcaacctccgcctccagggttcaagtgattctccctgtctcaccctcccgagtagctgagattacaggcgcctgccatcacacccagctaatttttgtatttttagtagagacagggttcaccacgttggccaggctggtctcgaactcctgtcctcaggtgatccacccaccttggcctcccaaagtgctgggattacaagcatgagccaccacacccggcctctttgattctcttttgcctatcatgaagtctacccctttgtaattaattagaccaatgtccacccagacagaataacattttcccctatccatcagcgaggtcttctccgtgatggacattcaaggcagacagagagactgctgctgcaataactggggaaataattatggtgttcatgatgatttctttgcaggttcaaagcactagcccagccattatctctcccacttcactaggataaaattgctaaccccactttataggtgctaaaacaggtccagggccttgtcaaaggtcactcagtgagctggtggcagacctggaaataactagcctaggagtctcgatattcattaggccacagatggaaatgccctcattatgctgtctgggctatgtctgagagagagtcaactaactggactccagttaaatggagatatgcactggaagataagtttgtgactacagagtgtttttctctgcaatgctgcagcagttggcactggttaattccagagggtgtgtgtgtgtgtttgtgtgtgtgtgtgtgtgtgtgtgtgtgtgtttaaagcattatcacgcgtcctagatgagggaagagagggtgaatccaaggtaacacagacacacaggtaagcagatgtttgccatcttctcttgaaagtcatataaaaccaaatgacagtgtatattagcaggagaaactcaggaggctcttcccagctgttaggctatacgactctggaataagctagtacaaattaggtagaaagtctaggattgttcctagagcctggtggcgggaggtctttcctggaggcaaaggactgtggggctgtctcagggccttctgcagctgctaaagtgagaagcctgccgacgggatcatccccaagcccacagaagctctgaaagctatggaaaccaagatctgtacaggagccacttctggtttctaatgcctgagagattaaaatggaaaaaaaaattcccatggaaattcaagaatgcaagaatgttctggggccaggcacggtggctcatgcctgtaatcccagcacttttggaaggccgatcacctaagatcagaagttcaagaccagcctggtcaacatggtgaaaccccgtctctattaaaaatagaaaattagctgggcgtggtggtgtgcacctgtaatcccagctactcaggaggctgaggccagagaatcacttgaaccctggagccagaggttgcagcgagctgagatcatgccattgcactccagcccaggcaacaagagcaaaactccatctcaaaaaaaaaaaaaaaagttctacaacgtggccacaggtccgttctggctaaggcagtgatgtccccctcccaccaaagcccaaaccttctaacatcatcctaaagtgtgggaatcacctcttcacctcaggccagctctgggcttttctcagcctattcatcagcctccattagtcctcagctctgctgaggcctcagcagcttcccagtcccactgaaggctgtggggcatagaatgggcagagggcaggccgggcgtggtggctcaagcctgtaatcccagcactttgggaggccgaggcgggcagatcacgaggtcaggagatcgagaccatcctggctaacacggtgaaccccatctctactaaaaatacaaaaaattagccaggcgtggtggcaggtgcctgtagtcccagctacttggtaggctgaggcaggagaatggcatgaacccgggaggcggagcttgcagtgagccaagatcctgccactgcactccagcctgggcgacagagcaaaactccgtctcaaaaaaaaaaaaaaaaagaaagaatgggcagagggcataaaacctgagtccagaggtggtggttgcacaacattgtgaatgcactaaatgcccctgaattgtacattttaaaatggctaattgtatgttatgtgaatttcaatcgatttttaaaaaaaataaaactgagccacctttggggtggggagaggagctgggccaggctctgaggatttgagggttgaaactccttgcagggagtgaaatgaacgacaatggggaggccagtctggccctcccaactcctcctccaggaccagatgggaactggggctagggagaaaggcccaactggggcggcgccgggctctgggcagaagagaagcactcagtgaatgtgaggaggctgcagccgtcggctcatttgcatcataagtgattggttttccctgctcgtccctcatcaggacacaatggacagttgtttgctggcgcagcagatccatttaccaagggagagaggagacagagcacaagtgaccgatgggtaatagtgttgaagggtgggcagccgcctcccctcccctgtgctcccaggccactgggactcttgttctcacacaatgagaagggaccttagagagcaaatcaccgcttcactttatagaagaagagactgaggtgctgagaggaggtgagccttgctgtggtcaaacagcaagaacatagcaaaactaagcattttaaactctaacctctggatcctttttctttgagacaaggtctcgctctgtcacccaggctggagtagtacagtggcacaatctcagctcactgcaacctctacctcccaggctcaagtgatccccccacctcaactttctgtgggccacgacacctggctaattttttgtagagacaaggtctcactgtgttgcccacatttttctcgaactcctgggctcaagtgatcctccagccttggcctcccaaagtgctaggattacaggtgtgagccacagcactgggcctgtttggtttttgttttgttttgtttttttgagatagggttccactctgtcatccaggctagagtgcagtggtatgatcactgctcactacagcctcacactcctggttcaagtgatcctcccacctcagcctcctgagtagctaggactataagtatgtgccaccacgcttagctaatttttatttcttttattttttctagaaacagtgtttccctatgttgcccaggctggtctcacctgggttcaagtgatcctcccacctcagcctcctgaatagctgggattacaagtgtgagccactgcaccaggcctggattctaaactgtcatttgagggttcacttcgtttccccataataccttctctgtgcccttattcccatctctgtggtctcctgttcccggggccttcgtttccatcattctgtctcctgggtctatttctttttctctgtttctgttcctctctgtatcttttcctcttggtttccaggcagctaggataattactagacttttaattaacccctgccctaacaaaggtgggtctggcatgaggcagcaatttagcaagtgtcttggtttgttttggggataggtggggagagaaaaatatgtgttggggcctattataaaccaggcactaagacagacacataatgcactttatctcatttcatcctttcaatcttgcaaggtaggtgttcttagagacaaaaagggtgaggctcagagaggttaagtaacttgtgcaagggcacccagctagcaaattgtagttacctgactccaaaacctctgttcttccatctcacaccctggcacagggtctaactcagggtaaggggctcattgatttcaggcgcaagggaggcacaaagtcactgaaggagaccactgttttgttgctgtcctctaggcagcgactgcgtccccccagagccccctccttctctgagccccttctgcagcgtggcgaaatctcacaaatgcaagcttttgcccccagggaggtggggaggcagtgatcaagaaagaaacctgacaaacccagaccaaccatgggggtctccctcctgttaacacccctccctaacagccctcctggtggttccctgtctgcccctcccctttatgggtcaagcctgctggcgtctgtttcattgtgctgtgggggaaggggagagtcaggggtgagtgggtgtctgtgtgcatgaacataaggcctccgggttcattctgacactgaatgaaaaactcaccaattattcgtccagtctcattaatatgcagacagacatctgttatttaggagtcaacagcagaggcattttcttgtcgggaggggcactagtgtacagggctctcttgtctctccgctgctagcgggtagctactcaggaatcatcccacacctcccgacctggagcctcccctctctctgaccctcactcacagctttgaggacagcaagtaagggattgaccagacagagatggagggagatctgggaacctggctggaaggaaggaagcaggagaggagctgccttgtgtagaacaaactgagaacaaaacgctaaaccctttcctggggaagagaatgtggagttgggggagagagctgtgccaagagtgcctgccccactgggaatctcagggacatgacccctccccccacaccttcctcagcctgcaggacaagtctgagtgcatctgaagcagggagagggtcactatggcaacatgaagtcctcacccagagactgcagaaaacgtaataagaggagttcagaaaaatggagaccaagggacctcaatctttttttttttttttcaatttatttatttttttttattgatcattcttgggtgtttctcgcagagggggatttggcagggagggacctcaatctttggaggagtcactaaagctctcttcaggccccaagataggggtgggaacaagacataaccacctcctgctttctgtcttctgtcttcctccagcctttaagtcccagcacaaaatacccctccaagaagcctttcccaactccctcaggcccagcttagaagcacttaagccttggtgtcttggttgtaaagaataaaagttgacatcagctgagcaacacaatgaggtagatgtggtgatcagcccccttttctaggtgcaaaaactgaggttcagagaggtgctgggcctcaccaaagattccccagggaagaagcagcagagctcaacccaggccctgggacttctgcctctgaacctgaagctcttcccacgactaccccctgggagggccagagtcacaaggggaggacccttgtcagctgaagtgtttcaggagtttgattgagtcctctcttcccatccaccctgtccttccccctcctccctcctaggcaggcggattgcctaggttaagaaacactagtctgggcgaggtggctcacgcctgtaatcccagcactttgggaggccaaggcaggtggatcactaggtcaggaaattgagaccatcctggctaacacggtgaaacctcgtctctactaaaaaatacaaaaaattaggccgggcgcggtggcttatgcctgtaatcccagcactttgggaggccaagacaggcggatcacgaggtcaggagatcaagaccatcctgactaacacggtgaaaccccgtctctactaaaaatacaaaaaatgagccggacgtggtggcaggtgcctgtagtcccagctactcgggaggctgaggcaggagaatggcgtgaacccaggaggcagagcttgcagtgagccgagattgcgccactgcactccagcctgggcgacagagcgagactccgtctcaaaaaaaaaaaaaaaaaaattagccaggcgtggtggcgggtgcctgtggtcccagttactggggcggctgaggcaggagaatggcgtgaacctgtgaggtggagcttgcagtgagccaagattgggccactgcactccagcctgggcaagaaagcgagactctcaaaaaaaaaaccactagtctgggcgcggtggctcctgcctgtaatcccaacactttgggaggccaaggtgggtggatcacctgaggtcaggagttcgagaccagcctggccaacatggtgaaaccccatctctactaaaaatacaaaaattaggtcaggcatggtggctcacgcctgtaatcccagcactttgggagactgaagcaggcggatcatgaggtcaagagatggagaccatcctggccaacatggtgaaaccctgtctctactaaaaatacaaaaatggctgggcacggtggctcatgcctgtaatcccagcactttgggaggccgaggtgggtggatcacgttaggtcgggagttcaagactagcctgaccaacatggagaaaccccatctctactaaaaatacaaaattagctgggcatggtggcacatggctgtaatcccagctacttcaggaagctgaggcaggagaatcacttgaacccaggaggtgaggttgccgtgagccgagatcgcgccagtgcactccagcctgggcaataagagtgaaactccgtctcaaaaaaaaaaaaaacactaaaattagctgtgcatggtggcgcgcgcctgtagtcccagctactcaggagactgaggcaggagaatcgcttgaacccaggaggcagaggttgcagtgagccgagatggtgccactgcactccagcctgaatgacagagtgaaactccgtctcaaaaaaaaaaaaaaaaaaaaaaaaaaaacacagaaactctggcagccatcacagtgtgattatttgtttatttcattaaatgtttaacgaggctacattgtttcccaaaccaatgtctaatttgtgaaggaaacagcgcagagaaggaagctgggtgactcctgcatctggggtggggaagggagtaaggtcccctccctccatcctacagaggcctttgaggatcagcaacagtcccattccctcctcccacccactgagctcctcagcccagagccctcctccccagaaataaaacgtctggcaacccagacctgcagaaagggaccaaaaatccattcctggtggtattgaaaatgtattaaactttggggggtcctccagctgattgatttttctaattatgtttgctttagatggatatttaaatgcatttgcattccctgagctcacatggcaggatatggaggttggaggaaagagggggcacaaacactccacactctgcactttggtggttgcaggcttgaacctgctatacactgagaagtccaaagtggaaaagagaagccactcagctaaaaatcgcaagtcgatttttatggcaggtccttgtggggaaagggtcagtcctcagagacagatggagatccacctagctgggcctggagcccctgccctctcctgtacccttagccgaggactcagggtctttgagtcagtccctaaccaggtctcagtttgagggggtggttatccaagcacacttagataatttcaaatgccattgaagttatcctagaatctttgagactggctgagatgaactagtcccataggagaggttgggatagggatatctgatgatccagggagtggtgggtagggattcctttcctctcaagactggaacctggcataagggaaaggagaagctattttttattttttattttttaattttttttagagacagggtctcactctgacactcaggctggaagacaatggcatgatcatagctcactgcagtctctaactcctggcctcaagcgatcctcccagcttggcctcccaaaggaggaatcttggctgggattataggtgtgagccactgccgagaagctattattttaaatgacacacctcagagccaaatctcccagctccaacaccacatccagataaccatctatccaaaaaacaactctgatcacttcactctctgcctgaaattcctggtggcttcatcctctgaggatgatttcattcatcctcagaatgaaattctgattcctctgtggaccctgcagtagcctcagtgtaccttcctagccttgtcttctattctccctgccatgggagccccaacagtgctatgctcattctcatctccacgtgtttacacatgctgtaccctctgcccagagtgcctttcctaccccttccctgcccggaaaactcctcttcaaccctcaggacctggctcacaggactggcttctcaggctgcaaggagctcccatagcatcccatacatgtacaaatatccctcagccatggcaccctcacacctgaggtacctctcttccacactgggctggcctccaaaagcttagagactggtccttgcaatctcccaagccagtgtatgccacacagttggtgttcagtacatacttgctgaatgaatgaatgagggaggaatgggctataaatttgggtgggatcccagcagatagttgggtaaggtcagtgttctcttccagtgtgtctgggagaactggctagggctgggggagggaagggccagggatggttcctgggggagaatgtcaccgaaaagaggccagtgggaccagagccaggaagggaatacaggacaatctgaaaccagactcccgagaaaacagaccagtactgtctttcctgacaacaggcgctcagccgtcctctccaccgtctttcctttaagggacagggtaggggtgactctaacagctgatgctcccctgaaagccatcatgaaactcagcatgggaggagagaaaggtccctggcctgagcctctaaggagaccccagaatcaaactactgacctcttaggaaacttcacgctgtacaggggtagcttctgtgatgtggaggcttttgatgccttcttttttttttttttttttttttttggagacagagtcttgctctgtcatccaggctagaatgcagtggcgcaatctcggctcactgcaagctccgcctcccaggttcacccgggttcacaccattctcctgcctcagcctcccgagcagctgggactacaggcactgccaccgcgcctggctaattttttgtatttttagtagagatggggtttcaccgtgttagccaggatggtctcgatctcctgaacttgtgatccgcccacctcggcctcccaaagtgctgggattacaggcgtgagccagcgcgcccggcctgatgcctccttatccccacaacccccagggtaccagcaggctccaagccaggggtacagatggtgagcaggacccctcccacactagcagcaggcctggctgggctgagaaatgctgactaattatatgtcggtctgctctaaaaccccctaatggcctgagaattgcccacttcattaactaggatgaacagtcccaggatgtctccttctcccaactctgactcctaaaaggacacttctgatccaacctatggctttgtcctctgctctatctgtggacatggacaggaaagttccccagctgaggtctaactttccctcttactgctaaagattggtagattgatttttttaaaaagcaacaaaactaaagccacagccgttttccatggaggtgggctttattaggtgactgttgaggcaagggaggttctagggctggtggactgatggggggcaagggcttctccttgcttttgaatttagtgcatgttgcctagaggttagatgtgtgagaatagctgcagaagtgagaggagaggaaaagagaaggtcatagaatggacattttccttgggccagaaccaggatatggggactgggggtggagagggagggtactcttcacataggagactccaccaaggtcaccatttgataccagcttccctaacgcccacctgccccatcccagttcatgccccagatgccagccctgttagctccctcaacatccactggagaagaggagggaggaggagatgagaaacgatgatacccttctgccctccagctcccctgcccagaattgctcgctctgcctgccctaacttcccagccaggatcaggaggtcaggaagcctgggcgcaggcaggggaacaattgtgtccctcaccacccctcttcacactctgccctctcctagcccctcacatgaggttgcagcttttggctcgatccttgtgtatctcgccctcattcccccggtctggccgtcctgacagctgagcggatcgctgcattccccggcgtgagtcagttcggcgcagctgcctatggccacggccaagggaggccactgtagccacatggaagacatccctgacgctgcgctcagaggaccgggaggagcactcaacataggacacagcccccacctgcttggccagcacagtgccctggaaaaggaaggagtgcatgggagaaagttataggtcttagcctgcagcttagaaaggggcatggaggctgtgggctggggttgaatgtcaaggctgggggcagtcaagggatcaggtcagaggtcacagaccagaacaaaggatgagccaggtaaggtgttttcaaatcagagactgaaggggcagaggtgacaggtctaggctgggatgaggtcagacgtcaagggtcccacctgctcatgtgtaacagggataagcctctgcttggacagctccctcagtgtggccaggtcagtccgcatgtccagtttacagccaaccagcacaaccttggcattggggcagaactcttgagtctctccttgccactatgtaggagaagaaaagagaattgtggaggggtggagggaggcacagcgtgagcctttgtgccatgccagcagtgtgccctctcttcctcaagcacgcagaaaccatgtatccctcaggactctcccacatgaggaaggaggtgagaatgctaggcttggtttgagagcaggatggggtgagagagaagcagcggggaggagagggctggcgtgggcctgtggactcttctccctcagtggctatgaagggtctgccagcctggaaacttcccattcccactcgcctttcccatactctctcctgggaacaaatttatcccaccctcccctcccccacctgggaactggggctgccctagctgacttctgtccattcacccctggcttttgtcatggaaactggggcctggagaggaggccacagggctttcctgagctccaggatcaggcttcccatcccatcccctccatctgggactcccactgccaaggagttggaagagcagagaaggaatcctgtgagtgcttctcccttcactcccccacctcccctagctgctgtatccagctctgaggactttcaggaaaggggtggctgggagggatgggggctggaggcaggggtggcagggatgagagcttcactcgctaaccagctgatggccataccccagccatcactccctcctcctggcccatgttatgtcaggaccatggtggtctggtccccctcagtctagctgccctatttccccaggctcccaccttcttgagaacactgtccagtgtttctggtcggctaatgtcgaagcagatgagcacagcatcagaatcaggataggccagaggccggacattatcatagtaagaggaacctgaaggaagacagggcatgaggggcctgagtggatgggaggcctggaagggaatgggagaccttacagatcctggtcaagggatgctgggagcagagaatggaaaagagcaaccattaatacccatcattaatacccatcatggagtctgggtttcccaagtgggggagcgggcagaagggaggtcacatggtgggaaacggctctctgcttccctagtggtttaaaaagtaataatttttattcttgactgaggtttggacccctttgagaagctcataaaatctatagacattctcataaaatgttgcatgtgctttctgggctttggcagtctaaagccctgaagggaactcttgtaaaaataaaaaccctccttatgttgggaggaggaaaagagcagtggcagggagagcactctcccattctcctgggacttgacctcagatttatctagttcaatggagagaaacagcctcctgaccacccctcactcaagacattctaaatgtcttttctgcttttttattttttgagatggagtctcgctctatcgctcaggctggagtgcagcggcatgatctcggctcactgcagcctccgcctcccaggttccagcgattctcttgcctcagcctcctggtagctgagattacaggcacgtgccacacgcccgattaatttttgtatttttagtagagacggagtttcaaccatgttggccaggctggtctcaaactcctgacctcaggtgatccccccaccccagcctcccaaagtgctaggattacaggcgtgagccaccgtgcctggcctctaaatgtcttttctaaacccagtcttatctctgaaggaacatgttccaaattaaaagccattccttcccaactttcccaggtaggcaggacccagctgaggggctcagatcctaggctttctccttcaggacatggtttctgtcaggctctgcaagttctacctcagtttccctggattttagcagaatattgatttttcccttcctgttggaattggatggatgggctgggcttacctggaacctaagggtctaaccaagggaggggacagagtgggccgccttggaagtcagggtgacccccagggacttggctacctgaagtgtcccacatgttgagctcaatgcggcgcttgtcgatctcaaagctcgcagtgtagttctcaaacacggtggggacataactctgcagacacggatggatccagatgagatcatctctccaagtcctcaccccagataaatgccctcctcactcccacaccccgtccagcccagcccctggacacaagttggtctgggattgggcaaaggggagatcccgggagctcctgcccccccccccccccccccggagaatctctgctaagctccaagacctcctcctgttcccttctggtcccttcagccccctagttcttcccctctccattgcccaggacctccgacattcccctcgccctccctcccggatgctctggttctcccaaaccccatctatctcaccctgctccctaattcctcccaccccaatcctctgcagtccttcctgagccctcaaggcctccccgcgtcctccgaccccctgtctcttgtccctggttacccgagtacccattcccatccgtcgccagggcccctgtcacccaccccccagcagccttagcgtccccctcccaagacgcaggtccctcaccccgggataggcgtccttggcgaacacctgcagcagcgccgtcttgccgcactctgcgtctcccaccaccacgatcttgcagcggccgctctgcccctccatggtcccggctacgagccggtcccccacgccacccctctcccggtcccctccttcgcctctcccgcccctgcaactgcagccgctcgggccgccaacaccggcatctcccgggcccgcgccgagcccccgccccgggtccgcggccccccctccgccccgcccccagccagcgccgcgccccccggccctcctcccacccccgcagccggggtcggggccagaagatctggcggagccctgggaacagaggcctcagagccggggtccagcccgccggtgtggtctgaggggcccctgccggtttgggacaggccgaactgggcttatttgactttctcggatataagggcaggtcagagttcaagcgaagttcttaggggtagaatatgagcggcacaagcgcgaagctcgggcctgctgtgtacccacgcgtgcacgcaggtgtaccagtgcggacaagagctggggcagccatccacttcctgaacacggcgggagagagatgctaaggggaaggagggagcctctttggttttctctcccgcgtccgcctatgtcctggcagggggtcttggggaaatgggagggtgaaccccagcacacccacccggacggtggtgacatcatagctttccgtccccatggcaacgggcagccgggtctccggttacattgacttaaccgccggcctagactagcagagaagcgtggactgagttcctccagccaagcactgggtggaaagttttgggggagctgcgtcctctggtggatgcttggggacaaggagataaggaagagaaagaaccaaccgccagagttgctctgctggagccagagctaaacccaaaagtcaggcttgattaagagtctgacaataggccgggcgcggtggctcacgcctgtaataccagcactttgggaggccgaggcgggcggatcaagaggtcaggagatcgagactatcatcctggctaacacggtgaaacccgcacggtgggcgcctgtaatcccagctactcaggaggctgaggcaggagaatggcgtgaacccgggaggcggagcttgcagtgagccgagattgcctcactgcactccagcttgtgcaatagagtttcgaaaaaaaaaaagtgcttttttatatcgaggcaattcgagtcaataatatatgctgcaaataattctgtaaagataactagaagctgggcgcggtggctcacgcctgtaaactcagcactttgggaggccaaggcttgcttgcgtgcaggagtttgaggccatcctgggcaacattagcgagaccctctctctagaaaaaaaaatcaaaacttagctaggtttggccactctaggcacgctgcctatggggtagccctgctctgcaaagagcagtaaaacataaagttagccgggcgtggtgacacatgcctgtggtcccagctattcaggaggctgaggtgggaggattgcttgaagccgggagtttgaggctgtagggagctgtgatcgccccacctcgctcagcctgggtgacagagtgagaccctgtatcaaaaaaataaaaatataaatataactagagcacgcagcatcatcactatgttacagaagggaaaatgaggaacagaacgttaacaccaaagtcagaaagttttaaaggcttggtctccatgcttctactttgccactgcaagaccacagtgaattaagtctcatccctgcctgggttagatgtcagagcctgagacacaatgtagttggactccagtccacaggtggctgactccaaatctgatatgagttaactccaaatctgatgtaagttcaagttttgggactgttccttaactttttttttttttttttgagacggagtctcgctctgtagcccaggctggagtgcagtggcgcgatctcgactcactgcaagctctgcctcctgggttcatgccattctcctgcctcagcctcccaggtagctgggactacaggcgcctgccaccacgcctggctaattttttgtattttttagtagagacggggtttcaccgtgttagccaggtgaatctcctgacctcgtaatctgcccgcctcggcctcccaaagtgctgggattacaggcgtgagacaccgcgcccggcctttttttttttcgagatggagtctccctctgtagcccaggctggagtgcagcggcatgatcttggctaactgcaacctccgcctcctgggttcaagcaattctccagcctccgcctccctagtagctgggactataggcacctgccaccatgcctggctaatttttgtagttttagtagagctggggtttcaccatactggtcaggctggtctcgaattcctgacctcaggtgatccacccacccgcctcggcctcccaaagtgttgggattacaggcgtgagcacggcgcccggccctttttttttttttttttaacagggtctcactctgttgcccagactggagtgtagtggcgcgatctcggctcacctccgcctcccaggctcaagcgattcctctgcctcagcctcccaagtagctgagattacaggcgcgcgccactaccgcccggctaacttttttatttttagtaaagacggggtttcaccatgttggccaggctggtcttgaactcctgacctcaaatgatccaccagcctcggcctcccaaagtgctgggattacaggcgtgagccaccgcgccaggcctatcccttaaaatagtttttaatttgaataaggtttactatgaataaataaatcacagtcggcttgatcccaagagcacagacgttcctggtgccccttttcgtgctctcccagcttgcgccactatggccctggccctttaaggctgagcgcgaggccccgcctcgcccggcgccccgcccctcccgctggatcccgcagccgcggctcttcccgacgcgttccgacttccccagctgtgcactctccatccagctgtgcgctctcgtcgggagtcccagccatgtccgacgagagagaggtagccgaggcagcgaccggggaagacgcctcttcgccgcctccgaaaaccgaggcagcgagcgacccccagcatcccgcggcctccgaaggggccgccgccgccgccgcctcgccgccactgctgcgctgcctagtgctcaccggctttggaggctacgacaaggtgaagctgcagagccggccggcagcggccccggcccctgggcccggccagctgacgctgcgtctgcgggcctgcgggctcaacttcgcagacctcatggctaggcaggggctgtacgaccgtctcccgcctctgcctgtcactccgggcatggagggcgcgggtgttgtgatcgcagtgggcgagggagtcagcgaccgcaaggtgagcgggttgcgtagggcagggcagggctgcgcaggccactgggcagtggggcacgagtgggcgagcgccgggggtgtggcagggcgggagaaactggcgcggacctgggtgcacgagcgtggaaagcgtagccaaggaacttgtgtttgggggctcctggagagcggcatttatgtggggaggggagacgaaattatcgccccttccccaaccattttaagttgtggccgccgcccagaagctgtgctggtgggggggaaaacaataaggtgcccatgcgcatgcgcacaaccacactaccgtccccacccaccccccccccccccattaaaaccacacctgtacccctacccaccaaacactctctgggtaattgtggtctgtgactatgagtgacggttagtgccccctttccccgagggagcttgaggggctatgtcgtcggggttgggcgggggcacagcggccgtgccagagtcctggtcacatgcagccccgtggtctgtgggggtgtgaggcggcccctcccaaagcaaggccaaagagacgagacacgcccatcacggaggagagagagcctttgctaccccaccgccaccagccttacaccgccgatctgattttggggtgggggaggcgggattgggtcatccgatctttgtcttgggctctgtgtctcccgtgactgcagtatctcctcctcctgtgactcagccctcagccttcgggccacgacccggggctgcccttgggaatgcctggggcggggagtggaaggggggacccacctctgccttcctcctgcagaggacccccacttcagaaaccccagtgccaggggtttggactggaacggagaggtgcggcgccttgaactggttggccaagtctgcaggcctgtttctccttctcatttatcattaatcttggccacaaccctggacaccagagagctcaaaatgatcagctttttgagagacctgggatgaggcctcagcacgccatttgtttagaggtttcttttttttttctttttctttttttttttttttttttttagacggagtttcgctcttgttgcccaggctggagtgcaatggcgcgatctcggctcaccgcaacctctgcctcccgggttcaagcgattctcctgcctcagcctctcgagtagctgggattacaggcatgcgccaccatgcctggctaattttcgtattttttagtagagacaaggtttcaccattttgggcaggctggtctcgaactcccgacctcaggtgatctgcccacctcggcctccctaagtgctggggttacagacataagccactgcgcttggccaggagtttcctttttaaatcagacccctcaatgagaggccccacagatgcagcctcttgcagacctgccagcccaattctggagccaggtttgttggattcatcctgtatgcaaacagcttctccttaaggctttcctctgaattcagctctggccccaccctcaaactgacttctaaatgatcccactcttgagcaggcgtctaagaggaatattttcgggaggtagttgtagttcatgttactgctgaaggccacccacctcacctcccctccatacactttccgcctggtaaatacaggatatcctgtccagggcaagaatctgatgtaagagcctggattctgcggggagggcccttccctctctctccctcctcctccctcctggtgtctgggttggggaggggtcatggccctgatttggatggcctgagggttagcatgagccagggtaagtgagacttgttctgggtcaaatctgggactggccatgaccctaaatgaccaatgcactcctcgcagctctcctgggttgttctgtatctgctagtcctgagtccctgggtggagggcttccgttcttgttctccagacctcatctcaggccagaacttggaaggaaagaccccagcatgccctcagttctcgtattcagtggagtgtgggggcttgaggacatgaaaaagggcgtaagtggcagtcccatcccccttccccatggaccctaactcttgttaatatacagaattcccatcattcctggcagggatcaagacagacccagattgtcccagaacagcacccacacctccctcttcatgctcttcagagagcgcagagaagtcttttctcctgacgctccctccttttccctgcccttcccttgaccccacttgctaagctggagagaaaggttctgttatctttgtcccctttccctcctgcacagaggctctcgtgggggtgggggggaagccttttactgctgcgtaggcctctgtagcccttcttgtctgttgcccctcctgcaccatctctgagtgaagatgttttctgggctcccagtgctggctcaaacacacttctcccgaggtgaccacaccctgctgtaagcgctcagagaactttgtctgcactttggtatggccctgaccacacagtgccgtcttcttttggttgtgtgacttccttgctgctattatattattattattattattgttagctaacattattaagtgcttattatgtgccagacattgtgctaaaattttttttttccatttggaaaaactgccctaattgacagataagaaaactggagctggaaaagtggagctcaaaaagattaagtaatttttatgcatccgaagtcacacagtcagtaaacagttgagaccgcttttgtaaccacagcagtttgatcttttccacaatacttcatgctgccctaactgtaagcttcttgcattcagggatcttacacaatagtgacatgtaagatctgtaagaagatgataaggatagtatctgcctcaaagaactgatgtaagggttaattaagcattatatataaagcacttgaaatcgggctcggccctcagtcagcactcagtaaaggtgagttgttattgttgttgatgatgatgttattattattattaccatgactattgctgcttctcctgctacttcatttggaagaaggtggccttgtacctagggggaaatcagtaaatagcctgtgggtgaatgaatgagtgactgaatgatactcttctgttcttcaaggcaggagaccgggtgatggtgttgaaccggtcagggatgtggcaggaagaggtgactgtgccctcggtccagaccttcctgattcctgaggccatgacctttgaggaagctgctgccttgctcgtcaattacattacagcctacatggtcctctttgacttcggcaacctacagcctggccacagcgtcttggtacacatggctgcaggtgacaggtcccctcactttatcaccccttaccccacccagatttccttccaggccccttccctgcagcctgtctgggttgttgtcatggcaacaccaggctgccttggcctgtggctcccagaggcctctgctgtgtagttgccgtggtaacattcaggcaccaggtctagtctggtgtgctatccttagcaacgtgccctcaccccacacccccacctctctagctaccttccccaccacttctcagtcatggaaattagacacggccctaaaatgagcgtaggcaaaatgaaggtgacaggctgagtccctgggaggctagaatggagtggttggtggccagggcaactccatatcccctgcttatgggtgtcttgctgcagggggtgtgggtatggctgccgtgcagctgtgccgtacagtggagaatgtgacagtgttcggaacggcctcggccagcaagcacgaggcactgaaggagaatggggtcacacatcccatcgactatcacacgactgactacgtggatgagatcaagaagatttcccctaaaggtggggggcataatatgggagggggtagggaggcacaggacagggaggggagctccagatctgtggatcctaatgttgttcttgggttcccctactctatgacaggagtggacattgtcatggaccctctgggtgggtcagatactgccaagggctacaacctcctgaaacccatgggcaaagtcgtcacctatggtgagttagtgggccagggatggagagagcatgtgagggcaggagggagggtctaaggggtgggatatagaggccagggcttttgaatgaagaaggggtagggactcaggtgctctgtagacgatcagggttaggaatggtcctgtatgctgcattcagattgctgactcttgggtaccagctcttttcattctctgtcacaacttttcatatgagtgatagtaaattctacattcttttttttggttttttttttgagatggagtttcgctcttgtcgcccaggctggagtgcaatggcatgatctcggtcagtgcaacctctgcctcctgggttcaagcgattctcctgcctcaccctcccgagtagctggaattacaggtgtctgccaccacgcccaactaatttttgtatttttagtagaggcggtgtttcaccatgttggccaggctggtcttgaactcctgatctcaggtgatccagccgcctcagcctcccaaagtgctgggattatagccgtgagccaccacgcctggccaaattctacattcttgtttggggattattcttgaacaaccagcctgccttctttctgtcctacctccctgagcatcttaggcagggtgcattttcatttaaaaaagtatttcatacaacaaaataagccaggcatggtggctcatgcctgtaatcccagcactttggaaggccagggcaggcagatggcttgagcctagaaattcgagaccagcctggtcaacatggtgtaccttatctccacaaaaaatacaaaaattagccaggtgtggtggcactgtactccagcctgggcaacagagcgagaccctgactcaaacaaatgaacaaacaagcaaatacataaagattagagcaatttttttttttttgagacagaatatcactctgttacccatcctggagtgcagtggcgcaacctcggctctctgcagcatccacctccctggttcaagcagttctgcctcaggctcctgagtagctggaattacaggtgcccatctccacgcctggcttttttttttttctttttcttgagacagagtctggctgtcacccaggctggagtgcagtgacacgatctcggctcactgcaacctccacctcccggattcaagcaattcttctgcctcagcctcccaagtagctgggactgcgggcgcacgccaccatacctggctaatttttgtattgttgtgggtttttttgtttgttttgttttgttttttttttttgagacggagtttcgctctttttgcccaggctgaagtgcagtggcgcgatctcggctcactgcaacctccgcctcccgggttcaagcgattctcctgcctcagacttcctgagtagctgggattacaggcatgtaccaccatgcccagctaattttgtattttcagtagagacggggtttctccatgttggtcacgctggtctcgaactcctgacgtccagtgatccgcccacctcggcctcccaaagtgctgggattataggcgtgagccaccatacccggccaacacctggctaattttcgtattttttagtagagacaaggtttcaccattttgggcaggctggtctcgaactcctgacctcaagtgatccccccaccttggcttcccagagtgctgggattatggatgtgagccatagcacccagcccctagagcaatttaaagtcagccagggttggtttgggcctggtaaccagcagtttgagaattatccaatcactccctggcactggtgtggagaattgcaaggggatcacagggaacagagagcacagatgcagacacacagggatgtgcctgggggggttcacaggctatgtcaccttgtccttcaggaatggccaacctgctgacgggccccaaacggaacctgatggccctggcccggacatggtggaatcagttcagcgtgacagctctgcagctgctgcaggccaaccgggctgtgtgtggcttccacctgggctacctggatggtgaggtggagctggtcagtggtgtggtggcccgcctcctggctctgtacaaccagggccacatcaagccccacattgactcagtctggcccttcgagaaggtgaatgtgaggactttgcagggagggcttgggtaggactcatgaaggctggggtcccaaggggcagattcctggggaagaggagggctgcctgcatcacactggcccctgttggatgagggttggatagcactgggagccgcatctttccttcctccccaggtggctgatgccatgaaacagatgcaggagaagaagaatgtgggcaaggtcctcctggttccagggccagagaagcagaactagggcaagtggctgtgagaccctagagaccagcgaagggagaagttgggaagctacgttctgttggccaccagacttgcatttcagcctctgtcataatgctctgccctccctcccccgaagttctctgtggtgatgaccgctctcccctgcccctccccgcttcctgacctctgaagaggttgggaagtgaccatttggatgtctgggccctgccaaggcgacagggagggtcagagggaggccggctgcttcctgcccccaccctttccccgggcctgctgtgctgcttttgtgccaaggttagccagtcccccctgttgtgttccatgtgctttcacctctgcctcatctttcctcccgtccctgccccgccacctccccaaagaattgaaacgtcagctcaggatatggggccaatctctgtgagtccagcatgtacctgtctctccctagtgtcccttcagcctgggctgaccagtgcccgcctctgggcttgaccagttcccaatctcgtcctctgtccccaacttcttaagcacaattgggcttcttccatctccaggttttctgccattcttaaccaaggctgcctcttccaacagggcgggaatcagacctactcccctaggtcacaactctgggaaggatacagagcccccacccttcactgagttctctggatttgttctcagtgccttagcaacgaaaacctgtgcttgtgtgtgtgtggcggcggggagggaggatcctgtttcccacctccttctcctcccctgtactccccagtgccttccttgttctggtggagctggggtttctctcctccccagtcccacaacactgccaaaaatctgtgtatgtgccattgggtggggcagccccaagcctcctggggaggcagggcaaaaacaggtgccctcatcgtggtctgtgccatgtcccgtctctatggtggttgaggagaaaggcggggaagcttcctcagccttgcagatatgtgtggcatttactagccagagctctgaaaggcagtgctgtctgtttcttgtactgggaccaaagtaaaaatccaagcacattccccttgcagttaggggaggccctactgccttctcaaagcagagaggcagcttatcaaactcagcccaaaactctgtttacatgggtggggagatggagcagggaagtacagagtgggatggtcaggacctgggccattgcaaccaaaatggggacttcctgggtagggaggtcactccctctactcactgagctaggattagggagggttattgccccaaccattgcaatgggaggtggagggacaggctcagcctcctcattgtctaaatgaggcctaaatgtgtgaagtgcgatttctgcttttgtgtaccccaccaccccattaccacagctgcctttgtgtgtttgtgtcaataaaaagccaaaccctgggtcctgcttgttgcctctgagagtggagggaaggtgagctcctggaaggctagtgctgccagcagaagatctgggctgcttcctgccccctgcctctttccatgcccaaatcacgtttcctttcatgagtgaaatgaggaagaacatcatggcatgcaggctctttttactgttctgggcagtgttttgcaatgtgtgacccctccgcactgttggtgaacatacagacctcctatatgggcacccaagcccaggccagtgtgagaaccttggcgggggggtggggaggatgagaaggggaggcccctagcctgactcagaggtgaagactgctaggccctgctgtccttggggtacgactgtcagggcctctacctccccgcccccgcgggtgggcttctggaagtggatctccaggacgtcatgcagctccgggccatccaagatatcaggaatgttgagcaccagtaccgagcggggaactggctgcgacctgatctggaaaaggagccaggagatggcaaaggctcatgggagaagggctgggggctgggggtagggcaggcagtgccccatgggggtatgaggtcgggaggcctggagaggctgatggtgggtaggtaggtcagtggggtttggggcctctgatccttcattgggcaagttcctggcaggcagacaggttacccagcccagcccctgttcttcacccctcctgcttacctcagccttctggatctccccattcacatacggagagactctcagagggacttgctgcccacccagtggcactgtgaactggccgatttggcacagacgctgagccactgtggggagcaaggaggggtgggcctttagcatcaagccctctcctctccctgcaccataatcttgcacaccctataccctctcccctgcaggaggcctgcatagccctcacctccatccctagcaaaccccagcatgacactccctggcagtagctcccgaacgtccacatcgccacctccgttcctagtcttgccaaagaagatctctagcttgtccagcagctcctcctcactcagcctgaggctggcaggaaatccagtgaccaacaccctccggccactcaactggctggacacctagggggagacagcaaggggtggtcccagacacagctccacttccccatgcccaggtgcatggcatgctttgcatgccccaggattctgtcataccatcacctggatggtggtgaccatgggcagctccaagggctggacctgcacccgcagccggcactcctccatgttgatcgtgtgctccttttgttgcagcacctgctcagccactgggtggtgggaaacagggtcagtactgggaaccctccccacctccctcctgaggctccccatgagcttacctttggggtcatcaaaggtgatcagagcagagcccgcaagcagagggcagtggatccgcaaattggaaactaaagacttaggcacttccgggtcctgctgggtgtgtcctcggaataccagggggatcttgggcactgaaaatgggacctggaagtaggggaggggagtaggagtgttcctcactcccacctaggaggaaggcatctccgattccattccattgatgctgagtgccggagataaatacatttactgaaagaaaagctggataaatgagtggatgaatgcaggtctggtccatacagggcaatttacatacaacatcttaatttgtcctgggaggtgggtagctctactttacagatgttcaagtaacataatggaggtcacacagcaaggaagtagcagagccaggaataaaaccgagaactccccagctcttttttgtgtttctttgttttaaagggagtcttgctctgtcacccagctggagtgtagtggcatgatcttggctcactgcaacctccacttcccaggttcaagcaattcggactcagcctcccaagtagctgggattacaggcatgtgccaccaaacccagctaatttttgtatttttagtagagacggggtttcaccctgttggccaggctggtctcaaactgacctcaggtgatccaccagcctcggcctcccaaagtgctgggattacaggcgtgagccactgcgcccggcctaatttttgtatttttagtagagatgggatttcactgtgttggccaggctggtctcaaactcctgacctgaggtgatcaggcccccttggcctaccaaagtgctgggtttacaggtgtgagccaccgcacccggcctcgccagctcgtttttaccaaacaacaccagatcctttagggggggttattgcgggcggatttttgaaacggagggctggtgataggctttttataggtatgcatgtatatggttctagcgtagtggattttgggaattgcggt

SEQ ID NO: 2 - protein, 1863 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 3 - DNA, 10987 bp, Homo sapiens
  CDS (229)..(10485)
  ref <308>: DATABASE ACCESSION NUMBER: U43746.1
  ref <309>: DATABASE ENTRY DATE: 1996-09-03
  ref <313>: RELEVANT RESIDUES IN SEQ ID NO: (1)..(10987)
ggtggcgcgagcttctgaaactaggcggcagaggcggagccgctgtggcactgctgcgcctctgctgcgcctcgggtgtcttttgcggcggtgggtcgccgccgggagaagcgtgaggggacagatttgtgaccggcgcggtttttgtcagcttactccggccaaaaaagaactgcacctctggagcggacttatttaccaagcattggaggaatatcgtaggtaaaaatgcctattggatccaaagagaggccaacattttttgaaatttttaagacacgctgcaacaaagcagatttaggaccaataagtcttaattggtttgaagaactttcttcagaagctccaccctataattctgaacctgcagaagaatctgaacataaaaacaacaattacgaaccaaacctatttaaaactccacaaaggaaaccatcttataatcagctggcttcaactccaataatattcaaagagcaagggctgactctgccgctgtaccaatctcctgtaaaagaattagataaattcaaattagacttaggaaggaatgttcccaatagtagacataaaagtcttcgcacagtgaaaactaaaatggatcaagcagatgatgtttcctgtccacttctaaattcttgtcttagtgaaagtcctgttgttctacaatgtacacatgtaacaccacaaagagataagtcagtggtatgtgggagtttgtttcatacaccaaagtttgtgaagggtcgtcagacaccaaaacatatttctgaaagtctaggagctgaggtggatcctgatatgtcttggtcaagttctttagctacaccacccacccttagttctactgtgctcatagtcagaaatgaagaagcatctgaaactgtatttcctcatgatactactgctaatgtgaaaagctatttttccaatcatgatgaaagtctgaagaaaaatgatagatttatcgcttctgtgacagacagtgaaaacacaaatcaaagagaagctgcaagtcatggatttggaaaaacatcagggaattcatttaaagtaaatagctgcaaagaccacattggaaagtcaatgccaaatgtcctagaagatgaagtatatgaaacagttgtagatacctctgaagaagatagtttttcattatgtttttctaaatgtagaacaaaaaatctacaaaaagtaagaactagcaagactaggaaaaaaattttccatgaagcaaacgctgatgaatgtgaaaaatctaaaaaccaagtgaaagaaaaatactcatttgtatctgaagtggaaccaaatgatactgatccattagattcaaatgtagcacatcagaagccctttgagagtggaagtgacaaaatctccaaggaagttgtaccgtctttggcctgtgaatggtctcaactaaccctttcaggtctaaatggagcccagatggagaaaatacccctattgcatatttcttcatgtgaccaaaatatttcagaaaaagacctattagacacagagaacaaaagaaagaaagattttcttacttcagagaattctttgccacgtatttctagcctaccaaaatcagagaagccattaaatgaggaaacagtggtaaataagagagatgaagagcagcatcttgaatctcatacagactgcattcttgcagtaaagcaggcaatatctggaacttctccagtggcttcttcatttcagggtatcaaaaagtctatattcagaataagagaatcacctaaagagactttcaatgcaagtttttcaggtcatatgactgatccaaactttaaaaaagaaactgaagcctctgaaagtggactggaaatacatactgtttgctcacagaaggaggactccttatgtccaaatttaattgataatggaagctggccagccaccaccacacagaattctgtagctttgaagaatgcaggtttaatatccactttgaaaaagaaaacaaataagtttatttatgctatacatgatgaaacattttataaaggaaaaaaaataccgaaagaccaaaaatcagaactaattaactgttcagcccagtttgaagcaaatgcttttgaagcaccacttacatttgcaaatgctgattcaggtttattgcattcttctgtgaaaagaagctgttcacagaatgattctgaagaaccaactttgtccttaactagctcttttgggacaattctgaggaaatgttctagaaatgaaacatgttctaataatacagtaatctctcaggatcttgattataaagaagcaaaatgtaataaggaaaaactacagttatttattaccccagaagctgattctctgtcatgcctgcaggaaggacagtgtgaaaatgatccaaaaagcaaaaaagtttcagatataaaagaagaggtcttggctgcagcatgtcacccagtacaacattcaaaagtggaatacagtgatactgactttcaatcccagaaaagtcttttatatgatcatgaaaatgccagcactcttattttaactcctacttccaaggatgttctgtcaaacctagtcatgatttctagaggcaaagaatcatacaaaatgtcagacaagctcaaaggtaacaattatgaatctgatgttgaattaaccaaaaatattcccatggaaaagaatcaagatgtatgtgctttaaatgaaaattataaaaacgttgagctgttgccacctgaaaaatacatgagagtagcatcaccttcaagaaaggtacaattcaaccaaaacacaaatctaagagtaatccaaaaaaatcaagaagaaactacttcaatttcaaaaataactgtcaatccagactctgaagaacttttctcagacaatgagaataattttgtcttccaagtagctaatgaaaggaataatcttgctttaggaaatactaaggaacttcatgaaacagacttgacttgtgtaaacgaacccattttcaagaactctaccatggttttatatggagacacaggtgataaacaagcaacccaagtgtcaattaaaaaagatttggtttatgttcttgcagaggagaacaaaaatagtgtaaagcagcatataaaaatgactctaggtcaagatttaaaatcggacatctccttgaatatagataaaataccagaaaaaaataatgattacatgaacaaatgggcaggactcttaggtccaatttcaaatcacagttttggaggtagcttcagaacagcttcaaataaggaaatcaagctctctgaacataacattaagaagagcaaaatgttcttcaaagatattgaagaacaatatcctactagtttagcttgtgttgaaattgtaaataccttggcattagataatcaaaagaaactgagcaagcctcagtcaattaatactgtatctgcacatttacagagtagtgtagttgtttctgattgtaaaaatagtcatataacccctcagatgttattttccaagcaggattttaattcaaaccataatttaacacctagccaaaaggcagaaattacagaactttctactatattagaagaatcaggaagtcagtttgaatttactcagtttagaaaaccaagctacatattgcagaagagtacatttgaagtgcctgaaaaccagatgactatcttaaagaccacttctgaggaatgcagagatgctgatcttcatgtcataatgaatgccccatcgattggtcaggtagacagcagcaagcaatttgaaggtacagttgaaattaaacggaagtttgctggcctgttgaaaaatgactgtaacaaaagtgcttctggttatttaacagatgaaaatgaagtggggtttaggggcttttattctgctcatggcacaaaactgaatgtttctactgaagctctgcaaaaagctgtgaaactgtttagtgatattgagaatattagtgaggaaacttctgcagaggtacatccaataagtttatcttcaagtaaatgtcatgattctgttgtttcaatgtttaagatagaaaatcataatgataaaactgtaagtgaaaaaaataataaatgccaactgatattacaaaataatattgaaatgactactggcacttttgttgaagaaattactgaaaattacaagagaaatactgaaaatgaagataacaaatatactgctgccagtagaaattctcataacttagaatttgatggcagtgattcaagtaaaaatgatactgtttgtattcataaagatgaaacggacttgctatttactgatcagcacaacatatgtcttaaattatctggccagtttatgaaggagggaaacactcagattaaagaagatttgtcagatttaacttttttggaagttgcgaaagctcaagaagcatgtcatggtaatacttcaaataaagaacagttaactgctactaaaacggagcaaaatataaaagattttgagacttctgatacattttttcagactgcaagtgggaaaaatattagtgtcgccaaagagtcatttaataaaattgtaaatttctttgatcagaaaccagaagaattgcataacttttccttaaattctgaattacattctgacataagaaagaacaaaatggacattctaagttatgaggaaacagacatagttaaacacaaaatactgaaagaaagtgtcccagttggtactggaaatcaactagtgaccttccagggacaacccgaacgtgatgaaaagatcaaagaacctactctgttgggttttcatacagctagcgggaaaaaagttaaaattgcaaaggaatctttggacaaagtgaaaaacctttttgatgaaaaagagcaaggtactagtgaaatcaccagttttagccatcaatgggcaaagaccctaaagtacagagaggcctgtaaagaccttgaattagcatgtgagaccattgagatcacagctgccccaaagtgtaaagaaatgcagaattctctcaataatgataaaaaccttgtttctattgagactgtggtgccacctaagctcttaagtgataatttatgtagacaaactgaaaatctcaaaacatcaaaaagtatctttttgaaagttaaagtacatgaaaatgtagaaaaagaaacagcaaaaagtcctgcaacttgttacacaaatcagtccccttattcagtcattgaaaattcagccttagctttttacacaagttgtagtagaaaaacttctgtgagtcagacttcattacttgaagcaaaaaaatggcttagagaaggaatatttgatggtcaaccagaaagaataaatactgcagattatgtaggaaattatttgtatgaaaataattcaaacagtactatagctgaaaatgacaaaaatcatctctccgaaaaacaagatacttatttaagtaacagtagcatgtctaacagctattcctaccattctgatgaggtatataatgattcaggatatctctcaaaaaataaacttgattctggtattgagccagtattgaagaatgttgaagatcaaaaaaacactagtttttccaaagtaatatccaatgtaaaagatgcaaatgcatacccacaaactgtaaatgaagatatttgcgttgaggaacttgtgactagctcttcaccctgcaaaaataaaaatgcagccattaaattgtccatatctaatagtaataattttgaggtagggccacctgcatttaggatagccagtggtaaaatcgtttgtgtttcacatgaaacaattaaaaaagtgaaagacatatttacagacagtttcagtaaagtaattaaggaaaacaacgagaataaatcaaaaatttgccaaacgaaaattatggcaggttgttacgaggcattggatgattcagaggatattcttcataactctctagataatgatgaatgtagcacgcattcacataaggtttttgctgacattcagagtgaagaaattttacaacataaccaaaatatgtctggattggagaaagtttctaaaatatcaccttgtgatgttagtttggaaacttcagatatatgtaaatgtagtatagggaagcttcataagtcagtctcatctgcaaatacttgtgggatttttagcacagcaagtggaaaatctgtccaggtatcagatgcttcattacaaaacgcaagacaagtgttttctgaaatagaagatagtaccaagcaagtcttttccaaagtattgtttaaaagtaacgaacattcagaccagctcacaagagaagaaaatactgctatacgtactccagaacatttaatatcccaaaaaggcttttcatataatgtggtaaattcatctgctttctctggatttagtacagcaagtggaaagcaagtttccattttagaaagttccttacacaaagttaagggagtgttagaggaatttgatttaatcagaactgagcatagtcttcactattcacctacgtctagacaaaatgtatcaaaaatacttcctcgtgttgataagagaaacccagagcactgtgtaaactcagaaatggaaaaaacctgcagtaaagaatttaaattatcaaataacttaaatgttgaaggtggttcttcagaaaataatcactctattaaagtttctccatatctctctcaatttcaacaagacaaacaacagttggtattaggaaccaaagtctcacttgttgagaacattcatgttttgggaaaagaacaggcttcacctaaaaacgtaaaaatggaaattggtaaaactgaaactttttctgatgttcctgtgaaaacaaatatagaagtttgttctacttactccaaagattcagaaaactactttgaaacagaagcagtagaaattgctaaagcttttatggaagatgatgaactgacagattctaaactgccaagtcatgccacacattctctttttacatgtcccgaaaatgaggaaatggttttgtcaaattcaagaattggaaaaagaagaggagagccccttatcttagtgggagaaccctcaatcaaaagaaacttattaaatgaatttgacaggataatagaaaatcaagaaaaatccttaaaggcttcaaaaagcactccagatggcacaataaaagatcgaagattgtttatgcatcatgtttctttagagccgattacctgtgtaccctttcgcacaactaaggaacgtcaagagatacagaatccaaattttaccgcacctggtcaagaatttctgtctaaatctcatttgtatgaacatctgactttggaaaaatcttcaagcaatttagcagtttcaggacatccattttatcaagtttctgctacaagaaatgaaaaaatgagacacttgattactacaggcagaccaaccaaagtctttgttccaccttttaaaactaaatcacattttcacagagttgaacagtgtgttaggaatattaacttggaggaaaacagacaaaagcaaaacattgatggacatggctctgatgatagtaaaaataagattaatgacaatgagattcatcagtttaacaaaaacaactccaatcaagcagcagctgtaactttcacaaagtgtgaagaagaacctttagatttaattacaagtcttcagaatgccagagatatacaggatatgcgaattaagaagaaacaaaggcaacgcgtctttccacagccaggcagtctgtatcttgcaaaaacatccactctgcctcgaatctctctgaaagcagcagtaggaggccaagttccctctgcgtgttctcataaacagctgtatacgtatggcgtttctaaacattgcataaaaattaacagcaaaaatgcagagtcttttcagtttcacactgaagattattttggtaaggaaagtttatggactggaaaaggaatacagttggctgatggtggatggctcataccctccaatgatggaaaggctggaaaagaagaattttatagggctctgtgtgacactccaggtgtggatccaaagcttatttctagaatttgggtttataatcactatagatggatcatatggaaactggcagctatggaatgtgcctttcctaaggaatttgctaatagatgcctaagcccagaaagggtgcttcttcaactaaaatacagatatgatacggaaattgatagaagcagaagatcggctataaaaaagataatggaaagggatgacacagctgcaaaaacacttgttctctgtgtttctgacataatttcattgagcgcaaatatatctgaaacttctagcaataaaactagtagtgcagatacccaaaaagtggccattattgaacttacagatgggtggtatgctgttaaggcccagttagatcctcccctcttagctgtcttaaagaatggcagactgacagttggtcagaagattattcttcatggagcagaactggtgggctctcctgatgcctgtacacctcttgaagccccagaatctcttatgttaaagatttctgctaacagtactcggcctgctcgctggtataccaaacttggattctttcctgaccctagaccttttcctctgcccttatcatcgcttttcagtgatggaggaaatgttggttgtgttgatgtaattattcaaagagcataccctatacagtggatggagaagacatcatctggattatacatatttcgcaatgaaagagaggaagaaaaggaagcagcaaaatatgtggaggcccaacaaaagagactagaagccttattcactaaaattcaggaggaatttgaagaacatgaagaaaacacaacaaaaccatatttaccatcacgtgcactaacaagacagcaagttcgtgctttgcaagatggtgcagagctttatgaagcagtgaagaatgcagcagacccagcttaccttgagggttatttcagtgaagagcagttaagagccttgaataatcacaggcaaatgttgaatgataagaaacaagctcagatccagttggaaattaggaaggccatggaatctgctgaacaaaaggaacaaggtttatcaagggatgtcacaaccgtgtggaagttgcgtattgtaagctattcaaaaaaagaaaaagattcagttatactgagtatttggcgtccatcatcagatttatattctctgttaacagaaggaaagagatacagaatttatcatcttgcaacttcaaaatctaaaagtaaatctgaaagagctaacatacagttagcagcgacaaaaaaaactcagtatcaacaactaccggtttcagatgaaattttatttcagatttaccagccacgggagccccttcacttcagcaaatttttagatccagactttcagccatcttgttctgaggtggacctaataggatttgtcgtttctgttgtgaaaaaaacaggacttgcccctttcgtctatttgtcagacgaatgttacaatttactggcaataaagttttggatagaccttaatgaggacattattaagcctcatatgttaattgctgcaagcaacctccagtggcgaccagaatccaaatcaggccttcttactttatttgctggagatttttctgtgttttctgctagtccaaaagagggccactttcaagagacattcaacaaaatgaaaaatactgttgagaatattgacatactttgcaatgaagcagaaaacaagcttatgcatatactgcatgcaaatgatcccaagtggtccaccccaactaaagactgtacttcagggccgtacactgctcaaatcattcctggtacaggaaacaagcttctgatgtcttctcctaattgtgagatatattatcaaagtcctttatcactttgtatggccaaaaggaagtctgtttccacacctgtctcagcccagatgacttcaaagtcttgtaaaggggagaaagagattgatgaccaaaagaactgcaaaaagagaagagccttggatttcttgagtagactgcctttacctccacctgttagtcccatttgtacatttgtttctccggctgcacagaaggcatttcagccaccaaggagttgtggcaccaaatacgaaacacccataaagaaaaaagaactgaattctcctcagatgactccatttaaaaaattcaatgaaatttctcttttggaaagtaattcaatagctgacgaagaacttgcattgataaatacccaagctcttttgtctggttcaacaggagaaaaacaatttatatctgtcagtgaatccactaggactgctcccaccagttcagaagattatctcagactgaaacgacgttgtactacatctctgatcaaagaacaggagagttcccaggccagtacggaagaatgtgagaaaaataagcaggacacaattacaactaaaaaatatatctaagcatttgcaaaggcgacaataaattattgacgcttaacctttccagtttataagactggaatataatttcaaaccacacattagtacttatgttgcacaatgagaaaagaaattagtttcaaatttacctcagcgtttgtgtatcgggcaaaaatcgttttgcccgattccgtattggtatacttttgcttcagttgcatatcttaaaactaaatgtaatttattaactaatcaagaaaaacatctttggctgagctcggtggctcatgcctgtaatcccaacactttgagaagctgaggtgggaggagtgcttgaggccaggagttcaagaccagcctgggcaacatagggagacccccatctttacgaagaaaaaaaaaaaggggaaaagaaaatcttttaaatctttggatttgatcactacaagtattattttacaatcaacaaaatggtcatccaaactcaaacttgagaaaatatcttgctttcaaattgacacta

SEQ ID NO: 4 - protein, 3418 aa, Homo sapiens
MPIGSKERPTFFEIFKTRCNKADLGPISLNWFEELSSEAPPYNSEPAEESEHKNNNYEPNLFKTPQRKPSYNQLASTPIIFKEQGLTLPLYQSPVKELDKFKLDLGRNVPNSRHKSLRTVKTKMDQADDVSCPLLNSCLSESPVVLQCTHVTPQRDKSVVCGSLFHTPKFVKGRQTPKHISESLGAEVDPDMSWSSSLATPPTLSSTVLIVRNEEASETVFPHDTTANVKSYFSNHDESLKKNDRFIASVTDSENTNQREAASHGFGKTSGNSFKVNSCKDHIGKSMPNVLEDEVYETVVDTSEEDSFSLCFSKCRTKNLQKVRTSKTRKKIFHEANADECEKSKNQVKEKYSFVSEVEPNDTDPLDSNVAHQKPFESGSDKISKEVVPSLACEWSQLTLSGLNGAQMEKIPLLHISSCDQNISEKDLLDTENKRKKDFLTSENSLPRISSLPKSEKPLNEETVVNKRDEEQHLESHTDCILAVKQAISGTSPVASSFQGIKKSIFRIRESPKETFNASFSGHMTDPNFKKETEASESGLEIHTVCSQKEDSLCPNLIDNGSWPATTTQNSVALKNAGLISTLKKKTNKFIYAIHDETFYKGKKIPKDQKSELINCSAQFEANAFEAPLTFANADSGLLHSSVKRSCSQNDSEEPTLSLTSSFGTILRKCSRNETCSNNTVISQDLDYKEAKCNKEKLQLFITPEADSLSCLQEGQCENDPKSKKVSDIKEEVLAAACHPVQHSKVEYSDTDFQSQKSLLYDHENASTLILTPTSKDVLSNLVMISRGKESYKMSDKLKGNNYESDVELTKNIPMEKNQDVCALNENYKNVELLPPEKYMRVASPSRKVQFNQNTNLRVIQKNQEETTSISKITVNPDSEELFSDNENNFVFQVANERNNLALGNTKELHETDLTCVNEPIFKNSTMVLYGDTGDKQATQVSIKKDLVYVLAEENKNSVKQHIKMTLGQDLKSDISLNIDKIPEKNNDYMNKWAGLLGPISNHSFGGSFRTASNKEIKLSEHNIKKSKMFFKDIEEQYPTSLACVEIVNTLALDNQKKLSKPQSINTVSAHLQSSVVVSDCKNSHITPQMLFSKQDFNSNHNLTPSQKAEITELSTILEESGSQFEFTQFRKPSYILQKSTFEVPENQMTILKTTSEECRDADLHVIMNAPSIGQVDSSKQFEGTVEIKRKFAGLLKNDCNKSASGYLTDENEVGFRGFYSAHGTKLNVSTEALQKAVKLFSDIENISEETSAEVHPISLSSSKCHDSVVSMFKIENHNDKTVSEKNNKCQLILQNNIEMTTGTFVEEITENYKRNTENEDNKYTAASRNSHNLEFDGSDSSKNDTVCIHKDETDLLFTDQHNICLKLSGQFMKEGNTQIKEDLSDLTFLEVAKAQEACHGNTSNKEQLTATKTEQNIKDFETSDTFFQTASGKNISVAKESFNKIVNFFDQKPEELHNFSLNSELHSDIRKNKMDILSYEETDIVKHKILKESVPVGTGNQLVTFQGQPERDEKIKEPTLLGFHTASGKKVKIAKESLDKVKNLFDEKEQGTSEITSFSHQWAKTLKYREACKDLELACETIEITAAPKCKEMQNSLNNDKNLVSIETVVPPKLLSDNLCRQTENLKTSKSIFLKVKVHENVEKETAKSPATCYTNQSPYSVIENSALAFYTSCSRKTSVSQTSLLEAKKWLREGIFDGQPERINTADYVGNYLYENNSNSTIAENDKNHLSEKQDTYLSNSSMSNSYSYHSDEVYNDSGYLSKNKLDSGIEPVLKNVEDQKNTSFSKVISNVKDANAYPQTVNEDICVEELVTSSSPCKNKNAAIKLSISNSNNFEVGPPAFRIASGKIVCVSHETIKKVKDIFTDSFSKVIKENNENKSKICQTKIMAGCYEALDDSEDILHNSLDNDECSTHSHKVFADIQSEEILQHNQNMSGLEKVSKISPCDVSLETSDICKCSIGKLHKSVSSANTCGIFSTASGKSVQVSDASLQNARQVFSEIEDSTKQVFSKVLFKSNEHSDQLTREENTAIRTPEHLISQKGFSYNVVNSSAFSGFSTASGKQVSILESSLHKVKGVLEEFDLIRTEHSLHYSPTSRQNVSKILPRVDKRNPEHCVNSEMEKTCSKEFKLSNNLNVEGGSSENNHSIKVSPYLSQFQQDKQQLVLGTKVSLVENIHVLGKEQASPKNVKMEIGKTETFSDVPVKTNIEVCSTYSKDSENYFETEAVEIAKAFMEDDELTDSKLPSHATHSLFTCPENEEMVLSNSRIGKRRGEPLILVGEPSIKRNLLNEFDRIIENQEKSLKASKSTPDGTIKDRRLFMHHVSLEPITCVPFRTTKERQEIQNPNFTAPGQEFLSKSHLYEHLTLEKSSSNLAVSGHPFYQVSATRNEKMRHLITTGRPTKVFVPPFKTKSHFHRVEQCVRNINLEENRQKQNIDGHGSDDSKNKINDNEIHQFNKNNSNQAAAVTFTKCEEEPLDLITSLQNARDIQDMRIKKKQRQRVFPQPGSLYLAKTSTLPRISLKAAVGGQVPSACSHKQLYTYGVSKHCIKINSKNAESFQFHTEDYFGKESLWTGKGIQLADGGWLIPSNDGKAGKEEFYRALCDTPGVDPKLISRIWVYNHYRWIIWKLAAMECAFPKEFANRCLSPERVLLQLKYRYDTEIDRSRRSAIKKIMERDDTAAKTLVLCVSDIISLSANISETSSNKTSSADTQKVAIIELTDGWYAVKAQLDPPLLAVLKNGRLTVGQKIILHGAELVGSPDACTPLEAPESLMLKISANSTRPARWYTKLGFFPDPRPFPLPLSSLFSDGGNVGCVDVIIQRAYPIQWMEKTSSGLYIFRNEREEEKEAAKYVEAQQKRLEALFTKIQEEFEEHEENTTKPYLPSRALTRQQVRALQDGAELYEAVKNAADPAYLEGYFSEEQLRALNNHRQMLNDKKQAQIQLEIRKAMESAEQKEQGLSRDVTTVWKLRIVSYSKKEKDSVILSIWRPSSDLYSLLTEGKRYRIYHLATSKSKSKSERANIQLAATKKTQYQQLPVSDEILFQIYQPREPLHFSKFLDPDFQPSCSEVDLIGFVVSVVKKTGLAPFVYLSDECYNLLAIKFWIDLNEDIIKPHMLIAASNLQWRPESKSGLLTLFAGDFSVFSASPKEGHFQETFNKMKNTVENIDILCNEAENKLMHILHANDPKWSTPTKDCTSGPYTAQIIPGTGNKLLMSSPNCEIYYQSPLSLCMAKRKSVSTPVSAQMTSKSCKGEKEIDDQKNCKKRRALDFLSRLPLPPPVSPICTFVSPAAQKAFQPPRSCGTKYETPIKKKELNSPQMTPFKKFNEISLLESNSIADEELALINTQALLSGSTGEKQFISVSESTRTAPTSSEDYLRLKRRCTTSLIKEQESSQASTEECEKNKQDTITTKKYI